SEQUENCE LISTING (131 sequences)

SEQ ID NO: 1 - DNA, 40 bp, Artificial Sequence
  synthetic oligo
tgtaaaacgacggccagttgaggccgatattaatccggtg

SEQ ID NO: 2 - DNA, 43 bp, Artificial Sequence
  synthetic oligo
caggaaacagctatgacctcgctactatggcctgtgtttctca

SEQ ID NO: 3 - DNA, 41 bp, Artificial Sequence
  synthetic oligo
tgtaaaacgacggccagtccagataggcagaaatgggcttg

SEQ ID NO: 4 - DNA, 42 bp, Artificial Sequence
  synthetic oligo
caggaaacagctatgaccggttccaagtcattcccagtagca

SEQ ID NO: 5 - DNA, 38 bp, Artificial Sequence
  synthetic oligo
tgtaaaacgacggccagtgaaatgggcttgaatagtta

SEQ ID NO: 6 - DNA, 39 bp, Artificial Sequence
  synthetic oligo
caggaaacagctatgacccttccctagtgtggtaacctc

SEQ ID NO: 7 - DNA, 42 bp, Artificial Sequence
  synthetic oligo
tgtaaaacgacggccagtatgagccactgtacccagcctaat

SEQ ID NO: 8 - DNA, 44 bp, Artificial Sequence
  synthetic oligo
caggaaacagctatgacctttgggtttaaacaagcgttaagaaa

SEQ ID NO: 9 - DNA, 38 bp, Artificial Sequence
  synthetic oligo
tgtaaaacgacggccagtgtgctgagattgcaggcatg

SEQ ID NO: 10 - DNA, 38 bp, Artificial Sequence
  synthetic oligo
caggaaacagctatgaccatgaatatggatcacatctc

SEQ ID NO: 11 - DNA, 39 bp, Artificial Sequence
  synthetic oligo
tgtaaaacgacggccagtcaacaccagcccgtttatggc

SEQ ID NO: 12 - DNA, 44 bp, Artificial Sequence
  synthetic oligo
caggaaacagctatgacctgcagaagaggataggcagaaactca

SEQ ID NO: 13 - DNA, 39 bp, Artificial Sequence
  synthetic oligo
tgtaaaacgacggccagtccggctctcggttataagatg

SEQ ID NO: 14 - DNA, 38 bp, Artificial Sequence
  synthetic oligo
caggaaacagctatgaccaaataaacaccagccagccg

SEQ ID NO: 15 - DNA, 39 bp, Artificial Sequence
  synthetic oligo
tgtaaaacgacggccagtggaaggctgctcaccaaacca

SEQ ID NO: 16 - DNA, 42 bp, Artificial Sequence
  synthetic oligo
caggaaacagctatgaccggcaaagctaattctctcttccca

SEQ ID NO: 17 - DNA, 41 bp, Artificial Sequence
  synthetic oligo
tgtaaaacgacggccagttgtcaggacaaagtccggattga

SEQ ID NO: 18 - DNA, 43 bp, Artificial Sequence
  synthetic oligo
caggaaacagctatgaccgacatgactgtggttcaagtttggc

SEQ ID NO: 19 - DNA, 42 bp, Artificial Sequence
  synthetic oligo
tgtaaaacgacggccagttgatggatatactgcgttggtggg

SEQ ID NO: 20 - DNA, 45 bp, Artificial Sequence
  synthetic oligo
caggaaacagctatgacctggcctacagtatttcttcaggctaac

SEQ ID NO: 21 - DNA, 38 bp, Artificial Sequence
  synthetic oligo
tgtaaaacgacggccagtgttgtatctgacctagtaac

SEQ ID NO: 22 - DNA, 38 bp, Artificial Sequence
  synthetic oligo
caggaaacagctatgacccctacagtatttcttcaggc

SEQ ID NO: 23 - DNA, 38 bp, Artificial Sequence
  synthetic oligo
tgtaaaacgacggccagttaataaccaagaaaggcttg

SEQ ID NO: 24 - DNA, 38 bp, Artificial Sequence
  synthetic oligo
caggaaacagctatgaccattcattcattaaaatctaa

SEQ ID NO: 25 - DNA, 40 bp, Artificial Sequence
  synthetic oligo
tgtaaaacgacggccagtgcttgaaatcagttgccagcct

SEQ ID NO: 26 - DNA, 44 bp, Artificial Sequence
  synthetic oligo
caggaaacagctatgacctgggagagaaatactgtccattccac

SEQ ID NO: 27 - DNA, 45 bp, Artificial Sequence
  synthetic oligo
tgtaaaacgacggccagtaaatttctctttgctgtatgccagttt

SEQ ID NO: 28 - DNA, 40 bp, Artificial Sequence
  synthetic oligo
caggaaacagctatgacccccaagagcagaagtcaaacca

SEQ ID NO: 29 - DNA, 39 bp, Artificial Sequence
  synthetic oligo
tgtaaaacgacggccagtcctggagggcctacttgagca

SEQ ID NO: 30 - DNA, 44 bp, Artificial Sequence
  synthetic oligo
caggaaacagctatgaccaatgtcatcagagagaaaccagaagc

SEQ ID NO: 31 - DNA, 40 bp, Artificial Sequence
  synthetic oligo
tgtaaaacgacggccagttggcctgaaatggacatcaaca

SEQ ID NO: 32 - DNA, 44 bp, Artificial Sequence
  synthetic oligo
caggaaacagctatgacctgtgtctgttacttgaaagaggcaag

SEQ ID NO: 33 - DNA, 37 bp, Artificial Sequence
  synthetic oligo
tgtaaaacgacggccagttttggcccatcctttccaa

SEQ ID NO: 34 - DNA, 44 bp, Artificial Sequence
  synthetic oligo
caggaaacagctatgacctgaaatatctgaattctggaccagcc

SEQ ID NO: 35 - DNA, 43 bp, Artificial Sequence
  synthetic oligo
tgtaaaacgacggccagttctcttcctgtatccctctcaggca

SEQ ID NO: 36 - DNA, 38 bp, Artificial Sequence
  synthetic oligo
caggaaacagctatgaccggagtcccgactgctgtgaa

SEQ ID NO: 37 - DNA, 40 bp, Artificial Sequence
  synthetic oligo
tgtaaaacgacggccagtccatggaacaaacaaggttggc

SEQ ID NO: 38 - DNA, 40 bp, Artificial Sequence
  synthetic oligo
caggaaacagctatgaccgctaccactgggaaccaggagc

SEQ ID NO: 39 - DNA, 43 bp, Artificial Sequence
  synthetic oligo
tgtaaaacgacggccagtaagcaacctctagttgcctgtcaga

SEQ ID NO: 40 - DNA, 44 bp, Artificial Sequence
  synthetic oligo
caggaaacagctatgacctctgaaggctgcactagacagagaca

SEQ ID NO: 41 - DNA, 42 bp, Artificial Sequence
  synthetic oligo
tgtaaaacgacggccagtggcttgactggagtgaaaggtttg

SEQ ID NO: 42 - DNA, 41 bp, Artificial Sequence
  synthetic oligo
caggaaacagctatgacccaggctgtggtatcctgctctcc

SEQ ID NO: 43 - DNA, 38 bp, Artificial Sequence
  synthetic oligo
tgtaaaacgacggccagtcataatgcttgctctgatag

SEQ ID NO: 44 - DNA, 38 bp, Artificial Sequence
  synthetic oligo
caggaaacagctatgacctaactcagcagcatctcagg

SEQ ID NO: 45 - DNA, 41 bp, Artificial Sequence
  synthetic oligo
tgtaaaacgacggccagtcaggaatgggaagtgggaactga

SEQ ID NO: 46 - DNA, 41 bp, Artificial Sequence
  synthetic oligo
caggaaacagctatgacctccttcacgcttacccaggagtt

SEQ ID NO: 47 - DNA, 39 bp, Artificial Sequence
  synthetic oligo
tgtaaaacgacggccagttttgtgggtttcccaccatct

SEQ ID NO: 48 - DNA, 39 bp, Artificial Sequence
  synthetic oligo
caggaaacagctatgacctgctcaaccctcatgaagcca

SEQ ID NO: 49 - DNA, 43 bp, Artificial Sequence
  synthetic oligo
tgtaaaacgacggccagtggtgctttcttgtaaagtgtgatgg

SEQ ID NO: 50 - DNA, 42 bp, Artificial Sequence
  synthetic oligo
caggaaacagctatgacccaaacccggaacagaaagtaaagc

SEQ ID NO: 51 - DNA, 38 bp, Artificial Sequence
  synthetic oligo
tgtaaaacgacggccagtgtgtgatgggactcttaaag

SEQ ID NO: 52 - DNA, 38 bp, Artificial Sequence
  synthetic oligo
caggaaacagctatgaccgttgctactctcctgaactc

SEQ ID NO: 53 - DNA, 38 bp, Artificial Sequence
  synthetic oligo
tgtaaaacgacggccagtgtagattctcgcctctattg

SEQ ID NO: 54 - DNA, 38 bp, Artificial Sequence; identical to SEQ ID NO: 52
  synthetic oligo

SEQ ID NO: 55 - DNA, 38 bp, Artificial Sequence
  synthetic oligo
tgtaaaacgacggccagtagcgggaggaagcgagaggt

SEQ ID NO: 56 - DNA, 40 bp, Artificial Sequence
  synthetic oligo
caggaaacagctatgacccccagacgtacgtgacaaacca

SEQ ID NO: 57 - DNA, 38 bp, Artificial Sequence
  synthetic oligo
tgtaaaacgacggccagtcgggtggctggagtgaagtg

SEQ ID NO: 58 - DNA, 42 bp, Artificial Sequence
  synthetic oligo
caggaaacagctatgacccagtcttccttctaccctggtccc

SEQ ID NO: 59 - DNA, 38 bp, Artificial Sequence
  synthetic oligo
tgtaaaacgacggccagtccgaggcacagctggaacag

SEQ ID NO: 60 - DNA, 38 bp, Artificial Sequence
  synthetic oligo
caggaaacagctatgaccggaggcctgccatcttgtga

SEQ ID NO: 61 - DNA, 42 bp, Artificial Sequence
  synthetic oligo
tgtaaaacgacggccagtcataggacaaggtatgggagtggg

SEQ ID NO: 62 - DNA, 45 bp, Artificial Sequence
  synthetic oligo
caggaaacagctatgacctccagacagggaagagtctagtgagaa

SEQ ID NO: 63 - DNA, 41 bp, Artificial Sequence
  synthetic oligo
tgtaaaacgacggccagtggaggaaggcaaatttgtgatga

SEQ ID NO: 64 - DNA, 38 bp, Artificial Sequence
  synthetic oligo
caggaaacagctatgaccagctggccagtcctggcttt

SEQ ID NO: 65 - DNA, 38 bp, Artificial Sequence
  synthetic oligo
tgtaaaacgacggccagtccaagggctgcctctgatgg

SEQ ID NO: 66 - DNA, 39 bp, Artificial Sequence
  synthetic oligo
caggaaacagctatgaccgccacactctggagctggaca

SEQ ID NO: 67 - DNA, 38 bp, Artificial Sequence
  synthetic oligo
tgtaaaacgacggccagtcagccagggcacagctgatt

SEQ ID NO: 68 - DNA, 38 bp, Artificial Sequence
  synthetic oligo
caggaaacagctatgacctagcctggcatccagggaca

SEQ ID NO: 69 - DNA, 44 bp, Artificial Sequence
  synthetic oligo
tgtaaaacgacggccagttgtggctgtttaatgtttattgtcca

SEQ ID NO: 70 - DNA, 41 bp, Artificial Sequence
  synthetic oligo
caggaaacagctatgacctgaacctgtgacagcgatgtgtt

SEQ ID NO: 71 - DNA, 38 bp, Artificial Sequence
  synthetic oligo
tgtaaaacgacggccagttacgttcccatgccgcactc

SEQ ID NO: 72 - DNA, 39 bp, Artificial Sequence
  synthetic oligo
caggaaacagctatgacctcagacgggagggtaaagggc

SEQ ID NO: 73 - DNA, 38 bp, Artificial Sequence
  synthetic oligo
tgtaaaacgacggccagtggatgaatcaagcccaggca

SEQ ID NO: 74 - DNA, 38 bp, Artificial Sequence
  synthetic oligo
caggaaacagctatgacctgccaccaaacgagacagca

SEQ ID NO: 75 - DNA, 40 bp, Artificial Sequence
  synthetic oligo
tgtaaaacgacggccagtcaccacgtcctctcgtttcctt

SEQ ID NO: 76 - DNA, 40 bp, Artificial Sequence
  synthetic oligo
caggaaacagctatgaccaggatctcacaaggctccctcc

SEQ ID NO: 77 - DNA, 38 bp, Artificial Sequence
  synthetic oligo
tgtaaaacgacggccagtctatgggccccggctagagg

SEQ ID NO: 78 - DNA, 39 bp, Artificial Sequence
  synthetic oligo
caggaaacagctatgaccgcctcgtgcactcctcgcgaa

SEQ ID NO: 79 - DNA, 38 bp, Artificial Sequence
  synthetic oligo
tgtaaaacgacggccagtcccagaagctgggccctttc

SEQ ID NO: 80 - DNA, 38 bp, Artificial Sequence
  synthetic oligo
caggaaacagctatgaccccgccctggattctggtcat

SEQ ID NO: 81 - DNA, 39 bp, Artificial Sequence
  synthetic oligo
tgtaaaacgacggccagttggcatcgactgccttgagaa

SEQ ID NO: 82 - DNA, 38 bp, Artificial Sequence
  synthetic oligo
caggaaacagctatgacctgaccactgttgggaacgcc

SEQ ID NO: 83 - DNA, 38 bp, Artificial Sequence
  synthetic oligo
tgtaaaacgacggccagtaggtcctggcgtcccacatc

SEQ ID NO: 84 - DNA, 37 bp, Artificial Sequence
  synthetic oligo
caggaaacagctatgacccgtgggtggagttgggctc

SEQ ID NO: 85 - DNA, 37 bp, Artificial Sequence
  synthetic oligo
tgtaaaacgacggccagtacgcatccgtggcttcccg

SEQ ID NO: 86 - DNA, 38 bp, Artificial Sequence
  synthetic oligo
caggaaacagctatgaccggcagatggtgacctcaggc

SEQ ID NO: 87 - DNA, 38 bp, Artificial Sequence
  synthetic oligo
tgtaaaacgacggccagtgttgggaaagcctcgtggga

SEQ ID NO: 88 - DNA, 38 bp, Artificial Sequence
  synthetic oligo
caggaaacagctatgaccgcgagctcctcacagcctga

SEQ ID NO: 89 - DNA, 38 bp, Artificial Sequence
  synthetic oligo
tgtaaaacgacggccagtcagctctgactgctcagctc

SEQ ID NO: 90 - DNA, 38 bp, Artificial Sequence
  synthetic oligo
caggaaacagctatgaccgccatggagtcgatgagctg

SEQ ID NO: 91 - DNA, 39 bp, Artificial Sequence
  synthetic oligo
tgtaaaacgacggccagtcagatcatgcaccgaggtaag

SEQ ID NO: 92 - DNA, 39 bp, Artificial Sequence
  synthetic oligo
caggaaacagctatgaccgacagctgcgcaggagacatg

SEQ ID NO: 93 - DNA, 38 bp, Artificial Sequence
  synthetic oligo
tgtaaaacgacggccagtgaccacagtcggcaccatcc

SEQ ID NO: 94 - DNA, 38 bp, Artificial Sequence
  synthetic oligo
caggaaacagctatgaccctgcggtcaacaccagctcc

SEQ ID NO: 95 - DNA, 38 bp, Artificial Sequence
  synthetic oligo
tgtaaaacgacggccagtgggaaggagctgggcttgtg

SEQ ID NO: 96 - DNA, 38 bp, Artificial Sequence
  synthetic oligo
caggaaacagctatgacccgcctccaatttaggctggc

SEQ ID NO: 97 - DNA, 38 bp, Artificial Sequence
  synthetic oligo
tgtaaaacgacggccagtcagtgcctctgcacacccgc

SEQ ID NO: 98 - DNA, 38 bp, Artificial Sequence
  synthetic oligo
caggaaacagctatgaccctcttgctctggtcagagag

SEQ ID NO: 99 - DNA, 38 bp, Artificial Sequence
  synthetic oligo
tgtaaaacgacggccagtacagggcctgcaatcctgct

SEQ ID NO: 100 - DNA, 38 bp, Artificial Sequence
  synthetic oligo
caggaaacagctatgaccagttggcatgtggtgtcggc

SEQ ID NO: 101 - DNA, 38 bp, Artificial Sequence
  synthetic oligo
tgtaaaacgacggccagtccagagctgttcccacgcac

SEQ ID NO: 102 - DNA, 38 bp, Artificial Sequence
  synthetic oligo
caggaaacagctatgaccccctggcaccaaagtgcaga

SEQ ID NO: 103 - DNA, 38 bp, Artificial Sequence
  synthetic oligo
tgtaaaacgacggccagtgctcagggatgtcctctccg

SEQ ID NO: 104 - DNA, 41 bp, Artificial Sequence
  synthetic oligo
caggaaacagctatgaccggaagggagggtctcctctgttt

SEQ ID NO: 105 - DNA, 37 bp, Artificial Sequence
  synthetic oligo
tgtaaaacgacggccagtgtcctactgtgactccagg

SEQ ID NO: 106 - DNA, 37 bp, Artificial Sequence
  synthetic oligo
caggaaacagctatgaccgccagcctgtcctcagctg

SEQ ID NO: 107 - DNA, 21 bp, Artificial Sequence
  synthetic oligo
atgactgagtacaaactggtg

SEQ ID NO: 108 - DNA, 23 bp, Artificial Sequence
  synthetic oligo
ttacatcaccacacatggcaatc

SEQ ID NO: 109 - DNA, 59 bp, Artificial Sequence
  synthetic oligo
caccatggattacaaggatgacgacgataagggaatgactgagtacaaactggtggtgg

SEQ ID NO: 110 - DNA, 23 bp, Artificial Sequence
  synthetic oligo
ttacatcaccacacatggcaatc

SEQ ID NO: 111 - DNA, 60 bp, Artificial Sequence
  synthetic oligo
gtttgttggacatactggatacagctggaaaagaagagtacagtgccatgagagaccaat

SEQ ID NO: 112 - DNA, 59 bp, Artificial Sequence
  synthetic oligo
ttggtctctcatggcactgtactcttcttttccagctgtatccagtatgtccaacaaac

SEQ ID NO: 113 - DNA, 60 bp, Artificial Sequence
  synthetic oligo
gtttgttggacatactggatacagctggacgagaagagtacagtgccatgagagaccaat

SEQ ID NO: 114 - DNA, 60 bp, Artificial Sequence
  synthetic oligo
attggtctctcatggcactgtactcttctcgtccagctgtatccagtatgtccaacaaac

SEQ ID NO: 115 - DNA, 60 bp, Artificial Sequence
  synthetic oligo
gttacgggattccattcattgaaacctcaaccaagaccagacagggtgttgaagatgctt

SEQ ID NO: 116 - DNA, 60 bp, Artificial Sequence
  synthetic oligo
aagcatcttcaacaccctgtctggtcttggttgaggtttcaatgaatggaatcccgtaac

SEQ ID NO: 117 - DNA, 59 bp, Artificial Sequence; identical to SEQ ID NO: 109
  synthetic oligo

SEQ ID NO: 118 - DNA, 38 bp, Artificial Sequence
  synthetic oligo
gaattcggtaccatgcccaagaagaagccgacgcccat

SEQ ID NO: 119 - DNA, 41 bp, Artificial Sequence
  synthetic oligo
gttaacggatccttagacgccagcagcatgggttggtgtgc

SEQ ID NO: 120 - DNA, 45 bp, Artificial Sequence
  synthetic oligo
cgccttgaggcctttcttaccccgaagcagaaggtgggagaactg

SEQ ID NO: 121 - DNA, 45 bp, Artificial Sequence
  synthetic oligo
cagttctcccaccttctgcttcggggtaagaaaggcctcaaggcg

SEQ ID NO: 122 - DNA, 48 bp, Artificial Sequence
  synthetic oligo
gaggcctttcttacccagaagcaggtgggagaactgaaggatgacgac

SEQ ID NO: 123 - DNA, 48 bp, Artificial Sequence
  synthetic oligo
gtcgtcatccttcagttctcccacctgcttctgggtaagaaaggcctc

SEQ ID NO: 124 - DNA, 45 bp, Artificial Sequence
  synthetic oligo
ggccttaaccagcccagcacatcaacccatgctgctggcgtctaa

SEQ ID NO: 125 - DNA, 45 bp, Artificial Sequence
  synthetic oligo
ttagacgccagcagcatgggttgatgtgctgggctggttaaggcc

SEQ ID NO: 126 - DNA, 51 bp, Artificial Sequence
  synthetic oligo
agggagctgcaggttctgcatgagtccaactctccgtacatcgtgggcttc

SEQ ID NO: 127 - DNA, 51 bp, Artificial Sequence
  synthetic oligo
gaagcccacgatgtacggagagttggactcatgcagaacctgcagctccct

SEQ ID NO: 128 - DNA, 570 bp, Homo sapiens
atgactgagtacaaactggtggtggttggagcaggtggtgttgggaaaagcgcactgacaatccagctaatccagaaccactttgtagatgaatatgatcccaccatagaggattcttacagaaaacaagtggttatagatggtgaaacctgtttgttggacatactggatacagctggacaagaagagtacagtgccatgagagaccaatacatgaggacaggcgaaggcttcctctgtgtatttgccatcaataatagcaagtcatttgcggatattaacctctacagggagcagattaagcgagtaaaagactcggatgatgtacctatggtgctagtgggaaacaagtgtgatttgccaacaaggacagttgatacaaaacaagcccacgaactggccaagagttacgggattccattcattgaaacctcagccaagaccagacagggtgttgaagatgctttttacacactggtaagagaaatacgccagtaccgaatgaaaaaactcaacagcagtgatgatgggactcagggttgtatgggattgccatgtgtggtgatgtaa

SEQ ID NO: 129 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 130 - DNA, 1182 bp, Homo sapiens
atgcccaagaagaagccgacgcccatccagctgaacccggcccccgacggctctgcagttaacgggaccagctctgcggagaccaacttggaggccttgcagaagaagctggaggagctagagcttgatgagcagcagcgaaagcgccttgaggcctttcttacccagaagcagaaggtgggagaactgaaggatgacgactttgagaagatcagtgagctgggggctggcaatggcggtgtggtgttcaaggtctcccacaagccttctggcctggtcatggccagaaagctaattcatctggagatcaaacccgcaatccggaaccagatcataagggagctgcaggttctgcatgagtgcaactctccgtacatcgtgggcttctatggtgcgttctacagcgatggcgagatcagtatctgcatggagcacatggatggaggttctctggatcaagtcctgaagaaagctggaagaattcctgaacaaattttaggaaaagttagcattgctgtaataaaaggcctgacatatctgagggagaagcacaagatcatgcacagagatgtcaagccctccaacatcctagtcaactcccgtggggagatcaagctctgtgactttggggtcagcgggcagctcatcgactccatggccaactccttcgtgggcacaaggtcctacatgtcgccagaaagactccaggggactcattactctgtgcagtcagacatctggagcatgggactgtctctggtagagatggcggttgggaggtatcccatccctcctccagatgccaaggagctggagctgatgtttgggtgccaggtggaaggagatgcggctgagaccccacccaggccaaggacccccgggaggccccttagctcatacggaatggacagccgacctcccatggcaatttttgagttgttggattacatagtcaacgagcctcctccaaaactgcccagtggagtgttcagtctggaatttcaagattttgtgaataaatgcttaataaaaaaccccgcagagagagcagatttgaagcaactcatggttcatgcttttatcaagagatctgatgctgaggaagtggattttgcaggttggctctgctccaccatcggccttaaccagcccagcacaccaacccatgctgctggcgtctaa

SEQ ID NO: 131 - protein, 393 aa, Homo sapiens [sequence omitted]

(19) United States
(12) Patent Application Publication
(10) Pub. No.: US 2013/0231347 A1
(43) Pub. Date: Sep. 5, 2013

(54) METHOD OF TREATMENT WITH BRAF INHIBITOR

(71) Applicants: Maureen Bleam, Collegeville, PA (US); Tona M. Gilmer, Durham, NC (US); James G. Greger, Collegeville, PA (US); Sylvie G. Laquerre, King of Prussia, PA (US); Li Liu, Collegeville, PA (US)

(72) Inventors: Maureen Bleam, Collegeville, PA (US); Tona M. Gilmer, Durham, NC (US); James G. Greger, Collegeville, PA (US); Sylvie G. Laquerre, King of Prussia, PA (US); Li Liu, Collegeville, PA (US)

(73) Assignee: GLAXOSMITHKLINE INTELLECTUAL PROPERTY (NO.2) LIMITED, Middlesex (GB)

(21) Appl. No.: 13/988,116

(22) PCT Filed: Nov. 18, 2011
(86) PCT No.: PCT/US11/61408

§ 371 Date: May 17, 2013

Related U.S. Application Data
(60) Provisional application No. 61/415,620, filed on Nov. 19, 2010.
(60) Provisional application No. 61/559,072, filed on Nov. 12, 2011.

(51) Int. Cl. A61K 31/519; A61K 45/06; A61K 31/437; A61K 31/501; A61K 31/506

(52) U.S. Cl. CPC A61K 31/519; A61K 31/501; A61K 31/506; A61K 31/437; A61K 45/06
USPC 514/252.04; 514/264.1; 514/275; 514/300

(57) ABSTRACT

The disclosure is directed to a method of treating humans suffering from V600 mutant melanoma that incorporates the detection of the presence or absence of at least one mutation in at least one NRAS protein and/or at least one MEK1 protein, or a gene encoding said protein, and subsequently initiating treatment, modifying treatment, or discontinuing treatment with a chemo-therapeutic agent selected from a BRaf inhibitor or other suitable agent based upon the presence or absence of said mutation.

### FIELD OF THE INVENTION

The present invention relates to methods of treatment using BRAF inhibitors. Specifically, the invention relates to a method of testing a human subject having a susceptible carcinoma and treating the subject with a BRAF inhibitor according to the presence or absence of particular genetic variations.

### BACKGROUND OF THE INVENTION

In the era of personalized medicine, cancer treatments are increasingly focused based on genetic and other biomarkers found in the patients and tumor to be treated. Today, many treatments focus on particular cellular mechanisms rather than the more general treatments of the past.

Recently, B-Raf inhibitors have been investigated for use in treating cancer due to increased understanding of the Ras-Raf-MEK-ERK kinase pathway (known as the MAPK pathway). Particularly, the understanding that activation of Ras proteins in response to growth factors, hormones, cytokines, etc. stimulates phosphorylation and activation of Raf kinases which, in turn, phosphorylate and activate the MEK1 and MEK2 kinases which then phosphorylate and activate the ERK1 and 2 kinases.

Mutations in the MAPK substituent kinases are believed to negatively affect the growth signal functionality of the pathway, resulting in the establishment, development, and progression of a wide range of human cancers. Naturally occurring mutations in the B-Raf kinase have been observed in significant percentages of human melanomas (Davies, H., et al., Nature (2002) 9:1-6; Garnett, M. J. & Marais, R., Cancer Cell (2004) 6:313-319) and thyroid cancers (Cohen et al J. Nat. Cancer Inst. (2003) 95(8) 625-627 and Kimura et al Cancer Res. (2003) 63(7) 1454-1457), as well as at lower, but still significant, frequencies a number of other cancers.

GlaxoSmithKline has discovered a family of compounds (previously described in international patent application PCT/US2009/042682 published as international publication number WO2009/137391), including the compound of formula (I):

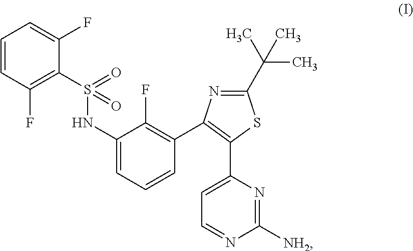 [SMILES: CC(C)(C)c1nc(-c2cccc(NS(=O)(=O)c3c(F)cccc3F)c2F)c(-c2ccnc(N)n2)s1]

referred hereinafter as Compound A.
These compounds have been found to be potent and selective RAF kinase inhibitors. In particular, the compound of formula (I) has been shown to be a potent and selective inhibitor against human wild type BRAF and CRAF as well as against several mutant forms prevalent in human melanoma and other cancers, including BRAFV600E, BRAFV600K, BRAFV600D, BRAFV600L, and BRAFV600R. The effectiveness of the compound against BRAFV600E mutations is significant in treatment of cancers such as melanoma, since the V600E mutation is the predominant mutation of BRAF, and BRAF mutations occur in the majority of melanomas.

Going forward, there remains opportunity to refine the personalized medicine approach even further through discovery and association of additional biomarkers. With particular regard to treatments with the compound of formula (I) and BRaf inhibitors of the same class, the identification of additional biomarkers and their relationship to disease and treatment could aid in focusing treatment specifically on those who stand to benefit while avoiding inefficacious treatment.

### SUMMARY OF THE INVENTION

The present invention is directed to a method of treating humans suffering from V600 mutant melanoma that incorporates the detection of the presence or absence of at least one mutation in at least one NRAS gene and/or at least one MEK1 gene, or an alteration in said protein, and subsequently initiating treatment, modifying treatment, or discontinuing treatment with a chemotherapeutic agent selected from a BRaf inhibitor or other suitable agent based upon the presence or absence of said mutation. The present invention is also directed to a method of treating humans suffering from V600 mutant melanoma that incorporates the detection of the presence or absence of at least one mutation in at least one NRAS gene and/or at least one MEK1 gene, or an alteration in said protein, and subsequently initiating treatment, modifying treatment, or discontinuing treatment with a chemotherapeutic agent selected from a BRaf inhibitor or a MEK1 inhibitor other suitable agent based upon the presence or absence of said mutation. More particularly, the invention is a method of treating V600 mutant melanoma that comprises the detection of one or a combination of NRAS mutations at codon 61 and/or 146, for instance Q61K, Q61H, Q61R, Q61L, Q61N, Q61E, Q61P, A146T, A146P, or A146V, a MEK1 deletion at codon 59, a MEK1 mutation at codon 387, for instance P387S, or MEK1 mutations at codon 56, for instance Q56P, or MEK 1 mutations at codon 121, such as C121S, or MEK 1 mutations at codon 124, such as P124L, or MEK1 mutations at codon 129, such as F129L, wherein such mutation indicates an increased likelihood of resistance to treatment with a BRaf inhibitor or a MEK1 inhibitor, and subsequent modification of said cancer treatment therapy based on said detection.

According to one embodiment of the invention, there is a method of treating a human suffering from V600 mutant melanoma comprising the steps of detecting the presence (or absence) of one or a combination of NRAS mutation at codon 61 and/or 146, for instance Q61K, Q61H, Q61R, Q61L, Q61N, Q61E, Q61P, A146T, A146P, or A146V, a MEK1 deletion at codon 59, a MEK1 mutation at codon 387, for instance P387S, or MEK1 mutations at codon 56, for instance Q56P, or MEK 1 mutations at codon 121, such as C121S, or MEK 1 mutations at codon 124, such as P124L, or MEK1 mutations at codon 129, such as F129L, and based thereon, predicting the response or duration of response (or progression-free survival) to a BRaf inhibitor or a MEK1 inhibitor.

According to another embodiment of the invention, there is a method of treating a human suffering from V600 mutant melanoma comprising the steps of detecting the magnitude of one or a combination of NRAS mutation at codon 61 and/or 146, for instance Q61K, Q61H, Q61R, Q61L, Q61N, Q61E, Q61P, A146T, A146P, or A146V, a MEK1 deletion at codon 59, a MEK1 mutation at codon 387, for instance P387S, or MEK1 mutations at codon 56, for instance Q56P, or MEK 1 mutations at codon 121, such as C121S, or MEK 1 mutations at codon 124, such as P124L, or MEK1 mutations at codon 129, such as F129L and, based thereon, predicting the response or duration of response (or progression-free survival) to a BRaf inhibitor or a MEK1 inhibitor.

According to another embodiment of the invention, there is a method of treating a human suffering from V600 mutant melanoma comprising the steps of detecting the presence (or absence) of one or a combination of NRAS mutation at codon 61 and/or 146, for instance Q61K, Q61H, Q61R, Q61L, Q61N, Q61E, Q61P, A146T, A146P, or A146V, a MEK1 deletion at codon 59, a MEK1 mutation at codon 387, for instance P387S, or MEK1 mutations at codon 56, for instance Q56P, or MEK 1 mutations at codon 121, such as C121S, or MEK 1 mutations at codon 124, such as P124L, or MEK1 mutations at codon 129, such as F129L and, based thereon, predicting the response or duration of response (or progression-free survival) to Compound A or Compound B and/or salts thereof.

According to another embodiment of the invention, there is a method of treating a human suffering from V600 mutant melanoma comprising the steps of detecting the magnitude of one or a combination of NRAS mutation at codon 61 and/or 146, for instance Q61K, Q61H, Q61R, Q61L, Q61N, Q61E, Q61P, A146T, A146P, or A146V, a MEK1 deletion at codon 59, a MEK1 mutation at codon 387, for instance P387S, or MEK1 mutations at codon 56, for instance Q56P, or MEK 1 mutations at codon 121, such as C121S, or MEK 1 mutations at codon 124, such as P124L, or MEK1 mutations at codon 129, such as F129L and, based thereon, predicting the response or duration of response (or progression-free survival) to Compound A or salts thereof.

According to another embodiment of the invention, there is a method of determining a predisposition to BRaf inhibitor or MEK1 resistance in an individual, comprising providing a sample from the individual, wherein the sample comprises DNA or RNA; and identifying the predisposition by utilizing a primer that detects a sequence indicative of said BRaf inhibitor resistance. The identifying step may be further defined as subjecting the primer to suitable polymerization conditions, such that when polymerization from the primer occurs, the sequence indicative of BRaf inhibitor or MEK1 inhibitor resistance is present in the NRAS DNA or RNA or MEK1 DNA or RNA. In specific embodiments, the individual has melanoma. This method may occur prior to BRaf inhibitor therapy. In specific embodiments, when the sequence is identified in the individual, it provides prognosis and/or treatment information.

According to another embodiment of the invention, upon identification of the resistance-conferring NRAS mutation or MEK1 mutation after initiation of treatment with a BRaf inhibitor or MEK1 inhibitor, the therapy is adjusted to circumvent at least some therapy resistance issues. For example, an alternative anticancer therapy is employed, such as an alternative chemotherapeutic, and/or a change in dosage of Compound A or salts thereof is employed.

According to an embodiment of the invention, there is a method of treating a human suffering from V600 mutant melanoma comprising the steps of administering a first BRaf inhibitor, determining if the melanoma tumor tissue of said human has at least one of an NRAS Q61 mutation, NRAS A146 mutation, MEK1 K59 deletion, MEK1 P387 mutation, or MEK1 Q56P mutation, or C121S mutation, P124L mutation, or F129L mutation and administering a combination of second BRaf inhibitor and MEK inhibitor if said mutation or deletion mutation is present. The first and second BRaf inhibitors may but are not required to be the same.

According to an embodiment of the invention, there is a method of treating a human suffering from V600 mutant melanoma comprising the steps of administering BRaf inhibitor Compound A or pharmaceutically acceptable salt thereof, determining if the melanoma tumor tissue of said human has at least one of an NRAS Q61 mutation, NRAS A146 mutation, MEK1 K59 deletion, MEK1 P387 mutation, or MEK1 Q56P mutation, or C121S mutation, P124L mutation, or F129L mutation, and administering a combination of a second BRaf inhibitor and MEK inhibitor if said mutation or deletion is present. Advantageously, the second BRaf inhibitor is BRaf inhibitor Compound A or pharmaceutically acceptable salt thereof. Advantageously, the MEK inhibitor is Compound B or a pharmaceutically acceptable salt thereof.

According to an embodiment of the invention, there is a method of treating a human suffering from V600 mutant melanoma comprising the steps of administering BRaf inhibitor Compound A or pharmaceutically acceptable salt thereof, determining if the melanoma tumor tissue of said human has at least one of an NRAS Q61 mutation, NRAS A146 mutation, MEK1 K59 deletion, MEK1 P387 mutation, or MEK1 Q56P mutation, or C121S mutation, P124L mutation, or F129L mutation, and administering a combination of BRaf inhibitor Compound A or pharmaceutically acceptable salt thereof and MEK inhibitor of Compound B or a pharmaceutically acceptable salt thereof.

According to an embodiment of the invention, there is a method of treating a human suffering from V600 mutant melanoma comprising the steps of administering N-{3-[5-(2-Amino-4-pyrimidinyl)-2-(1,1-dimethylethyl)-1,3-thiazol-4-yl]-2-fluorophenyl}-2,6-difluorobenzenesulfonamide methanesulfonate, determining if the melanoma tumor tissue of said human has at least one of an NRAS Q61 mutation, NRAS A146 mutation, MEK1 K59 deletion, MEK1 P387 mutation, or MEK1 Q56P mutation, or C121S mutation, P124L mutation, or F129L mutation, and administering a combination of N-{3-[5-(2-Amino-4-pyrimidinyl)-2-(1,1-dimethylethyl)-1,3-thiazol-4-yl]-2-fluorophenyl}-2,6-difluorobenzenesulfonamide methanesulfonate and N-{3-[3-cyclopropyl-5-(2-fluoro-4-iodo-phenylamino)-6,8-dimethyl-2,4,7-trioxo-3,4,6,7-tetrahydro-2H-pyrido[4,3-d]pyrimidin-1-yl]phenyl}acetamide as a dimethyl sulfoxide solvate.

According to an embodiment of the invention, there is a method of treating a human suffering from V600 mutant melanoma comprising the steps of administering a first BRaf inhibitor, determining if the melanoma tumor tissue of said human has at least one of an NRAS Q61 mutation, NRAS A146 mutation, MEK1 K59 deletion, or MEK1 P387 mutation, or MEK1 Q56P mutation, or C121S mutation, P124L mutation, or F129L mutation, and administering a combination of a PI3K inhibitor and MEK inhibitor if said mutation or deletion is present.

According to an embodiment of the invention, there is a method of treating a human suffering from V600 mutant melanoma comprising the steps of administering BRaf inhibitor Compound A or pharmaceutically acceptable salt thereof, determining if the melanoma tumor tissue of said human has at least one of an NRAS Q61 mutation, NRAS A146 mutation, MEK1 K59 deletion, MEK1 P387 mutation, or MEK1 Q56P mutation, or C121S mutation, P124L mutation, or F129L mutation and administering a combination of a PI3K inhibitor and MEK inhibitor if said mutation or deletion is present. Advantageously, the MEK inhibitor is Compound B or a pharmaceutically acceptable salt thereof.

According to an embodiment of the invention, there is a method of treating a human suffering from V600 mutant melanoma comprising the steps of administering BRaf inhibitor Compound A or pharmaceutically acceptable salt thereof, determining if the melanoma tumor tissue of said human has at least one of an NRAS Q61 mutation, NRAS A146 mutation, MEK1 K59 deletion, MEK1 P387 mutation, or MEK1 Q56P mutation, or C121S mutation, P124L mutation, or F129L mutation and administering a combination of PI3K inhibitor Compound C or pharmaceutically acceptable salt thereof and MEK inhibitor of Compound B or a pharmaceutically acceptable salt thereof.

According to an embodiment of the invention, there is a method of treating a human suffering from V600 mutant melanoma comprising the steps of administering N-{3-[5-(2-Amino-4-pyrimidinyl)-2-(1,1-dimethylethyl)-1,3-thiazol-4-yl]-2-fluorophenyl}-2,6-difluorobenzenesulfonamide methanesulfonate, determining if the melanoma tumor tissue of said human has at least one of an NRAS Q61 mutation, NRAS A146 mutation, MEK1 K59 deletion, or MEK1 P387 mutation, and administering a combination of 2,4-difluoro-N-{2-(methyloxy)-5-[4-(4-pyridazinyl)-6-quinolinyl]-3-pyridinyl}benzenesulfonamide and N-{3-[3-cyclopropyl-5-(2-fluoro-4-iodo-phenylamino)-6,8-dimethyl-2,4,7-trioxo-3,4,6,7-tetrahydro-2H-pyrido[4,3-d]pyrimidin-1-yl]phenyl}acetamide as a dimethyl sulfoxide solvate.

According to an embodiment of the invention, there is a method of treating a human suffering from V600 mutant melanoma comprising the steps of administering a first BRaf inhibitor, determining if the melanoma tumor tissue of said human has at least one of an NRAS Q61 mutation, NRAS A146 mutation, MEK1 K59 deletion, or MEK1 P387 mutation, or MEK1 Q56P mutation, or C121S mutation, P124L mutation, or F129L mutation, and administering a combination of a PI3K inhibitor and BRaf inhibitor if said mutation or deletion is present.

According to an embodiment of the invention, there is a method of treating a human suffering from V600 mutant melanoma comprising the steps of administering BRaf inhibitor Compound A or pharmaceutically acceptable salt thereof, determining if the melanoma tumor tissue of said human has at least one of an NRAS Q61 mutation, NRAS A146 mutation, MEK1 K59 deletion, MEK1 P387 mutation, or MEK1 Q56P mutation, or C121S mutation, P124L mutation, or F129L mutation and administering a combination of a PI3K inhibitor and BRaf inhibitor if said mutation or deletion is present. Advantageously, the PI3K inhibitor is Compound C or a pharmaceutically acceptable salt thereof.

According to an embodiment of the invention, there is a method of treating a human suffering from V600 mutant melanoma comprising the steps of administering BRaf inhibitor Compound A or pharmaceutically acceptable salt thereof, determining if the melanoma tumor tissue of said human has at least one of an NRAS Q61 mutation, NRAS A146 mutation, MEK1 K59 deletion, MEK1 P387 mutation, or MEK1 Q56P mutation, or C121S mutation, P124L mutation, or F129L mutation and administering a combination of PI3K inhibitor Compound C or pharmaceutically acceptable salt thereof and BRaf inhibitor of Compound A or a pharmaceutically acceptable salt thereof.

According to an embodiment of the invention, there is a method of treating a human suffering from V600 mutant melanoma comprising the steps of administering N-{3-[5-(2-Amino-4-pyrimidinyl)-2-(1,1-dimethylethyl)-1,3-thiazol-4-yl]-2-fluorophenyl}-2,6-difluorobenzenesulfonamide methanesulfonate, determining if the melanoma tumor tissue of said human has at least one of an NRAS Q61 mutation, NRAS A146 mutation, MEK1 K59 deletion, MEK1 P387 mutation, or MEK1 Q56P mutation, or C121S mutation, P124L mutation, or F129L mutation, and administering a combination of 2,4-difluoro-N-{2-(methyloxy)-5-[4-(4-pyridazinyl)-6-quinolinyl]-3-pyridinyl}benzenesulfonamide and 3-[5-(2-Amino-4-pyrimidinyl)-2-(1,1-dimethylethyl)-1,3-thiazol-4-yl]-2-fluorophenyl}-2,6-difluorobenzenesulfonamide methanesulfonate.

In further embodiments, any of the above methods of treating a human suffering from a V600 mutant melanoma comprises administering a BRaf inhibitor, such as Compound A or PLX4032 salts thereof, determining if the melanoma tissue of said human has at least one of an NRAS Q61 mutation, NRAS A146 mutation, MEK1 K59 deletion, MEK1 P387 mutation, or MEK1 Q56P mutation, or C121S mutation, P124L mutation, or F129L mutation, and administering a combination of a BRaf inhibitor, a MEK1 inhibitor, and a PI3K inhibitor. In yet a further embodiment, the combination of BRaf inhibitor, MEK1 inhibitor, and PI3K inhibitor is a combination of Compound A, Compound B, and Compound C.

The mutation that confers resistance to a particular therapy, such as Compound A or salts thereof or PLX4032 or salts thereof, may be present prior to or subsequent to the onset of cancer or prior to or subsequent to the onset of the therapy. In specific embodiments, the mutation that confers drug resistance is pre-existing at very low frequency prior to treatment. Under the selection pressure of drug treatment, the mutated clone outgrows other cells and results in drug resistance and rapid progression. In particular aspects of the invention, there is correlative analysis of the pre-existing mutation that confers resistance with likelihood of response or clinical duration of response. For example, the frequency of preexisting mutation(s) prior to treatment can serve as a tumor marker for prognosis and treatment decision-making.

### BRIEF DESCRIPTION OF THE DRAWINGS

FIG. 1: NRAS nucleotide and amino acid sequences (gene accession number NM_{—}002524.3). The locations for codons Q61 and A146 and their respective nucleotide sequences are indicated.

FIG. 2: MEK1 nucleotide and amino acid sequences (gene accession number NM_{—}002755.3). The locations for codons K59 and P387 and their respective nucleotide sequences are indicated.

FIG. 3: Melanoma cells having co-mutations of BRAF V600E and NRAS Q61 and/or A146 showed increased RAS GTPase activity.

FIG. 4: BRAF V600E melanoma cells having a co-mutation of NRAS Q61 and/or A146, optionally with further co-mutation of MEK1 P387 are resistant to BRAF inhibitor, Compound A, but sensitive to the combination of Compound A and Compound B (cloning assay).

FIG. 5: BRAF V600E melanoma cells having a co-mutation of NRAS Q61 and/or A146, optionally with further co-mutation of MEK1 P387 are resistant to BRAF inhibitor, Compound A, but sensitive to the combination of Compound B and Compound C (cloning assay).

FIG. 6 is an image of two BRAF inhibitors, Compound A and PLX4032, a MEK1 inhibitor Compound B, and a PI3K inhibitor Compound C.

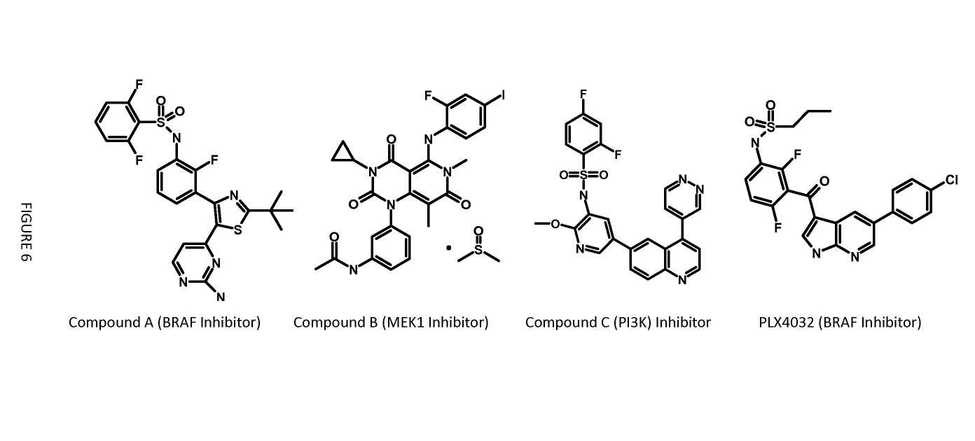

FIG. 7 is a graph of relative cell growth of various A375 F11 melanoma cell line clones in various concentrations of Compound A, a BRAF inhibitor.

FIG. 8 is a table summarizing cell growth inhibition assays of Compound A (BRAF inhibitor), Compound B (MEK inhibitor), Compound C (PI3K inhibitor) and their combination in A375 clones that acquired resistance to Compound A

FIG. 9 is a table summarizing cell growth inhibition assays of Compound A, Compound B, and Compound C, and their combination in YUSIT1 clones that acquired resistance to Compound A.

FIG. 10 is a is an image of a western blot comparing the signaling effects of treatment of four A375 clones with Compound A (BRAF inhibitor) as compared to the parental clone (A375).

FIG. 11 depicts a western blot of extracted and immunoprecipated with BRAF or CRAF antibodies, after treatment of the cells for 24 hours with Compound A, demonstrating that Compound A induces BRAF-CRAF heterodimer complex. parental A375, and 12R5-3 and 16R6-4 clones.

FIG. 12 is a graph of relative growth of cells that were treated with (NRAS clone 2 and 9 our without NRAS shRNA (Control clone 2 and 7) demonstrating that reducing expression of NRAS restores sensitivity to Compound A (BRAF inhibitor).

FIG. 13 is a graph of relative growth of cells that were treated with (NRAS clone 2 and 9 our without NRAS shRNA (Control clone 2 and 7) demonstrating that reducing expression of NRAS restores sensitivity to Compound B (MEK1 inhibitor).

FIG. 14 is a western blot of lysates of A375 clones treated with or without NRAS shRNA and with our without treatment with Compound A (BRAF inhibitor) showing that Compound A decreases phosphorylation of MEK and ERK.

FIG. 15 is a graph of relative growth of A375 cells expressing exogenous mutant NRAS protein and growing in the presence of various amounts of Compound A (BRAF inhibitor).

FIG. 16 is a graph of relative growth of A375 cells expressing exogenous NRAS mutants and after treatment with Compound B (MEK1 inhibitor) for three days. A375 PF11 (open circles), five A375 PF11 clones that express FLAG-NRASQ61K (closed symbols) and two A375 PF11 clones that express FLAG-NRASA146T after treatment with Compound B (MEK1 inhibitor) for three days.

FIG. 17 is an image of western blots of parental (A375) and A375 cells expressing exogeneous mutant NRASQ61K and NRASA146T clones demonstrating that cells expressing mutant NRAS do not show a decrease in phosphorylation of MEK, ERK, and S6P phosphorylation.

FIG. 18 is an image of modeled binding of Compound B (MEK1 inhibitor) to MEK1 protein.

FIG. 19 is a graph of relative growth of A375 cells expressing exogenous MEK1 mutants after treatment with various amounts of Compound A.

FIG. 20 is a graph of relative growth of A375 cells expressing exogenous MEK1 mutants after treatment with amounts of Compound B

FIG. 21 is a western blot of lysates of A375 cells expressing exogenous MEK1 mutants after treatment with Compound A.

FIG. 22 is a graph of relative growth of A375 parental and Compound A resistant cells treated with siRNA to knockout expression of MEK1 or MEK2.

FIG. 23 is an image of a western blot detecting expression or lack of expression in MEK1 and MEK2 siRNA treated A375 cells.

FIG. 24 depicts graphs of the relative growth curves of A375 PF11 (A.) and A375 PF11 derived clones that acquired resistance to Compound A 12R5-1 (B.), 12R5-3 (C.), 16R6-2 (D.), and 16R6-4 (E.) after treatment with Compound A, Compound B or their combination in a 10:1 constant molar to molar ratio for three days.

FIG. 25 depicts graphs of the relative growth curves of cells stably expressing NRAS Q61R (B.), NRAS Q61K (C., D.), and NRAS A146T (E., F.) or vehicle, after treatment with Compound A, Compound B, or their combination in a fixed 10:1 molar ratio for three days.

FIG. 26 depicts representative growth curves relative to vehicle of A375 PF11 transduced with Vector, MEK1 WT, MEK1 K59del, and MEK1 Q56P, after treatment with Compound A, Compound B, or their combination in a 10:1 fixed molar ratio combination for three days.

FIG. 27 depicts representative growth curves of A375 PF11 derived clones, 12R5-3, 16R6-4, and 12R5-5, that acquired resistance to Compound A after treatment with a dose response to Compound B in the presence of 0, 0.1, 0.3 and 1 μM Compound A for 3 days. A375 PF11 cells treated with various amounts of Compound B alone are shown (dashed line, open circle) for comparison.

FIG. 28 depicts representative images of the results of extended proliferation assays demonstrating that a combination of 1 uM Compound A (BRAF inhibitor) with 0.1 uM Compound B inhibited greater than 90% of cell growth in NRAS mutant clones.

FIG. 29 depicts western blots of various A375 mutants detecting phosphorylation of various RAF-MEK-ERK pathway molecules in the presence of Compound A, Compound B, or both.

FIG. 30 depicts western blots detecting phosphorylation of MEK, ERK, S6P, and AKT in cell lysates of A375 mutant cells treated with Compound C (PI3K inhibitor) with or without Compound A (BRAF inhibitor) or Compound B (MEK1 inhibitor).

FIG. 31 depicts representative images of the results of extended proliferation assays demonstrating enhanced growth inhibition with the combination of either Compound A (BRAF inhibitor) or Compound B (MEK1) in combination with Compound C (PI3K inhibitor).

FIG. 32 is a table summarizing cell growth inhibition assays of treatment with Compound A, Compound B, and Compound C, and their combination on cells expressing various MEK1 mutants.

FIG. 33 depicts graphs of relative growth curves of cells expressing various MEK1 mutants in the presence of Compound A or Compound B

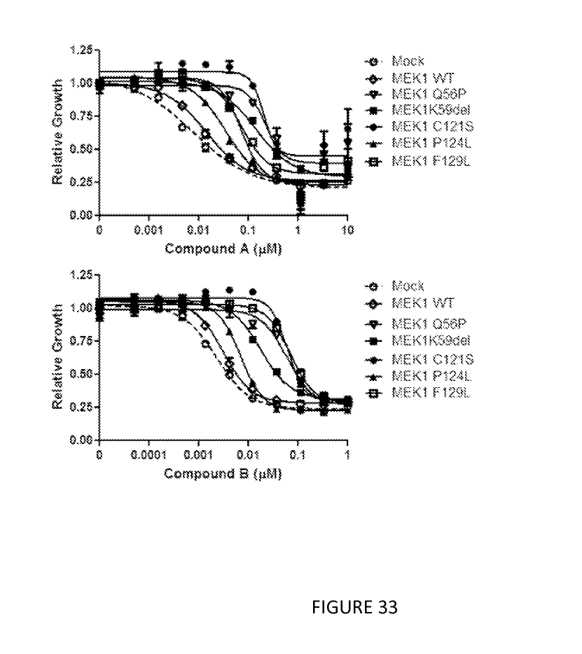

FIG. 34 is a table summarizing cell growth inhibition assays of YUSIT Q61K clones treated with Compound A, Compound B, Compound C, or the combinations shown.

FIG. 35 depicts graphs of relative growth of cells expressing various MEK1 mutants in the presence of Compound A.

### DETAILED DESCRIPTION OF THE INVENTION

### Mutations and Mutation Detection

The invention is based on the detection of one or a combination of NRAS Q61 mutation, NRAS A146 mutation, MEK1 K59 deletion, or MEK1 P387 mutation in humans suffering from V600 mutant melanoma.

The term “Ras protein” as used herein means any protein which is a member of the ras subfamily which is a subfamily of GTPases involved in cellular signaling. As is known in the art, activation of Ras causes cell growth, differentiation and survival. Ras proteins include, but are not limited to, H-ras, K-ras and N-ras.

As used herein “gene encoding a Ras protein” means any part of a gene or polynucleotide encoding any Ras protein. Included within the meaning of this term are exons encoding Ras. Gene encoding Ras proteins include but are not limited to genes encoding part or all of H-ras, K-ras and N-ras.

“NRAS protein” is an GTPase enzyme that in humans is encoded by NRAS (neuroblastoma RAS viral (v-ras) oncogene homolog) gene.

The term “mutant NRAS protein” and “N-ras mutation” refer to N-ras proteins having at least one mutation selected from Q61 R, Q61K, Q61H, Q61L, Q61N, Q61E, Q61P, A146T, A146P, or A146V. The mutant N-ras polypeptides may include, but are not limited to, allelic variants, splice variants, derivative variants, substitution variants, deletion variants, and/or insertion variants, fusion polypeptides, orthologs, and interspecies homologs. In certain embodiments, a mutant N-ras polypeptide includes additional residues at the C- or N-terminus, such as, but not limited to, leader sequence residues, targeting residues, amino terminal methionine residues, lysine residues, tag residues and/or fusion protein residues.

MEK1 protein, a dual specificity mitogen-activated protein kinase kinase 1, is an enzyme that in human is encoded by the MAP2K1 gene. The term “mutant MEK1 protein” and “MEK1 mutation” refer to MEK1 proteins having at least one mutation selected from 59delK and P387S or Q56P or C121S or P124L or F129L, or a MAP2K1 gene having at least one mutation selected from 175-177 AAG deletion or C1159T.

Additionally, mutant NRAS or MEK1 polypeptides include polypeptides or gene encoding a polypeptide in which part of all of the polypeptide or gene encoding the polypeptide is deleted or absent from the cell. For example, a NRAS protein may be produced by a cell in a truncated form. A deletion may mean the absence of all or part of a gene or protein encoded by a gene.

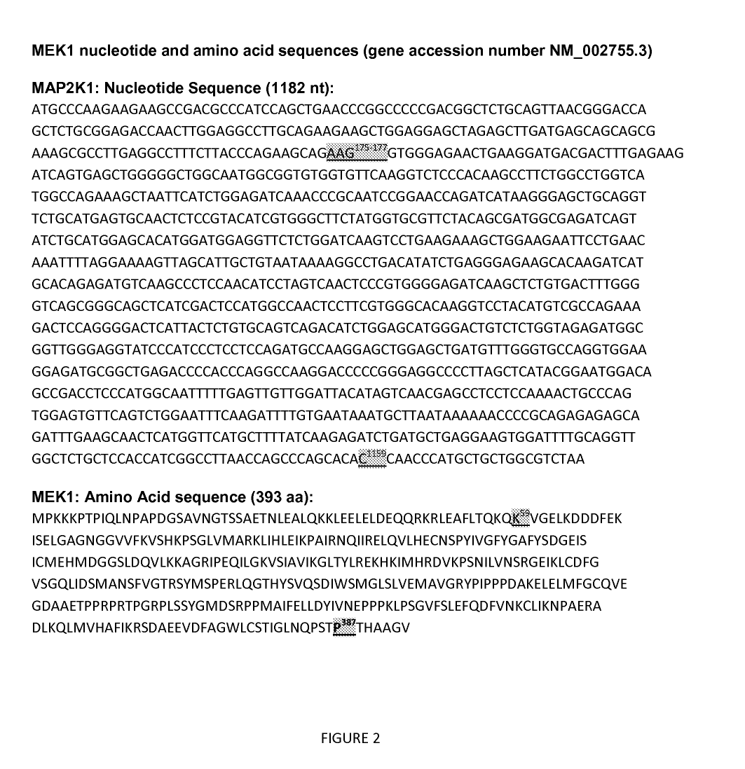

The term “V600 mutant B-raf protein” and similar terms refers to a B-raf polypeptide comprising a mutation at the V600 location. The B-raf protein may contain other mutations including, but are not limited to allelic variants, splice variants, derivative variants, substitution variants, deletion variants, and/or insertion variants, fusion polypeptides, orthologs, and interspecies homologs. In certain embodiments, a mutant B-raf polypeptide includes additional residues at the C- or N-terminus, such as, but not limited to, leader sequence residues, targeting residues, amino terminal methionine residues, lysine residues, tag residues and/or fusion protein residues. Certain V600 B-raf mutants include but are not limited to BRAF having an amino acid substitution selected from V600E, V600D, V600K, V600R, and V600L. See, for example, FIG. 2 of Halilovic and Solvit (2008) Current Opinion in Pharmacology 8:419-26.

The term “wild type” as is understood in the art refers to a polypeptide or polynucleotide sequence that occurs in a native population without genetic modification. As is also understood in the art, a “mutant” includes a polypeptide or polynucleotide sequence having at least one modification to an amino acid or nucleic acid compared to the corresponding amino acid or nucleic acid found in a wild type polypeptide or polynucleotide, respectively. Included in the term mutant is Single Nucleotide Polymorphism (SNP) where a single base pair distinction exists in the sequence of a nucleic acid strand compared to the most prevalently found (wild type) nucleic acid strand.

As used herein, “genotyping” a cell including a tumor cell from a subject (or DNA or other biological sample) for a mutation or a polymorphic allele of a gene(s) means detecting which allelic or polymorphic form(s) and/or wild type or somatically mutated form(s) of the gene(s) or gene expression products (e.g., hnRNA, mRNA or protein) are present or absent in a subject (or a sample). Related RNA or protein expressed from such gene may also be used to detect polymorphic variation. For purposes of the present invention, “genotyping” includes the determination of somatic as well as genotypic mutations from a sample. As used herein, an allele may be ‘detected’ when other possible allelic variants have been ruled out; e.g., where a specified nucleic acid position is found to be neither adenine (A), thymine (T) or cytosine (C), it can be concluded that guanine (G) is present at that position (i.e., G is ‘detected’ or ‘diagnosed’ in a subject). Sequence variations may be detected directly (by, e.g. sequencing, for example, EST sequencing or partial or full genome sequencing) or indirectly (e.g., by restriction fragment length polymorphism analysis, or detection of the hybridization of a probe of known sequence, or reference strand conformation polymorphism), or by using other known methods.

The sequence of any nucleic acid including a gene or PCR product or a fragment or portion thereof may be sequenced by any method known in the art (e.g., chemical sequencing or enzymatic sequencing). “Chemical sequencing” of DNA may denote methods such as that of Maxam and Gilbert (1977) (Proc. Natl. Acad. Sci. USA 74:560), in which DNA is randomly cleaved using individual base-specific reactions. “Enzymatic sequencing” of DNA may denote methods such as that of Sanger (Sanger, et al., (1977) Proc. Natl. Acad. Sci. USA 74:5463).

Conventional molecular biology, microbiology, and recombinant DNA techniques including sequencing techniques are well known among those skilled in the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook, et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984).

The Peptide Nucleic Acid (PNA) affinity assay is a derivative of traditional hybridization assays (Nielsen et al., Science 254:1497-1500 (1991); Egholm et al., J. Am. Chem. Soc. 114:1895-1897 (1992); James et al., Protein Science 3:1347-1350 (1994)). PNAs are structural DNA mimics that follow Watson-Crick base pairing rules, and are used in standard DNA hybridization assays. PNAs display greater specificity in hybridization assays because a PNA/DNA mismatch is more destabilizing than a DNA/DNA mismatch and complementary PNA/DNA strands form stronger bonds than complementary DNA/DNA strands.

DNA microarrays have been developed to detect genetic variations and polymorphisms (Teton et al., Science 289:1757-60, 2000; Lockhart et al., Nature 405:827-836 (2000); Gerhold et al., Trends in Biochemical Sciences 24:168-73 (1999); Wallace, R. W., Molecular Medicine Today 3:384-89 (1997); Blanchard and Hood, Nature Biotechnology 149:1649 (1996)). DNA microarrays are fabricated by high-speed robotics, on glass or nylon substrates, and contain DNA fragments with known identities (“the probe”). The microarrays are used for matching known and unknown DNA fragments (“the target”) based on traditional base-pairing rules.

The term “at least one mutation” in a polypeptide or a gene encoding a polypeptide and grammatical variations thereof means a polypeptide or gene encoding a polypeptide having one or more allelic variants, splice variants, derivative variants, substitution variants, deletion variants, truncation variants, and/or insertion variants, fusion polypeptides, orthologs, and/or interspecies homologs. By way of example, at least one mutation of a NRAS protein would include a NRAS protein in which part of all of the sequence of a polypeptide or gene encoding the NRAS protein is absent or not expressed in the cell for at least one NRAS protein produced in the cell. For example, a NRAS protein may be produced by a cell in a truncated form and the sequence of the truncated form may be wild type over the sequence of the truncate. A deletion may mean the absence of all or part of a gene or protein encoded by a gene. Additionally, some of a protein expressed in or encoded by a cell may be mutated while other copies of the same protein produced in the same cell may be wild type. By way of another example a mutation in a NRAS protein would include a NRAS protein having one or more amino acid differences in its amino acid sequence compared with wild type of the same NRAS protein.

As used herein “genetic abnormality” is meant a deletion, substitution, addition, translocation, amplification and the like relative to the normal native nucleic acid content of a cell of a subject.

The terms “polypeptide” and “protein” are used interchangeably and are used herein as a generic term to refer to native protein, fragments, peptides, or analogs of a polypeptide sequence. Hence, native protein, fragments, and analogs are species of the polypeptide genus.

The terminology “X#Y” in the context of a mutation in a polypeptide sequence is art-recognized, where “#” indicates the location of the mutation in terms of the amino acid number of the polypeptide, “X” indicates the amino acid found at that position in the wild-type amino acid sequence, and “Y” indicates the mutant amino acid at that position. For example, the notation “G12S” with reference to the K-ras polypeptide indicates that there is a glycine at amino acid number 12 of the wild-type K-ras sequence, and that glycine is replaced with a serine in the mutant K-ras sequence.

As used herein the term “amplification” and grammatical variations thereof refers to the presence of one or more extra gene copies in a chromosome complement. In certain embodiments a gene encoding a Ras protein may be amplified in a cell. Amplification of the HER2 gene has been correlated with certain types of cancer. Amplification of the HER2 gene has been found in human salivary gland and gastric tumor-derived cell lines, gastric and colon adenocarcinomas, and mammary gland adenocarcinomas. Semba et al., Proc. Natl. Acad. Sci. USA, 82:6497-6501 (1985); Yokota et al., Oncogene, 2:283-287 (1988); Zhou et al., Cancer Res., 47:6123-6125 (1987); King et al., Science, 229:974-976 (1985); Kraus et al., EMBO J., 6:605-610 (1987); van de Vijver et al., Mol. Cell. Biol., 7:2019-2023 (1987); Yamamoto et al., Nature, 319:230-234 (1986).

As used herein “overexpressed” and “overexpression” of a protein or polypeptide and grammatical variations thereof means that a given cell produces an increased number of a certain protein relative to a normal cell. By way of example, a ras protein may be overexpressed by a tumor cell relative to a non-tumor cell. Additionally, a mutant ras protein may be overexpressed compared to wild type ras protein in a cell. As is understood in the art, expression levels of a polypeptide in a cell can be normalized to a housekeeping gene such as actin. In some instances, a certain polypeptide may be underexpressed in a tumor cell compared with a non-tumor cell.

As used herein “nucleic acid necessary for expression of at least one gene product” refers to a nucleic acid sequence that encodes any portion of a gene and/or is operably linked to a nucleic acid encoding a gene product but does not necessarily comprise encoding sequence. By way of example, a nucleic acid sequence necessary for the expression of at least one gene product includes, but is not limited to, enhancers, promoters, regulatory sequences, start codons, stop codons, polyadenylation sequences, and/or encoding sequences. Expression levels of a polypeptide in a particular cell can be effected by, but not limited to, mutations, deletions and/or substitutions of various regulatory elements and/or non-encoding sequence in the cell genome.

The term “polynucleotide” as referred to herein means a polymeric form of nucleotides of at least 10 bases in length, either ribonucleotides or deoxynucleotides or a modified form of either type of nucleotide. The term includes single and double stranded forms of DNA.

The term “oligonucleotide” referred to herein includes naturally occurring and modified nucleotides linked together by naturally occurring, and non-naturally occurring oligonucleotide linkages. Oligonucleotides are a polynucleotide subset generally comprising a length of 200 bases or fewer. Preferably oligonucleotides are 10 to 60 bases in length and most preferably 12, 13, 14, 15, 16, 17, 18, 19, or 20 to 40 bases in length. Oligonucleotides are usually single stranded, e.g. for probes, although oligonucleotides may be double stranded, e.g. for use in the construction of a gene mutant. Oligonucleotides can be either sense or antisense oligonucleotides.

An oligonucleotide probe, or probe, is a nucleic acid molecule which typically ranges in size from about 8 nucleotides to several hundred nucleotides in length. Such a molecule is typically used to identify a target nucleic acid sequence in a sample by hybridizing to such target nucleic acid sequence under stringent hybridization conditions. Hybridization conditions have been described in detail above.

PCR primers are also nucleic acid sequences, although PCR primers are typically oligonucleotides of fairly short length which are used in polymerase chain reactions. PCR primers and hybridization probes can readily be developed and produced by those of skill in the art, using sequence information from the target sequence. (See, for example, Sambrook et al., supra or Glick et al., supra).

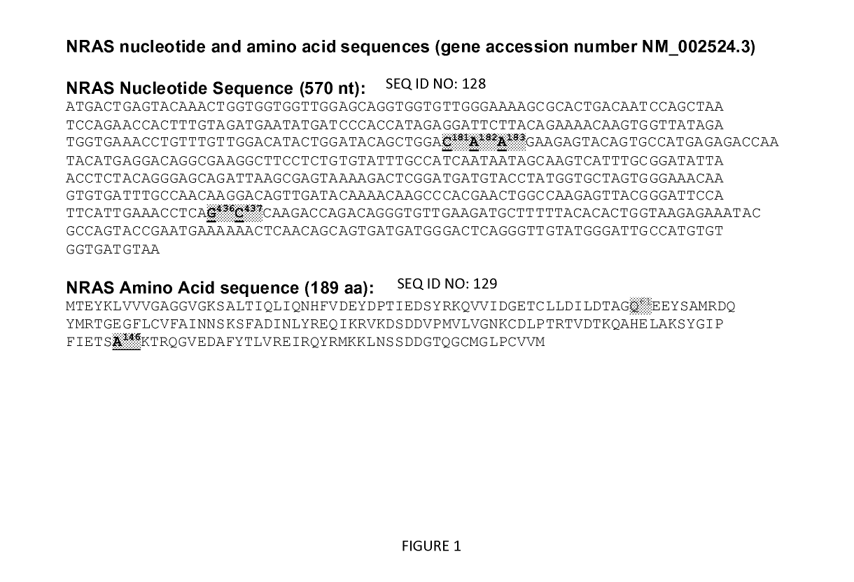

As is known in the art, several PCR and/or sequencing based methods are known for use in detecting NRAS, MEK1 and BRAF mutations and are presented in several research articles and US patents including, but not limited to, Brose, et al. Cancer Research 62:6997-7000 (2002), Solit et al, Cancer Research 70(14): 5901-5911 (1010), Xu, et al. Cancer research 63:4561-4567 (2003), as well as U.S. Pat. No. 7,745,128, and several commercially available kits (see Dxs Diagnostic Innovations, Applied Biosystems, and Quest diagnostics). In general, primers and probes for sequencing or PCR can be designed based on the codon mutations of Q61K (C181A), Q61H (A183C or A183T), Q61R (A182G), Q61L (A182T), Q61N(C181A, A183C), Q61E (C181G), Q61P (A182C), A146T (G436A), A146P (G436C) and A146V (C437G) and the sequences for NRAS with gene accession number NM_{—}002524.3s (FIG. 1), mutations of MEK1 deletion at codon 59 (AAG deletion) or P387S(C1159T) and the sequences for MEK1 with gene accession number NM_{—}002755.3 (FIG. 2).

Cancers that are either wild type or mutant for Ras/Raf are identified by known methods. For example, wild type or mutant Ras/Raf tumor cells can be identified by DNA amplification and sequencing techniques, DNA and RNA detection techniques, including, but not limited to Northern and Southern blot, respectively, and/or various biochip and array technologies. Wild type and mutant polypeptides can be detected by a variety of techniques including, but not limited to immunodiagnostic techniques such as ELISA, Western blot or immunocyto chemistry.

### Melanoma

Suitably, the present invention relates to a method for treating or lessening the severity of BRAF V600 mutant melanoma, including subtypes of melanoma such as but not limited to superficial spreading melanoma, nodular melanoma, lentigo maligna melanoma, acral lentiginous melanoma, and choroidal intraocular melanoma, ocular intraocular melanoma, and uveal intraocular melanoma.

As used herein, the terms “cancer,” “neoplasm,” and “tumor,” are used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient.

As is well known in the art, tumors may metastasize from a first or primary locus of tumor to one or more other body tissues or sites. In particular, metastases to the central nervous system (i.e., secondary CNS tumors), and particularly the brain (i.e., brain metastases), are well documented for tumors and cancers, such as breast, lung, melanoma, renal and colorectal. As used herein, reference to uses or methods for treatment or treatments for a “neoplasm,” “tumor” or “cancer” in a subject includes use for and treatment of the primary neoplasm, tumor or cancer, and where appropriate, also the use for and treatment of metastases (i.e., metastatic tumor growth) as well.

Therapies that Overcome BRaf Resistance

### BRAF+MEK

It has been found that V600 mutant melanoma that exhibits NRAS Q61 and/or A146 mutations as described above, and therefore tends to be resistant to treatment with a BRaf inhibitor, is nonetheless treatable with a small number of select drug combinations of varying inhibitor types.

Surprisingly, V600 mutant melanoma that exhibits NRAS Q61 and/or A146 mutation is treatable with a combination of BRaf inhibitor and MEK inhibitor such that cell growth inhibition exceeds the additive effect of the individual agents.

By way of example, V600 mutant melanoma that exhibits NRAS Q61 and/or A146 mutation has been shown to respond with renewed cell growth inhibition upon treatment with the combination of BRaf inhibitor Compound A or pharmaceutically acceptable salts thereof and the MEK inhibitor for formula (II) (hereinafter “Compound B”) or pharmaceutically acceptable salts or solvates thereof:

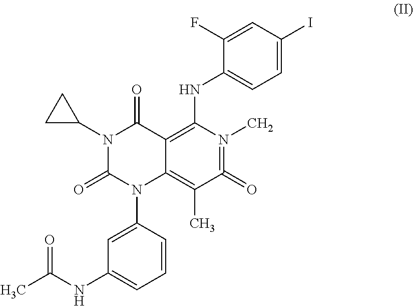

Compound B is also known as USAN name trametinib.

As shown in the Examples section below, the combination of BRaf inhibitor Compound A or pharmaceutically acceptable salts or solvates thereof and the MEK inhibitor for formula (II) or pharmaceutically acceptable salts or solvates thereof inhibit cellular proliferation of tumor cells that have previously been exposed to BRaf inhibitor Compound A and which have developed resistance to BRaf inhibitor Compound A, as demonstrated by the examples and as correlated to the exhibition of NRAS Q61 and/or A146 mutation. The combination provides superior inhibition of the NRAS mutated cells over either of the single agents.

According to an embodiment of the invention, there is a method of treating a human suffering from V600 mutant melanoma comprising the steps of administering a first BRaf inhibitor, determining if the melanoma tumor tissue of said human has an NRAS Q61 and/or A146 mutation, and administering a combination of second BRaf inhibitor and MEK inhibitor if said NRAS Q61 and/or A146 mutation is present. The first and second BRaf inhibitors may but are not required to be the same.

According to an embodiment of the invention, there is a method of treating a human suffering from V600 mutant melanoma comprising the steps of administering BRaf inhibitor Compound A or pharmaceutically acceptable salt thereof, determining if the melanoma tumor tissue of said human has an NRAS Q61 and/or A146 mutation, and administering a combination of a second BRaf inhibitor and MEK inhibitor if said NRAS Q61 and/or A146 mutation is present. Advantageously, the second BRaf inhibitor is BRaf inhibitor Compound A or pharmaceutically acceptable salt thereof. Advantageously, the MEK inhibitor is Compound B or a pharmaceutically acceptable salt thereof.

According to an embodiment of the invention, there is a method of treating a human suffering from V600 mutant melanoma comprising the steps of administering BRaf inhibitor Compound A or pharmaceutically acceptable salt thereof, determining if the melanoma tumor tissue of said human has an NRAS Q61 and/or A146 mutation, and administering a combination of BRaf inhibitor Compound A or pharmaceutically acceptable salt thereof and MEK inhibitor of Compound B or a pharmaceutically acceptable salt thereof.

It has been found that V600 mutant melanoma that exhibits a MEK1 K59 deletion, a MEK1 P387 mutation, a MEK1 Q56 mutation, a MEK1 C121 mutation, a MEK1 P124 mutation, or a MEK1 F129 mutation, as described above, and therefore tends to be resistant to treatment with a BRaf inhibitor, is nonetheless treatable with a small number of select drug combinations of varying inhibitor types.

Surprisingly, V600 mutant melanoma that exhibits a MEK1 K59 deletion, a MEK1 P387 mutation, a MEK1 Q56 mutation, a MEK1 C121 mutation, a MEK1 P124 mutation, or a MEK1 F129 mutation is treatable with a combination of BRaf inhibitor and MEK inhibitor such that cell growth inhibition exceeds the additive effect of the individual agents.

By way of example, V600 mutant melanoma that exhibits a MEK1 K59 deletion, a MEK1 P387 mutation, a MEK1 Q56 mutation, a MEK1 C121 mutation, a MEK1 P124 mutation, or a MEK1 F129 mutation has been shown to respond with renewed cell growth inhibition upon treatment with the combination of BRaf inhibitor Compound A or pharmaceutically acceptable salts thereof and the MEK inhibitor of Compound B or pharmaceutically acceptable salts thereof.

As shown in the Examples section below, the combination of BRaf inhibitor Compound A or pharmaceutically acceptable salts thereof and the MEK inhibitor of Compound B or pharmaceutically acceptable salts inhibit cellular proliferation of tumor cells that have previously been exposed to BRaf inhibitor Compound A and which have developed resistance to BRaf inhibitor Compound A, as demonstrated by the examples and as correlated to the exhibition of a MEK1 K59 deletion, a MEK1 P387 mutation, a MEK1 Q56 mutation, a MEK1 C121 mutation, a MEK1 P124 mutation, or a MEK1 F129 mutation. The combination provides superior inhibition of the MEK1 mutated cells over either of the single agents.

According to an embodiment of the invention, there is a method of treating a human suffering from V600 mutant melanoma comprising the steps of administering a first BRaf inhibitor, determining if the melanoma tumor tissue of said human has a MEK1 K59 deletion or a MEK1 P387 mutation, and administering a combination of second BRaf inhibitor and MEK inhibitor if said MEK1 K59 deletion, a MEK1 P387 mutation, a MEK1 Q56 mutation, a MEK1 C121 mutation, a MEK1 P124 mutation, or a MEK1 F129 mutation is present. The first and second BRaf inhibitors may but are not required to be the same.

According to an embodiment of the invention, there is a method of treating a human suffering from V600 mutant melanoma comprising the steps of administering BRaf inhibitor Compound A or pharmaceutically acceptable salt thereof, determining if the melanoma tumor tissue of said human has a MEK1 K59 deletion, a MEK1 P387 mutation, a MEK1 Q56 mutation, a MEK1 C121 mutation, a MEK1 P124 mutation, or a MEK1 F129 mutation, and administering a combination of a second BRaf inhibitor and MEK inhibitor if said MEK1 K59 deletion, a MEK1 P387 mutation, a MEK1 Q56 mutation, a MEK1 C121 mutation, a MEK1 P124 mutation, or a MEK1 F129 mutation is present. Advantageously, the second BRaf inhibitor is BRaf inhibitor Compound A or pharmaceutically acceptable salt thereof. Advantageously, the MEK inhibitor is Compound B or a pharmaceutically acceptable salt or solvate thereof.

According to an embodiment of the invention, there is a method of treating a human suffering from V600 mutant melanoma comprising the steps of administering BRaf inhibitor Compound A or pharmaceutically acceptable salt thereof, determining if the melanoma tumor tissue of said human has a MEK1 K59 deletion, a MEK1 P387 mutation, a MEK1 Q56 mutation, a MEK1 C121 mutation, a MEK1 P124 mutation, or a MEK1 F129 mutation, and administering a combination of BRaf inhibitor Compound A or pharmaceutically acceptable salt thereof and MEK inhibitor of Compound B or a pharmaceutically acceptable salt or solvate thereof.

According to an embodiment of the invention, there is a method of treating a human suffering from V600 mutant melanoma comprising the steps of administering N-{3-[5-(2-Amino-4-pyrimidinyl)-2-(1,1-dimethylethyl)-1,3-thiazol-4-yl]-2-fluorophenyl}-2,6-difluorobenzenesulfonamide methanesulfonate, determining if the melanoma tumor tissue of said human has an NRAS Q61 and/or A146 mutation, and administering a combination of N-{3-[5-(2-Amino-4-pyrimidinyl)-2-(1,1-dimethylethyl)-1,3-thiazol-4-yl]-2-fluorophenyl}-2,6-difluorobenzenesulfonamide methanesulfonate and N-{3-[3-cyclopropyl-5-(2-fluoro-4-iodo-phenylamino)-6,8-dimethyl-2,4,7-trioxo-3,4,6,7-tetrahydro-2H-pyrido[4,3-d]pyrimidin-1-yl]phenyl}acetamide as a dimethyl sulfoxide solvate.

According to an embodiment of the invention, there is a method of treating a human suffering from V600 mutant melanoma comprising the steps of administering N-{3-[5-(2-Amino-4-pyrimidinyl)-2-(1,1-dimethylethyl)-1,3-thiazol-4-yl]-2-fluorophenyl}-2,6-difluorobenzenesulfonamide methanesulfonate, determining if the melanoma tumor tissue of said human has a MEK1 K59 deletion, a MEK1 P387 mutation, a MEK1 Q56 mutation, a MEK1 C121 mutation, a MEK1 P124 mutation, or a MEK1 F129 mutation, and administering a combination of N-{3-[5-(2-Amino-4-pyrimidinyl)-2-(1,1-dimethylethyl)-1,3-thiazol-4-yl]-2-fluorophenyl}-2,6-difluorobenzenesulfonamide methanesulfonate and N-{3-[3-cyclopropyl-5-(2-fluoro-4-iodo-phenylamino)-6,8-dimethyl-2,4,7-trioxo-3,4,6,7-tetrahydro-2H-pyrido[4,3-d]pyrimidin-1-yl]phenyl}acetamide as a dimethyl sulfoxide solvate.

For the avoidance of confusion, it is asserted that any of the above described deletions or mutations in V600 mutated BRAF melanoma, i.e. any one of: the NRAS mutation at codon 61, the NRAS mutation at codon 146, the MEK1 deletion at codon 59, or the MEK1 mutation at codon 387, 56, 121, 124, or 129, correlates with a decreased response or resistance to treatment of said melanoma with a BRAF inhibitor, e.g. the inhibitor of Compound A. Likewise, it is asserted that the combined occurrence of any of the above described deletions or mutations in V600 mutated BRAF melanoma correlates with a decreased response or resistance to treatment of said melanoma with a BRAF inhibitor, e.g. the inhibitor of Compound A. Further, it is asserted that the above described deletions or mutations in V600 mutated BRAF melanoma, occurring alone or in any combination, correlates with an increased likelihood of efficacious treatment of said melanoma with the combination of Compound A and Compound B as described herein.

Compound A is disclosed and claimed, along with pharmaceutically acceptable salts thereof, as being useful as an inhibitor of BRaf activity, particularly in the treatment of cancer, in PCT patent publication WO2009/137391, hereby incorporated by reference. Compound A is embodied by Examples 58a through 58e of the application.

Compound B is disclosed and claimed, along with pharmaceutically acceptable salts thereof, and also as solvates thereof, as being useful as an inhibitor of MEK activity, particularly in treatment of cancer, in WO 2005/121142, which is incorporated by reference herein. Compound B is the compound of Example 4-1. Compound A can be prepared as described in WO 2005/121142, which is incorporated by reference herein. Suitably, Compound B is in the form of a dimethyl sulfoxide solvate. Suitably, Compound B is in the form of a sodium salt. Suitably, Compound B is in the form of a solvate selected from: hydrate, acetic acid, ethanol, nitromethane, chlorobenzene, 1-pentancol, isopropyl alcohol, ethylene glycol and 3-methyl-1-butanol. These solvates and salt forms can be prepared by one of skill in the art from the description in WO 2005/121142, which is incorporated by reference herein.

The combined administration of Compound A and Compound B is disclosed, along with suitable formulations and dosing, in PCT/US2010/052808, filed Oct. 15, 2010, which teachings are incorporated by reference and which may be relied on as instructive as to the various possible methods of administering the combination.

Compounds A and B may be presented, separately or both, as solvates. As used herein, the term “solvate” refers to a complex of variable stoichiometry formed by a solute (in this invention, compounds of formula (I) or (II) or a salt thereof and a solvent. Such solvents for the purpose of the invention may not interfere with the biological activity of the solute. Examples of suitable solvents include, but are not limited to, water, methanol, dimethylsulforide. ethanol and acetic acid. In one embodiment, the solvent used is a pharmaceutically acceptable solvent. Examples of suitable pharmaceutically acceptable solvents include, without limitation, water, ethanol and acetic acid. In another embodiment, the solvent used is water.

Compounds A and B may have the ability to crystallize in more than one form, a characteristic, which is known polymorphism, and it is understood that such polymorphic forms (“polymorphs”) are within the scope of Compounds A and B. Polymorphism generally can occur as a response to changes in temperature or pressure or both and can also result from variations in the crystallization process. Polymorphs can be distinguished by various physical characteristics known in the art such as x-ray diffraction patterns, solubility, and melting point.

Suitably, the amount of Compound A (based on weight of unsalted/unsolvated amount) administered as part of the combination according to the present invention will be an amount selected from about 10 mg to about 600 mg. Suitably, the selected amount of Compound A is administered from 1 to 4 times a day. Suitably, Compound A is administered at an amount of 150 mg twice a day.

Suitably, the amount of Compound B (based on weight of unsalted/unsolvated amount) administered as part of the combination according to the present invention will be an amount selected from about 0.125 mg to about 10 mg.

### MEK+PI3K

Surprisingly, V600 mutant melanoma that exhibits NRAS Q61 and/or A146 mutation is treatable with a combination of PI3K inhibitor and MEK inhibitor such that cell growth inhibition exceeds the additive effect of the individual agents.

By way of example, V600 mutant melanoma that exhibits NRAS Q61 and/or A146 mutation has been shown to respond with renewed cell growth inhibition upon treatment with the combination of the PI3K inhibitor of formula (III)

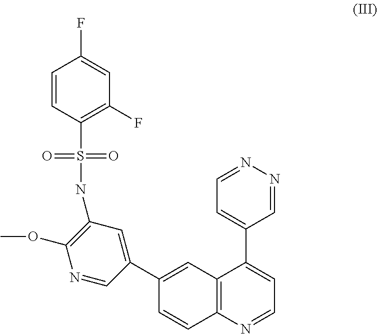 [SMILES: COc1ncc(-c2ccc3nccc(-c4ccnnc4)c3c2)cc1NS(=O)(=O)c1ccc(F)cc1F]

(hereinafter referred to as “Compound C”) or pharmaceutically acceptable salts thereof and the MEK inhibitor Compound B or pharmaceutically acceptable salts thereof.

As shown in the Examples section below, the combination of PI3K inhibitor Compound C or pharmaceutically acceptable salts thereof and the MEK inhibitor Compound B or pharmaceutically acceptable salts thereof inhibit cellular proliferation of tumor cells that have previously been exposed to BRaf inhibitor Compound A and which have developed resistance to BRaf inhibitor Compound A, as demonstrated by the examples and as correlated to the exhibition of NRAS Q61 and/or A146 mutation. The combination provides superior inhibition of the NRAS mutated cells over either of the single agents.

According to an embodiment of the invention, there is a method of treating a human suffering from V600 mutant melanoma comprising the steps of administering a first BRaf inhibitor, determining if the melanoma tumor tissue of said human has an NRAS Q61 and/or A146 mutation, and administering a combination of a PI3K inhibitor and MEK inhibitor if said NRAS Q61 and/or A146 mutation is present.

According to an embodiment of the invention, there is a method of treating a human suffering from V600 mutant melanoma comprising the steps of administering BRaf inhibitor Compound A or pharmaceutically acceptable salt thereof, determining if the melanoma tumor tissue of said human has an NRAS Q61 and/or A146 mutation, and administering a combination of a PI3K inhibitor and MEK inhibitor if said NRAS Q61 and/or A146 mutation is present. Advantageously, the MEK inhibitor is Compound B or a pharmaceutically acceptable salt thereof.

According to an embodiment of the invention, there is a method of treating a human suffering from V600 mutant melanoma comprising the steps of administering BRaf inhibitor Compound A or pharmaceutically acceptable salt thereof, determining if the melanoma tumor tissue of said human has an NRAS Q61 and/or A146 mutation, and administering a combination of PI3K inhibitor Compound C or pharmaceutically acceptable salt thereof and MEK inhibitor of Compound B or a pharmaceutically acceptable salt thereof.

Surprisingly, V600 mutant melanoma that exhibits a MEK1 K59 deletion, a MEK1 P387 mutation, a MEK1 Q56 mutation, a MEK1 C121 mutation, a MEK1 P124 mutation, or a MEK1 F129 mutation is treatable with a combination of PI3K/mTOR inhibitor and MEK inhibitor such that cell growth inhibition exceeds the additive effect of the individual agents.

By way of example, V600 mutant melanoma that exhibits a MEK1 K59 deletion, a MEK1 P387 mutation, a MEK1 Q56 mutation, a MEK1 C121 mutation, a MEK1 P124 mutation, or a MEK1 F129 mutation has been shown to respond with renewed cell growth inhibition upon treatment with the combination of the PI3K inhibitor of Compound C or pharmaceutically acceptable salts thereof and the MEK inhibitor Compound B or pharmaceutically acceptable salts thereof.

As shown in the Examples section below, the combination of PI3K inhibitor Compound C or pharmaceutically acceptable salts thereof and the MEK inhibitor Compound B or pharmaceutically acceptable salts thereof inhibit cellular proliferation of tumor cells that have previously been exposed to BRaf inhibitor Compound A and which have developed resistance to BRaf inhibitor Compound A, as demonstrated by the examples and as correlated to the exhibition of a MEK1 K59 deletion, a MEK1 P387 mutation, a MEK1 Q56 mutation, a MEK1 C121 mutation, a MEK1 P124 mutation, or a MEK1 F129 mutation. The combination provides superior inhibition of the MEK1 mutated cells over either of the single agents.

According to an embodiment of the invention, there is a method of treating a human suffering from V600 mutant melanoma comprising the steps of administering a first BRaf inhibitor, determining if the melanoma tumor tissue of said human has a MEK1 K59 deletion, a MEK1 P387 mutation, a MEK1 Q56 mutation, a MEK1 C121 mutation, a MEK1 P124 mutation, or a MEK1 F129 mutation, and administering a combination of a PI3K inhibitor and MEK inhibitor if said a MEK1 K59 deletion, a MEK1 P387 mutation, a MEK1 Q56 mutation, a MEK1 C121 mutation, a MEK1 P124 mutation, or a MEK1 F129 mutation is present.

According to an embodiment of the invention, there is a method of treating a human suffering from V600 mutant melanoma comprising the steps of administering BRaf inhibitor Compound A or pharmaceutically acceptable salt thereof, determining if the melanoma tumor tissue of said human has a MEK1 K59 deletion, a MEK1 P387 mutation, a MEK1 Q56 mutation, a MEK1 C121 mutation, a MEK1 P124 mutation, or a MEK1 F129 mutation, and administering a combination of a PI3K inhibitor and MEK inhibitor if said a MEK1 K59 deletion, a MEK1 P387 mutation, a MEK1 Q56 mutation, a MEK1 C121 mutation, a MEK1 P124 mutation, or a MEK1 F129 mutation is present. Advantageously, the MEK inhibitor is Compound B or a pharmaceutically acceptable salt thereof.

According to an embodiment of the invention, there is a method of treating a human suffering from V600 mutant melanoma comprising the steps of administering BRaf inhibitor Compound A or pharmaceutically acceptable salt thereof, determining if the melanoma tumor tissue of said human has a MEK1 K59 deletion, a MEK1 P387 mutation, a MEK1 Q56 mutation, a MEK1 C121 mutation, a MEK1 P124 mutation, or a MEK1 F129 mutation, and administering a combination of PI3K inhibitor Compound C or pharmaceutically acceptable salt thereof and MEK inhibitor of Compound B or a pharmaceutically acceptable salt thereof.

According to an embodiment of the invention, there is a method of treating a human suffering from V600 mutant melanoma comprising the steps of administering N-{3-[5-(2-Amino-4-pyrimidinyl)-2-(1,1-dimethylethyl)-1,3-thiazol-4-yl]-2-fluorophenyl}-2,6-difluorobenzenesulfonamide methanesulfonate, determining if the melanoma tumor tissue of said human has an NRAS Q61 and/or A146 mutation, and administering a combination of 2,4-difluoro-N-{2-(methyloxy)-5-[4-(4-pyridazinyl)-6-quinolinyl]-3-pyridinyl}benzenesulfonamide and N-{3-[3-cyclopropyl-5-(2-fluoro-4-iodo-phenylamino)-6,8-dimethyl-2,4,7-trioxo-3,4,6,7-tetrahydro-2H-pyrido[4,3-d]pyrimidin-1-yl]phenyl}acetamide as a dimethyl sulfoxide solvate.

According to an embodiment of the invention, there is a method of treating a human suffering from V600 mutant melanoma comprising the steps of administering N-{3-[5-(2-Amino-4-pyrimidinyl)-2-(1,1-dimethylethyl)-1,3-thiazol-4-yl]-2-fluorophenyl}-2,6-difluorobenzenesulfonamide methanesulfonate, determining if the melanoma tumor tissue of said human has a MEK1 K59 deletion, a MEK1 P387 mutation, a MEK1 Q56 mutation, a MEK1 C121 mutation, a MEK1 P124 mutation, or a MEK1 F129 mutation, and administering a combination of 2,4-difluoro-N-{2-(methyloxy)-5-[4-(4-pyridazinyl)-6-quinolinyl]-3-pyridinyl}benzenesulfonamide and N-{3-[3-cyclopropyl-5-(2-fluoro-4-iodo-phenylamino)-6,8-dimethyl-2,4,7-trioxo-3,4,6,7-tetrahydro-2H-pyrido[4,3-d]pyrimidin-1-yl]phenyl}acetamide as a dimethyl sulfoxide solvate.

For the avoidance of confusion, it is asserted that any of the above described deletions or mutations in V600 mutated BRAF melanoma, i.e. any one of: the NRAS mutation at codon 61, the NRAS mutation at codon 146, the MEK1 deletion at codon 59, or the MEK1 mutation at codon 387, 56, 121, 124, or 129 correlates with a decreased response or resistance to treatment of said melanoma with a BRAF inhibitor, e.g. the inhibitor of Compound A. Likewise, it is asserted that the combined occurrence of any of the above described deletions or mutations in V600 mutated BRAF melanoma correlates with a decreased response or resistance to treatment of said melanoma with a BRAF inhibitor, e.g. the inhibitor of Compound A. Further, it is asserted that the above described deletions or mutations in V600 mutated BRAF melanoma, occurring alone or in any combination, correlates with an increased likelihood of efficacious treatment of said melanoma with the combination of Compound B and Compound C as described herein.

Compound B or pharmaceutically acceptable salts thereof, for use in combination with Compound C, may be prepared as described above.

Compound C, and pharmaceutically acceptable salts and solvates thereof, are disclosed and claimed, along with pharmaceutically acceptable salts thereof, as being useful as an inhibitor of PI3K activity, particularly in treatment of cancer, in International Publication Number WO2008/144463, the entire disclosure of which is hereby incorporated by reference. Compound C may also act as an m-TOR inhibitor and is known to have m-TOR inhibitory activity. Compound B is the compound exemplified and may be prepared in accordance with example 345 of the WO'463 publication.

The combined administration of Compound B and Compound C is disclosed, along with suitable formulations and dosing, in PCT/US2010/050495, filed Sep. 28, 2010, which teachings are incorporated by reference and which may be relied on as instructive as to the various possible methods of administering the combination.

When used in combination, Compound B and/or Compound C may form a solvate which is understood to be a complex of variable stoichiometry formed by a solute (in this invention, Compound B or a salt thereof and/or Compound C or a salt thereof) and a solvent. Such solvents for the purpose of the invention may not interfere with the biological activity of the solute. Examples of suitable solvents include, but are not limited to, water, methanol, dimethyl sulfoxide, ethanol and acetic acid. Suitably the solvent used is a pharmaceutically acceptable solvent. Examples of suitable pharmaceutically acceptable solvents include, without limitation, water, dimethyl sulfoxide, ethanol and acetic acid. Suitably the solvent used is water.

Suitably, Compound C is provided in the form of its free base. Alternatively, the pharmaceutically acceptable salts of the Compounds of the invention are readily prepared by those of skill in the art and in accordance with this disclosure.

Suitably, the amount of Compound B (amount of free or unsalted and unsolvated compound per dose) administered as part of the combination of Compound B and Compound C is an amount selected from about 0.125 mg to about 10 mg; for instance, the amount may be about 5 mg.

Suitably, the amount of Compound C (amount of free or unsalted and unsolvated compound per dose) administered as part of the combination of Compound B and Compound C will be an amount selected from about 0.25 mg to about 75 mg; suitably, the amount will be selected from about 6 mg to about 12 mg; suitably, the amount will be about 10 mg.

### B RAF+PI3K

In further embodiments, the combination of a BRaf inhibitor and a PI3K inhibitor may be used. Suitably Compound A, as described above, and Compound C, as described above, may be used.

Surprisingly, V600 mutant melanoma that exhibits NRAS Q61 and/or A146 mutation or a MEK1 mutation is treatable with a combination of a BRAF inhibitor and a PI3K inhibitor such that cell growth inhibition exceeds the additive effect of the individual agents.

By way of example, V600 mutant melanoma that exhibits NRAS Q61 and/or A146 mutation has been shown to respond with renewed cell growth inhibition upon treatment with the combination of the PI3K inhibitor of formula (III)

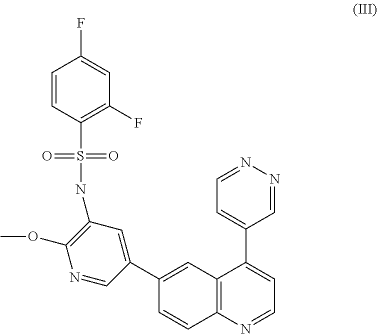 [SMILES: COc1ncc(-c2ccc3nccc(-c4ccnnc4)c3c2)cc1NS(=O)(=O)c1ccc(F)cc1F]

(hereinafter referred to as “Compound C”) or pharmaceutically acceptable salts or solvates thereof and the BRaf inhibitor Compound A or pharmaceutically acceptable salts or solvates thereof.

As shown in the Examples section below, the combination of PI3K inhibitor Compound C or pharmaceutically acceptable salts thereof and the BRaf inhibitor Compound A or pharmaceutically acceptable salts or solvates thereof inhibit cellular proliferation of tumor cells that have previously been exposed to BRaf inhibitor Compound A and which have developed resistance to BRaf inhibitor Compound A, as demonstrated by the examples and as correlated to the exhibition of NRAS Q61 and/or A146 mutation. The combination provides superior inhibition of the NRAS mutated cells over either of the single agents.

According to an embodiment of the invention, there is a method of treating a human suffering from V600 mutant melanoma comprising the steps of administering a first BRaf inhibitor, determining if the melanoma tumor tissue of said human has an NRAS Q61 and/or A146 mutation, and administering a combination of a PI3K inhibitor and BRAF inhibitor if said NRAS Q61 and/or A146 mutation is present.

According to an embodiment of the invention, there is a method of treating a human suffering from V600 mutant melanoma comprising the steps of administering BRaf inhibitor Compound A or pharmaceutically acceptable salt thereof, determining if the melanoma tumor tissue of said human has an NRAS Q61 and/or A146 mutation, and administering a combination of a PI3K inhibitor and BRaf inhibitor if said NRAS Q61 and/or A146 mutation is present. Advantageously, the BRaf inhibitor is Compound A or a pharmaceutically acceptable salt or solvate thereof.

According to an embodiment of the invention, there is a method of treating a human suffering from V600 mutant melanoma comprising the steps of administering BRaf inhibitor Compound A or pharmaceutically acceptable salt thereof, determining if the melanoma tumor tissue of said human has an NRAS Q61 and/or A146 mutation, and administering a combination of PI3K inhibitor Compound C or pharmaceutically acceptable salt thereof and BRaf inhibitor of Compound A or a pharmaceutically acceptable salt thereof.

Surprisingly, V600 mutant melanoma that exhibits a MEK1 K59 deletion, a MEK1 P387 mutation, a MEK1 Q56 mutation, a MEK1 C121 mutation, a MEK1 P124 mutation, or a MEK1 F129 mutation is treatable with a combination of PI3K/mTOR inhibitor and BRaf inhibitor such that cell growth inhibition exceeds the additive effect of the individual agents.

By way of example, V600 mutant melanoma that exhibits a MEK1 K59 deletion, a MEK1 P387 mutation, a MEK1 Q56 mutation, a MEK1 C121 mutation, a MEK1 P124 mutation, or a MEK1 F129 mutation has been shown to respond with renewed cell growth inhibition upon treatment with the combination of the PI3K inhibitor of Compound C or pharmaceutically acceptable salts or solvates thereof and the BRaf inhibitor Compound A or pharmaceutically acceptable salts thereof.

As shown in the Examples section below, the combination of PI3K inhibitor Compound C or pharmaceutically acceptable salts thereof and the BRaf inhibitor Compound A or pharmaceutically acceptable salts or solvates thereof inhibit cellular proliferation of tumor cells that have previously been exposed to BRaf inhibitor Compound A and which have developed resistance to BRaf inhibitor Compound A, as demonstrated by the examples and as correlated to the exhibition of a MEK1 K59 deletion, a MEK1 P387 mutation, a MEK1 Q56 mutation, a MEK1 C121 mutation, a MEK1 P124 mutation, or a MEK1 F129 mutation. The combination provides superior inhibition of the MEK1 mutated cells over either of the single agents.

According to an embodiment of the invention, there is a method of treating a human suffering from V600 mutant melanoma comprising the steps of administering a first BRaf inhibitor, determining if the melanoma tumor tissue of said human has a MEK1 K59 deletion, a MEK1 P387 mutation, a MEK1 Q56 mutation, a MEK1 C121 mutation, a MEK1 P124 mutation, or a MEK1 F129 mutation, and administering a combination of a PI3K inhibitor and BRaf inhibitor if said MEK1 K59 deletion, MEK1 P387 mutation, MEK1 Q56 mutation, MEK1 C121 mutation, MEK1 P124 mutation, or MEK1 F129 mutation is present.

According to an embodiment of the invention, there is a method of treating a human suffering from V600 mutant melanoma comprising the steps of administering BRaf inhibitor Compound A or pharmaceutically acceptable salt thereof, determining if the melanoma tumor tissue of said human has a MEK1 K59 deletion, a MEK1 P387 mutation, a MEK1 Q56 mutation, a MEK1 C121 mutation, a MEK1 P124 mutation, or a MEK1 F129 mutation, and administering a combination of a PI3K inhibitor and BRaf inhibitor if said MEK1 K59 deletion, MEK1 P387 mutation, MEK1 Q56 mutation, MEK1 C121 mutation, MEK1 P124 mutation, or MEK1 F129 mutation is present. Advantageously, the BRaf inhibitor is Compound A or a pharmaceutically acceptable salt thereof.

According to an embodiment of the invention, there is a method of treating a human suffering from V600 mutant melanoma comprising the steps of administering BRaf inhibitor Compound A or pharmaceutically acceptable salt thereof, determining if the melanoma tumor tissue of said human has a MEK1 K59 deletion, a MEK1 P387 mutation, a MEK1 Q56 mutation, a MEK1 C121 mutation, a MEK1 P124 mutation, or a MEK1 F129 mutation, and administering a combination of PI3K inhibitor Compound C or pharmaceutically acceptable salt thereof and BRaf inhibitor of Compound A or a pharmaceutically acceptable salt thereof.

According to an embodiment of the invention, there is a method of treating a human suffering from V600 mutant melanoma comprising the steps of administering BRaf N-{3-[5-(2-Amino-4-pyrimidinyl)-2-(1,1-dimethylethyl)-1,3-thiazol-4-yl]-2-fluorophenyl}-2,6-difluorobenzenesulfonamide methanesulfonate, determining if the melanoma tumor tissue of said human has an NRAS Q61 and/or A146 mutation, and administering a combination of 2,4-difluoro-N-{2-(methyloxy)-5-[4-(4-pyridazinyl)-6-quinolinyl]-3-pyridinyl}benzenesulfonamide and N-{3-[5-(2-Amino-4-pyrimidinyl)-2-(1,1-dimethylethyl)-1,3-thiazol-4-yl]-2-fluorophenyl}-2,6-difluorobenzenesulfonamide methanesulfonate.

According to an embodiment of the invention, there is a method of treating a human suffering from V600 mutant melanoma comprising the steps of administering N-{3-[5-(2-Amino-4-pyrimidinyl)-2-(1,1-dimethylethyl)-1,3-thiazol-4-yl]-2-fluorophenyl}-2,6-difluorobenzenesulfonamide methanesulfonate, determining if the melanoma tumor tissue of said human has a MEK1 K59 deletion, a MEK1 P387 mutation, a MEK1 Q56 mutation, a MEK1 C121 mutation, a MEK1 P124 mutation, or a MEK1 F129 mutation, and administering a combination of 2,4-difluoro-N-{2-(methyloxy)-5-[4-(4-pyridazinyl)-6-quinolinyl]-3-pyridinyl}benzenesulfonamide and N-{3-[5-(2-Amino-4-pyrimidinyl)-2-(1,1-dimethylethyl)-1,3-thiazol-4-yl]-2-fluorophenyl}-2,6-difluorobenzenesulfonamide methanesulfonate.

For the avoidance of confusion, it is asserted that any of the above described deletions or mutations in V600 mutated BRAF melanoma, i.e. any one of: the NRAS mutation at codon 61, the NRAS mutation at codon 146, the MEK1 deletion at codon 59, or the MEK1 mutation at codon 387, 56, 121, 124, or 129 correlates with a decreased response or resistance to treatment of said melanoma with a BRAF inhibitor, e.g. the inhibitor of Compound A. Likewise, it is asserted that the combined occurrence of any of the above described deletions or mutations in V600 mutated BRAF melanoma correlates with a decreased response or resistance to treatment of said melanoma with a BRAF inhibitor, e.g. the inhibitor of Compound A. Further, it is asserted that the above described deletions or mutations in V600 mutated BRAF melanoma, occurring alone or in any combination, correlates with an increased likelihood of efficacious treatment of said melanoma with the combination of Compound A and Compound C as described herein.

Compound A or pharmaceutically acceptable salts thereof, for use in combination with Compound C, may be prepared as described above.

Compound C, and pharmaceutically acceptable salts and solvates thereof, are disclosed and claimed, along with pharmaceutically acceptable salts thereof, as being useful as an inhibitor of PI3K activity, particularly in treatment of cancer, in International Publication Number WO2008/144463, the entire disclosure of which is hereby incorporated by reference. Compound C may also act as an m-TOR inhibitor and is known to have m-TOR inhibitory activity.

The combined administration of Compound A and Compound C is disclosed, along with suitable formulations and dosing, in PCT/US2010/052242 (published as International Publication number WO2011/046894), each of which is incorporated by reference and which may be relied on as instructive as to the various possible methods of administering the combination.

When used in combination, Compound A and/or Compound C may form a solvate which is understood to be a complex of variable stoichiometry formed by a solute (in this invention, Compound A or a salt thereof and/or Compound C or a salt thereof) and a solvent. Such solvents for the purpose of the invention may not interfere with the biological activity of the solute. Examples of suitable solvents include, but are not limited to, water, methanol, dimethyl sulfoxide, ethanol and acetic acid. Suitably the solvent used is a pharmaceutically acceptable solvent. Examples of suitable pharmaceutically acceptable solvents include, without limitation, water, dimethyl sulfoxide, ethanol and acetic acid. Suitably the solvent used is water.

Suitably, Compound C is provided in the form of its free base. Alternatively, the pharmaceutically acceptable salts of the Compounds of the invention are readily prepared by those of skill in the art and in accordance with this disclosure.

Suitably, the amount of Compound A (amount of free or unsalted and unsolvated compound per dose) administered as part of the combination of Compound A and Compound C is an amount selected from about 0.125 mg to about 10 mg; for instance, the amount may be about 5 mg.

Suitably, the amount of Compound C (amount of free or unsalted and unsolvated compound per dose) administered as part of the combination of Compound A and Compound C will be an amount selected from about 0.25 mg to about 75 mg; suitably, the amount will be selected from about 6 mg to about 12 mg; suitably, the amount will be about 10 mg.

### Further Combinations

Additional embodiments employ treatment with other BRaf, MEK, e.g. MEK1, and PI3K inhibitors. For example, in one embodiment the BRaf inhibitors is PLX4032. In a further embodiment, treatment comprises administering at least PLX4032 and Compound B, or PLX4032 and Compound C. In another embodiment, treatment comprises administering PLX4032 and Compound A and Compound C.

Additional embodiments employ at least one of the following MEK1 inhibitors: AZD6244, AE6201 from Eisai Co. Ltd., AS 703026 from Merck KGaA, R7420 from Roche, R7167 from Roche and Chugai Pharmaceutical Co. Ltd and TAK-733 from Takeda Pharmaceutical Co. Ltd., RDEA119 from Ardea Bioscience Inc. and Bayer and WX-555 from UCB Group. In a preferred embodiment, the MEK1 inhibitor is an allosteric MEK1 inhibitor. Additional embodiments include MEK1 inhibitors recited in Fremin et al., Journal of Hematology & Oncology 2010, 3:8, which is incorporated by reference in part to reference the chemical structures and names of the MEK1 inhibitors therein. In a further embodiment, treatment comprises administering a MEK1 inhibitor recited in this paragraph or described in a reference incorporated herein and Compound A, or a MEK1 inhibitor recited in this paragraph or described in a reference incorporated herein and Compound C.

In another embodiment, at least one of the following PI3K inhibitors is employed: CAL101, PX-866, BKM120, XL147, GDC0941, PX-866, CAL101, BEZ235, BGT226, XL765, SF1126. The structures of these compounds as shown in are incorporated by reference herein. In a further embodiment, treatment comprises administering at least one of the following combinations: a PI3K inhibitor recited in this paragraph and Compound A, a PI3K inhibitor recited in this paragraph and Compound B, a PI3K inhibitor, PLX4032 and Compound B.

Typically, any anti-neoplastic agent that has activity versus a susceptible tumor being treated may be co-administered in the treatment of cancer in the present invention. Examples of such agents can be found in Cancer Principles and Practice of Oncology by V. T. Devita and S. Hellman (editors), 6^{th} edition (Feb. 15, 2001), Lippincott Williams & Wilkins Publishers. Typical anti-neoplastic agents useful for combination with the BRaf, MEK, and PI3K inhibitors discussed above include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; receptor tyrosine kinase inhibitors; serine-threonine kinase inhibitors; non-receptor tyrosine kinase inhibitors; angiogenesis inhibitors, immunotherapeutic agents; proapoptotic agents; and cell cycle signalling inhibitors.

The present invention also provides methods for treating V600 mutant melanoma comprising administering MEK inhibitor of Compound B or pharmaceutically acceptable salt thereof in combination with either the BRaf inhibitor of Compound A or a pharmaceutically acceptable salt thereof and/or the PI3K inhibitor of Compound C or pharmaceutically acceptable salts thereof, in addition to another anti-neoplastic agent. Examples of a further active ingredient or ingredients (anti-neoplastic agent) for use in such combination are as follows:

Anti-microtubule or anti-mitotic agents, such as diterpenoids and vinca alkaloids (such as vinblastine, vincristine, and vinorelbine); diterpenoids, such as paclitaxel (TAXOL®) and its analog docetaxel; platinum coordination complexes, such as cisplatin and carboplatin; alkylating agents, such as cyclophosphamide, melphalan, and chlorambucil; alkyl sulfonates such as busulfan; nitrosoureas such as carmustine; and triazenes such as dacarbazine.

Additional anti-neoplastic agents that may be used in combination with the invention include antibiotic anti-neoplastics, such as actinomycins such as dactinomycin, anthracyclins such as daunorubicin and doxorubicin; and bleomycins; topoisomerase inhibitors, such as epipodophyllotoxins, such as etoposide and teniposide.

Additional anti-neoplastic agents that may be used in combination with the invention include antimetabolite neoplastic agents, such as fluorouracil (5-fluoro-2,4-(1H,3H) pyrimidinedione, 5-fluoro deoxyuridine (floxuridine) and 5-fluorodeoxyuridine monophosphate), methotrexate, cytarabine, mercaptopurine (PURINETHOL®), thioguanine (TABLOID®), and gemcitabine (GEMZAR®).

Additional anti-neoplastic agents that may be used in combination with the invention include camptothecins, including, camptothecin and camptothecin derivatives available or under development as Topoisomerase I inhibitors, such as irinotecan, topotecan, and the various optical forms of 7-(4-methylpiperazino-methylene)-10,11-ethylenedioxy-20-camptothecin; Innotecan HCl (CAMPTOSAR®); Irinotecan; and Topotecan HCl (HYCAMTIN®).

Further anti-neoplastic agents that may be used in combination with the invention include rituximab (RITUXAN® and MABTHERA®); ofatumumab (ARZERRA®); mTOR inhibitors include but are not limited to rapamycin and rapalogs, RAD001 or everolimus (Afinitor), CCI-779 or temsirolimus, AP23573, AZD8055, WYE-354, WYE-600, WYE-687 and Pp121; bexarotene (Targretin®); and sorafenib (Nexavar®).

The invented combination may be used in combination with hormones useful in treating cancer. Examples of hormones and hormonal analogues useful in cancer treatment include, but are not limited to, adrenocorticosteroids such as prednisone and prednisolone; aminoglutethimide and other aromatase inhibitors such as anastrozole, letrazole, vorazole, and exemestane; progestrins such as megestrol acetate; estrogens, androgens, and anti-androgens such as flutamide, nilutamide, bicalutamide, cyproterone acetate and 5α-reductases such as finasteride and dutasteride; anti-estrogens such as tamoxifen, toremifene, raloxifene, droloxifene, iodoxyfene, as well as selective estrogen receptor modulators (SERMS) such those described in U.S. Pat. Nos. 5,681,835, 5,877,219, and 6,207,716; and gonadotropin-releasing hormone (GnRH) and analogues thereof which stimulate the release of leutinizing hormone (LH) and/or follicle stimulating hormone (FSH) for the treatment prostatic carcinoma, for instance, LHRH agonists and antagagonists such as goserelin acetate and luprolide.

The invented combination may be used in further combination with signal transduction pathway inhibitors, such as inhibitors of receptor tyrosine kinases, non-receptor tyrosine kinases, SH2/SH3 domain blockers, serine/threonine kinases, phosphotidyl inositol-3 kinases, myo-inositol signaling, and Ras oncogenes, including growth factor receptors include, for example, epidermal growth factor receptor (EGFr), platelet derived growth factor receptor (PDGFr), erbB2, erbB4, ret, vascular endothelial growth factor receptor (VEGFr), tyrosine kinase with immunoglobulin-like and epidermal growth factor homology domains (TIE-2), insulin growth factor-I (IGFI) receptor, macrophage colony stimulating factor (cfms), BTK, ckit, cmet, fibroblast growth factor (FGF) receptors, Trk receptors (TrkA, TrkB, and TrkC), ephrin (eph) receptors, and the RET protooncogene. An exemplary signal transduction pathway inhibitor is lapatinib (Tykerb/Tyverb®), a dual EGFR/ErbB2 inhibitor.

Tyrosine kinases, which are not growth factor receptor kinases are termed non-receptor tyrosine kinases. Non-receptor tyrosine kinases useful in the present invention, which are targets or potential targets of anti-cancer drugs, include cSrc, Lck, Fyn, Yes, Jak, cAbl, FAK (Focal adhesion kinase), Brutons tyrosine kinase, and Bcr-Abl. Such non-receptor kinases and agents which inhibit non-receptor tyrosine kinase function are described in Sinh, S. and Corey, S. J., (1999) Journal of Hematotherapy and Stem Cell Research 8 (5): 465-80; and Bolen, J. B., Brugge, J. S., (1997) Annual review of Immunology. 15: 371-404.

SH2/SH3 domain blockers are agents that disrupt SH2 or SH3 domain binding in a variety of enzymes or adaptor proteins including, P13-K p85 subunit, Src family kinases, adaptor molecules (Shc, Crk, Nck, Grb2) and Ras-GAP. SH2/SH3 domains as targets for anti-cancer drugs are discussed in Smithgall, T. E. (1995), Journal of Pharmacological and Toxicological Methods. 34(3) 125-32.

Inhibitors of Serine/Threonine Kinases including MAP kinase cascade blockers which include blockers of Raf kinases (rafk), Mitogen or Extracellular Regulated Kinase (MEKs), and Extracellular Regulated Kinases (ERKs); and Protein kinase C family member blockers including blockers of PKCs (alpha, beta, gamma, epsilon, mu, lambda, iota, zeta). IkB kinase family (IKKa, IKKb), PKB family kinases, akt kinase family members, and TGF beta receptor kinases. Such Serine/Threonine kinases and inhibitors thereof are described in Yamamoto, T., Taya, S., Kaibuchi, K., (1999), Journal of Biochemistry. 126 (5) 799-803; Brodt, P, Samani, A., and Navab, R. (2000), Biochemical Pharmacology, 60. 1101-1107; Massague, J., Weis-Garcia, F. (1996) Cancer Surveys. 27:41-64; Philip, P. A., and Harris, A. L. (1995), Cancer Treatment and Research. 78: 3-27, Lackey, K. et al Bioorganic and Medicinal Chemistry Letters, (10), 2000, 223-226; U.S. Pat. No. 6,268,391; and Martinez-Iacaci, L., et al, Int. J. Cancer (2000), 88(1), 44-52.

Inhibitors of Phosphotidyl inositol-3 Kinase family members including blockers of P13-kinase, ATM, DNA-PK, and Ku are also useful in the present invention. Such kinases are discussed in Abraham, R. T. (1996), Current Opinion in Immunology. 8 (3) 412-8; Canman, C. E., Lim, D. S. (1998), Oncogene 17 (25) 3301-3308; Jackson, S. P. (1997), International Journal of Biochemistry and Cell Biology. 29 (7):935-8; and Zhong, H. et al, Cancer res, (2000) 60(6), 1541-1545.

Also useful in the present invention are Myo-inositol signaling inhibitors such as phospholipase C blockers and Myoinositol analogues. Such signal inhibitors are described in Powis, G., and Kozikowski A., (1994) New Molecular Targets for Cancer Chemotherapy ed., Paul Workman and David Kerr, CRC press 1994, London.

Another group of signal transduction pathway inhibitors are inhibitors of Ras Oncogene. Such inhibitors include inhibitors of farnesyltransferase, geranyl-geranyl transferase, and CAAX proteases as well as anti-sense oligonucleotides, ribozymes and immunotherapy. Such inhibitors have been shown to block ras activation in cells containing wild type mutant ras, thereby acting as antiproliferation agents. Ras oncogene inhibition is discussed in Scharovsky, O. G., Rozados, V. R., Gervasoni, S. I. Matar, P. (2000), Journal of Biomedical Science. 7(4) 292-8; Ashby, M. N. (1998), Current Opinion in Lipidology. 9 (2) 99-102; and BioChim. Biophys. Acta, (19899) 1423(3):19-30.

As mentioned above, antibody antagonists to receptor kinase ligand binding may also serve as signal transduction inhibitors. This group of signal transduction pathway inhibitors includes the use of humanized antibodies to the extracellular ligand binding domain of receptor tyrosine kinases. For example Imclone C225 EGFR specific antibody (see Green, M. C. et al, Monoclonal Antibody Therapy for Solid Tumors, Cancer Treat. Rev., (2000), 26(4), 269-286); Herceptin® erbB2 antibody (see Tyrosine Kinase Signalling in Breast cancer:erbB Family Receptor Tyrosine Kinases, Breast cancer Res., 2000, 2(3), 176-183); and 2CB VEGFR2 specific antibody (see Brekken, R. A. et al, Selective Inhibition of VEGFR2 Activity by a monoclonal Anti-VEGF antibody blocks tumor growth in mice, Cancer Res. (2000) 60, 5117-5124).

Anti-angiogenic agents including non-receptor anti-angiogenesis inhibitors may also be useful. Anti-angiogenic agents such as those which inhibit the effects of vascular endothelial growth factor (for example the anti-vascular endothelial cell growth factor antibody bevacizumab [Avastin™]) and compounds that work by other mechanisms (for example linomide, inhibitors of integrin αvβ3 function, endostatin and angiostatin);

Agents used in immunotherapeutic regimens may also be useful in combination with the compounds of invention. Immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies

Agents used in proapoptotic regimens (e.g., bcl-2 antisense oligonucleotides) may also be used in the combination of the present invention.

Cell cycle signalling inhibitors, including CDK2, CDK4, and CDK6 and inhibitors for the same are described in, for instance, Rosania et al, Exp. Opin. Ther. Patents (2000) 10(2):215-230.

### Method of Treating

Therapeutically effective amounts of the combinations of the invention are administered to a human. Typically, the therapeutically effective amount of the administered agents of the present invention will depend upon a number of factors including, for example, the age and weight of the subject, the precise condition requiring treatment, the severity of the condition, the nature of the formulation, and the route of administration. Ultimately, the therapeutically effective amount will be at the discretion of the attendant physician. Guidance with regard to dosing is provided herein with regard to the administration of Compound A, and with regard to the described combinations of Compounds.

As used herein, the term “treatment” or “treating” in the context of therapeutic methods, refers to alleviating the specified condition, eliminating or reducing the symptoms of the condition, slowing or eliminating the progression, invasion, or metastatic spread of the condition and preventing or delaying the reoccurrence of the condition in a previously afflicted subject. The present invention further provides use of the compounds of the invention for the preparation of a medicament for the treatment of several conditions in a mammal (e.g., human) in need thereof.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

The Compounds may be employed in combination in accordance with the invention by administration simultaneously in a unitary pharmaceutical composition including both compounds. Alternatively, the combination may be administered separately in separate pharmaceutical compositions, each including one of the Compounds in a sequential manner wherein, for example, one of the Compounds is administered first and the other second. Such sequential administration may be close in time (e.g. simultaneously) or remote in time. Furthermore, it does not matter if the compounds are administered in the same dosage form, e.g. one compound may be administered topically and the other compound may be administered orally. Suitably, both compounds are administered orally. Thus in one embodiment of the invention, one or more doses of the first Compound are administered simultaneously or separately with one or more doses of the other Compound.

According to the invention, one of the Compounds may be administered as a loading dose, where the term “loading dose” as used herein will be understood to mean a single dose or short duration regimen of one or the other Compound having a dosage higher than the maintenance dose administered to the subject to, for example, rapidly increase the blood concentration level of the drug.

The term “maintenance dose” as used herein will be understood to mean a dose that is serially administered (for example; at least twice), and which is intended to either slowly raise blood concentration levels of the compound to a therapeutically effective level, or to maintain such a therapeutically effective level. The maintenance dose is generally administered once per day and the daily dose of the maintenance dose is lower than the total daily dose of the loading dose.

Suitably the combinations of this invention are administered within a “specified period”. By the term “specified period” and derivatives thereof, as used herein is meant the interval of time between the administration of one of Compounds and the other of the Compounds. Unless otherwise defined, the specified period can include simultaneous administration. When both Compounds of the invention are administered once a day the specified period refers to administration of the first and second Compounds during a single day. When one or both Compounds are administered more than once a day, the specified period is calculated based on the first administration of each compound on a specific day. All administrations of a Compound that are subsequent to the first during a specific day are not considered when calculating the specific period. Suitably, if the Compounds are administered within a “specified period” and not administered simultaneously, they are both administered within about 24 hours of each other. As used herein, the administration of both Compounds in less than about 45 minutes apart is considered simultaneous administration. Suitably, both compounds will be administered within a specified period for at least one day and continued until treatment is no longer needed.

Alternatively, during the course of treatment, the Compounds may be administered within a first period of time within a second period of time, such as for from 1 to 4 days over a 7 day period, with either a single Compound or neither of the Compounds administered during the second period of time.

The Compounds may be administered sequentially. By the term “sequential administration”, and derivates thereof, as used herein is meant that one of the Compounds is administered for two or more consecutive days and the other of the Compounds is subsequently administered for two or more consecutive days. Also, contemplated herein is a drug holiday utilized between the sequential administration of the Compounds. As used herein, a drug holiday is a period of days, for instance from 1 to 14 days, after the sequential administration of one of the Compounds and before the administration of the other of the Compounds where neither of the Compounds is administered.

Regarding sequential administration, suitably, one of the Compounds is administered for from 1 to 30 consecutive days, followed by an optional drug holiday, followed by administration of the other of the Compounds for from 1 to 30 consecutive days.

### Further Definitions

As used herein the term “agent” is understood to mean a substance that produces a desired effect in a tissue, system, animal, mammal, human, or other subject. Accordingly, the term “anti-neoplastic agent” is understood to mean a substance producing an anti-neoplastic effect in a tissue, system, animal, mammal, human, or other subject. It is also to be understood that an “agent” may be a single compound or a combination or composition of two or more compounds.

Typically, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts derived from a nitrogen on a substituent in a compound of the present invention. Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium and valerate. Other salts, which are not pharmaceutically acceptable, may be useful in the preparation of compounds of this invention and these form a further aspect of the invention. Salts may be readily prepared by a person skilled in the art.

While it is possible that, for use in therapy, the compounds used in accordance with the invention, may be administered as the raw chemical, it is possible to present the active ingredient as a pharmaceutical composition. Accordingly, the invention further provides pharmaceutical compositions, which include one or more combination of compounds, and one or more pharmaceutically acceptable carriers, diluents, or excipients. The carrier(s), diluent(s) or excipient(s) must be acceptable in the sense of being compatible with the other ingredients of the formulation, capable of pharmaceutical formulation, and not deleterious to the recipient thereof. In accordance with another aspect of the invention there is also provided a process for the preparation of a pharmaceutical composition including admixing the compounds, with one or more pharmaceutically acceptable carriers, diluents or excipients. Such elements of the pharmaceutical compositions utilized may be presented in separate pharmaceutical combinations or formulated together in one pharmaceutical composition. Accordingly, the invention further provides a method of treating or use of combination of pharmaceutical compositions one of which includes one of Compounds A, B, or C, and one or more pharmaceutically acceptable carriers, diluents, or excipients and a pharmaceutical composition containing another of Compounds A, B, or C, and one or more pharmaceutically acceptable carriers, diluents, or excipients.

Pharmaceutical compositions may be presented in unit dose forms containing a predetermined amount of active ingredient per unit dose. As is known to those skilled in the art, the amount of active ingredient per dose will depend on the condition being treated, the route of administration and the age, weight and condition of the patient. Preferred unit dosage compositions are those containing a daily dose or sub-dose, or an appropriate fraction thereof, of an active ingredient. Furthermore, such pharmaceutical compositions may be prepared by any of the methods well known in the pharmacy art.

### EXPERIMENTALS

### Methods

### Chemicals and Reagents

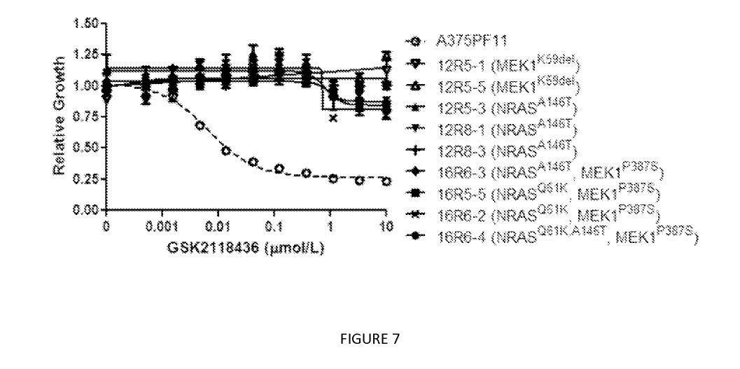

Phospho-AKT (T308), phospho-AKT (S473), AKT, ERK, phospho-S6P (S235/236), S6P, cyclin D1, phospho-MEK1/2 (S217/S221), MEK1/2, MEK1, MEK2, and cleaved PARP antibodies were purchased from Cell Signaling. Primary antibodies to GAPDH, RAF-1, BRAF, and ARAF were purchased from Santa Cruz Biotechnology. Diphospho-ERK1/2 (T202/Y204) antibodies were purchased from Sigma and p27kip1 antibodies were purchased from Epitomics. Anti-Mouse IRDYE® 800CW antibody was purchased from LI-COR Biosciences. Anti-rabbit ALEXA FLUOR® 680 antibodies were purchased from Invitrogen. Compound A, Compound B, Compound C, and PLX4032 (20) were synthesized by GlaxoSmithKline Research Labs and the structures are shown in FIG. 7.

### Cell Lines

A clonal isolate, hereinafter referred to as “A375 F11”, was obtained from the A375 cell line (obtained from ATCC), based on increased sensitivity to a BRAF inhibitor. A375 F11 cells were exposed to increasing concentrations of Compound A and maintained in a final concentration of 1.26 μM or 1.6 μM. 12R5-3, 12R8-1, 12R8-3, 16R5-5, 16R6-2, 16R6-3 and 16R6-4 were derived as single cell clones from mixed populations of these A375 F11 cells selected to grow in the presence of Compound A. All cells were grown in RPMI 1640 medium containing 10% Fetal Bovine Serum (FBS).

The YUSIT1 melanoma cells were obtained from Yale Dermatology Cell Culture Facility. A total of 13 YUSIT1 Compound A resistant clones were obtained based on increased sensitivity to the BRAF inhibitor Compound A. Specifically, YUSIT1 cells were exposed to increasing concentrations of Compound A and maintained in a final concentration of 0.1 umole/L of Compound A. Single cell clones, e.g., YUSIT1-B5, YUSIT1-B11, YUSIT1-B31 clones were isolated by limited dilution from populations that could proliferate in the presence of Compound A. Cells were grown in the same media as A375 F11 cells.

### PCR and Sequence Analysis

gDNA isolation was prepared using the Promega Maxwell 16 Cell DNA Purification Kit (Promega, Madison Wis.). All gDNA PCR primers were ordered from IDT (Integrated DNA Technologies Inc, Coralville, Iowa). PCR primers were tailed with M13 universal sequencing primer sequences. PCR reactions were carried out using Roche FastPCR DNA polymerase (ROCHE, Indianapolis, Ind.). DNA was amplified for 35 cycles at 95° C. for 20 seconds, 60° C. for 30 seconds and 72° C. for 45 seconds followed by 7 minutes extension at 72° C. All gDNA primers have been tested (QC) on Promega Human Genomic DNA (Promega, Madison Wis.), before using on cell line samples. PCR products were purified using Agencourt AmPure (Agencourt Bioscience Corporation, Beverly, Mass.). Direct sequencing of purified PCR products was performed with AB v3.1 BigDye-terminator cycle sequencing kit (Applied Biosystems, Foster City, Calif.) and sequencing reactions were purified using Agencourt CleanSeq (Agencourt Bioscience Corporation, Beverly, Mass.). The sequencing reactions were analyzed using a Genetic Analyzer 3730XL (Applied Biosystems, Foster City, Calif.). All sequence results data were assembled and analyzed using Codon Code Aligner (CodonCode Corporation, Dedham, Mass.). The primer sequences are:

| | | SEQ ID NO: |
|---|---|---|
| NRAS.NM_002524.3 | | |
| Exon02 forward | tgtaaaacgacggccagtTGAGGCCGATATTAATCCGGTG | 1 |
| Exon02 reverse | caggaaacagctatgaccTCGCTACTATGGCCTGTGTTTCTCA | 2 |
| Exon03 forward | tgtaaaacgacggccagtCCAGATAGGCAGAAATGGGCTTG | 3 |
| Exon03 reverse | caggaaacagctatgaccGGTTCCAAGTCATTCCCAGTAGCA | 4 |
| Exon03b forward | tgtaaaacgacggccagtGAAATGGGCTTGAATAGTTA | 5 |
| Exon03b reverse | caggaaacagctatgaccCTTCCCTAGTGTGGTAACCTC | 6 |
| Exon04 forward | tgtaaaacgacggccagtATGAGCCACTGTACCCAGCCTAAT | 7 |
| Exon04 reverse | caggaaacagctatgaccTTTGGGTTTAAACAAGCGTTAAGAAA | 8 |
| Exon04b forward | tgtaaaacgacggccagtGTGCTGAGATTGCAGGCATG | 9 |
| Exon04b reverse | caggaaacagctatgaccATGAATATGGATCACATCTC | 10 |
| Exon05 forward | tgtaaaacgacggccagtCAACACCAGCCCGTTTATGGC | 11 |
| Exon05 reverse | caggaaacagctatgaccTGCAGAAGAGGATAGGCAGAAACTCA | 12 |
| BRAF.NM_004333.4 | | |
| Exon01 forward | TGTAAAACGACGGCCAGTCCGGCTCTCGGTTATAAGATG | 13 |
| Exon01 reverse | CAGGAAACAGCTATGACCAAATAAACACCAGCCAGCCG | 14 |
| Exon02 forward | TGTAAAACGACGGCCAGTGGAAGGCTGCTCACCAAACCA | 15 |
| Exon02 reverse | CAGGAAACAGCTATGACCGGCAAAGCTAATTCTCTCTTCCCA | 16 |
| Exon03 forward | TGTAAAACGACGGCCAGTTGTCAGGACAAAGTCCGGATTGA | 17 |
| Exon03 reverse | CAGGAAACAGCTATGACCGACATGACTGTGGTTCAAGTTTGGC | 18 |
| Exon04 forward | TGTAAAACGACGGCCAGTTGATGGATATACTGCGTTGGTGGG | 19 |
| Exon04 reverse | CAGGAAACAGCTATGACCTGGCCTACAGTATTTCTTCAGGCTAAC | 20 |
| Exon04b forward | TGTAAAACGACGGCCAGTGTTGTATCTGACCTAGTAAC | 21 |
| Exon04b reverse | CAGGAAACAGCTATGACCCCTACAGTATTTCTTCAGGC | 22 |
| Exon05 forward | TGTAAAACGACGGCCAGTTAATAACCAAGAAAGGCTTG | 23 |
| Exon05 reverse | CAGGAAACAGCTATGACCATTCATTCATTAAAATCTAA | 24 |
| Exon06 forward | TGTAAAACGACGGCCAGTGCTTGAAATCAGTTGCCAGCCT | 25 |
| Exon06 reverse | CAGGAAACAGCTATGACCTGGGAGAGAAATACTGTCCATTCCAC | 26 |
| Exon07 forward | TGTAAAACGACGGCCAGTAAATTTCTCTTTGCTGTATGCCAGTTT | 27 |
| Exon07 reverse | CAGGAAACAGCTATGACCCCCAAGAGCAGAAGTCAAACCA | 28 |
| Exon08 forward | TGTAAAACGACGGCCAGTCCTGGAGGGCCTACTTGAGCA | 29 |
| Exon08 reverse | CAGGAAACAGCTATGACCAATGTCATCAGAGAGAAACCAGAAGC | 30 |
| Exon09 forward | TGTAAAACGACGGCCAGTTGGCCTGAAATGGACATCAACA | 31 |
| Exon09 reverse | CAGGAAACAGCTATGACCTGTGTCTGTTACTTGAAAGAGGCAAG | 32 |
| Exon10 forward | TGTAAAACGACGGCCAGTTTTGGCCCATCCTTTCCAA | 33 |
| Exon10 reverse | CAGGAAACAGCTATGACCTGAAATATCTGAATTCTGGACCAGCC | 34 |
| Exon11 forward | TGTAAAACGACGGCCAGTTCTCTTCCTGTATCCCTCTCAGGCA | 35 |
| Exon11 reverse | CAGGAAACAGCTATGACCGGAGTCCCGACTGCTGTGAA | 36 |
| Exon12 forward | TGTAAAACGACGGCCAGTCCATGGAACAAACAAGGTTGGC | 37 |
| Exon12 reverse | CAGGAAACAGCTATGACCGCTACCACTGGGAACCAGGAGC | 38 |
| Exon13 forward | TGTAAAACGACGGCCAGTAAGCAACCTCTAGTTGCCTGTCAGA | 39 |
| Exon13 reverse | CAGGAAACAGCTATGACCTCTGAAGGCTGCACTAGACAGAGACA | 40 |
| Exon14 forward | TGTAAAACGACGGCCAGTGGCTTGACTGGAGTGAAAGGTTTG | 41 |
| Exon14 reverse | CAGGAAACAGCTATGACCCAGGCTGTGGTATCCTGCTCTCC | 42 |
| Exon15 forward | TGTAAAACGACGGCCAGTCATAATGCTTGCTCTGATAG | 43 |
| Exon15 reverse | CAGGAAACAGCTATGACCTAACTCAGCAGCATCTCAGG | 44 |
| Exon16 forward | TGTAAAACGACGGCCAGTCAGGAATGGGAAGTGGGAACTGA | 45 |
| Exon16 reverse | CAGGAAACAGCTATGACCTCCTTCACGCTTACCCAGGAGTT | 46 |
| Exon17 forward | TGTAAAACGACGGCCAGTTTTGTGGGTTTCCCACCATCT | 47 |
| Exon17 reverse | CAGGAAACAGCTATGACCTGCTCAACCCTCATGAAGCCA | 48 |
| Exon18 forward | TGTAAAACGACGGCCAGTGGTGCTTTCTTGTAAAGTGTGATGG | 49 |
| Exon18 reverse | CAGGAAACAGCTATGACCCAAACCCGGAACAGAAAGTAAAGC | 50 |
| Exon18b forward | TGTAAAACGACGGCCAGTGTGTGATGGGACTCTTAAAG | 51 |
| Exon18b reverse | CAGGAAACAGCTATGACCGTTGCTACTCTCCTGAACTC | 52 |
| Exon18c forward | TGTAAAACGACGGCCAGTGTAGATTCTCGCCTCTATTG | 53 |
| Exon18c reverse | CAGGAAACAGCTATGACCGTTGCTACTCTCCTGAACTC | 54 |
| MEK1.NM_002755.3 | | |
| Exon 01 forward | tgtaaaacgacggccagtAGCGGGAGGAAGCGAGAGGT | 55 |
| Exon 01 reverse | caggaaacagctatgaccCCCAGACGTACGTGACAAACCA | 56 |
| Exon 02 forward | tgtaaaacgacggccagtCGGGTGGCTGGAGTGAAGTG | 57 |
| Exon 02 reverse | caggaaacagctatgaccCAGTCTTCCTTCTACCCTGGTCCC | 58 |
| Exon 03 forward | tgtaaaacgacggccagtCCGAGGCACAGCTGGAACAG | 59 |
| Exon 03 reverse | caggaaacagctatgaccGGAGGCCTGCCATCTTGTGA | 60 |
| Exon 04 forward | tgtaaaacgacggccagtCATAGGACAAGGTATGGGAGTGGG | 61 |
| Exon 04 reverse | caggaaacagctatgaccTCCAGACAGGGAAGAGTCTAGTGAGAA | 62 |
| Exon 05 forward | tgtaaaacgacggccagtGGAGGAAGGCAAATTTGTGATGA | 63 |
| Exon 05 reverse | caggaaacagctatgaccAGCTGGCCAGTCCTGGCTTT | 64 |
| Exon 06 forward | tgtaaaacgacggccagtCCAAGGGCTGCCTCTGATGG | 65 |
| Exon 06 reverse | caggaaacagctatgaccGCCACACTCTGGAGCTGGACA | 66 |
| Exon 07 forward | tgtaaaacgacggccagtCAGCCAGGGCACAGCTGATT | 67 |
| Exon 07 reverse | caggaaacagctatgaccTAGCCTGGCATCCAGGGACA | 68 |
| Exon 08 forward | tgtaaaacgacggccagtTGTGGCTGTTTAATGTTTATTGTCCA | 69 |
| Exon 08 reverse | caggaaacagctatgaccTGAACCTGTGACAGCGATGTGTT | 70 |
| Exon 09 forward | tgtaaaacgacggccagtTACGTTCCCATGCCGCACTC | 71 |
| Exon 09 reverse | caggaaacagctatgaccTCAGACGGGAGGGTAAAGGGC | 72 |
| Exon 10 forward | tgtaaaacgacggccagtGGATGAATCAAGCCCAGGCA | 73 |
| Exon 10 reverse | caggaaacagctatgaccTGCCACCAAACGAGACAGCA | 74 |
| Exon 11 forward | tgtaaaacgacggccagtCACCACGTCCTCTCGTTTCCTT | 75 |
| Exon 11 reverse | caggaaacagctatgaccAGGATCTCACAAGGCTCCCTCC | 76 |
| MEK2.NM_030662.3 | | |
| Exon 1 forward | tgtaaaacgacggccagtCTATGGGCCCCGGCTAGAGG | 77 |
| Exon 1 reverse | caggaaacagctatgaccGCCTCGTGCACTCCTCGCGAA | 78 |
| Exon 2 forward | tgtaaaacgacggccagtCCCAGAAGCTGGGCCCTTTC | 79 |
| Exon 2 reverse | caggaaacagctatgaccCCGCCCTGGATTCTGGTCAT | 80 |
| Exon 3 forward | tgtaaaacgacggccagtTGGCATCGACTGCCTTGAGAA | 81 |
| Exon 3 reverse | caggaaacagctatgaccTGACCACTGTTGGGAACGCC | 82 |
| Exon 4 forward | tgtaaaacgacggccagtAGGTCCTGGCGTCCCACATC | 83 |
| Exon 4 reverse | caggaaacagctatgaccCGTGGGTGGAGTTGGGCTC | 84 |
| Exon 4b forward | tgtaaaacgacggccagtACGCATCCGTGGCTTCCCG | 85 |
| Exon 4b reverse | caggaaacagctatgaccGGCAGATGGTGACCTCAGGC | 86 |
| Exon 5 and 6 | tgtaaaacgacggccagtGTTGGGAAAGCCTCGTGGGA | 87 |
| forward | | |
| Exon 5 and 6 | caggaaacagctatgaccGCGAGCTCCTCACAGCCTGA | 88 |
| reverse | | |
| Exon 5b forward | tgtaaaacgacggccagtCAGCTCTGACTGCTCAGCTC | 89 |
| Exon 5b reverse | caggaaacagctatgaccGCCATGGAGTCGATGAGCTG | 90 |
| Exon 6b forward | tgtaaaacgacggccagtCAGATCATGCACCGAGGTAAG | 91 |
| Exon 6b reverse | caggaaacagctatgaccGACAGCTGCGCAGGAGACATG | 92 |
| Exon 7 forward | tgtaaaacgacggccagtGACCACAGTCGGCACCATCC | 93 |
| Exon 7 reverse | caggaaacagctatgaccCTGCGGTCAACACCAGCTCC | 94 |
| Exon 8 forward | tgtaaaacgacggccagtGGGAAGGAGCTGGGCTTGTG | 95 |
| Exon 8 reverse | caggaaacagctatgaccCGCCTCCAATTTAGGCTGGC | 96 |
| Exon 8b forward | tgtaaaacgacggccagtCAGTGCCTCTGCACACCCGC | 97 |
| Exon 8b reverse | caggaaacagctatgaccCTCTTGCTCTGGTCAGAGAG | 98 |
| Exon 9 forward | tgtaaaacgacggccagtACAGGGCCTGCAATCCTGCT | 99 |
| Exon 9 reverse | caggaaacagctatgaccAGTTGGCATGTGGTGTCGGC | 100 |
| Exon 10 forward | tgtaaaacgacggccagtCCAGAGCTGTTCCCACGCAC | 101 |
| Exon 10 reverse | caggaaacagctatgaccCCCTGGCACCAAAGTGCAGA | 102 |
| Exon 11 forward | tgtaaaacgacggccagtGCTCAGGGATGTCCTCTCCG | 103 |
| Exon 11 reverse | caggaaacagctatgaccGGAAGGGAGGGTCTCCTCTGTTT | 104 |
| Exon 11b forward | tgtaaaacgacggccagtGTCCTACTGTGACTCCAGG | 105 |
| Exon 11b reverse | caggaaacagctatgaccGCCAGCCTGTCCTCAGCTG | 106 |

Each primer is tailed with a M13 universal primer. The tail is lowercase (used for sequencing). The primers denoted “b” are an alternate primer used to amplify the designated exon.
Cloning and FLAG-Tagging of Human Neuroblastoma RAS Viral Oncogene Homolog (NRAS) cDNA

Primers used to PCR amplify human NRAS-WT were designed based on UTR flanking regions of the published human 5′ and 3′ UTR sequence (NM_{—}002524.3). The full length ORF was amplified using the primers NRASfornest (5′-ATGACTGAGTACAAACTGGTG 3′) (SEQ ID NO: 107) and NRASrevNEST (5′-TTACATCACCACACATGGCAATC-3′) (SEQ ID NO: 108). The resulting ORF was TOPO-cloned into pCR BLUNT vector (Invitrogen, Carlsbad, Calif.) and double strand sequence confirmed. This plasmid was used as template for the NRAS-WT FLAG-tag and mutagenesis reactions. 5′ FLAG-tag was incorporated using the primers NRASflagtag (5′ caccATGGATTACAAGGATGACGACGATAAGGGA atg act gagtacaaactggtggtgg-3′) (SEQ ID NO: 109) and NRASrevNEST (5′TTACATCACCACACATGGCAATC-3′) (SEQ ID NO: 110). Human bone marrow cDNA (Marathon Ready, Clontech, Mountainview, Calif.) was used as the template and Phusion polymerase (New England BioLabs, Ipswich, Mass.) was used for all PCR reactions. The primary and nested reactions were carried out according to vendor instructions using the following parameters: 29 cycles at 98° C. for 10s, 55° C. for 30 s and 72° C. for 15 s per kb, followed by a final 10 minute extension at 72° C., in a MJ Research PTC200 thermocycler. The FLAG-tag incorporation annealed at 59° C. The resulting ORF was TOPO-cloned into pcDNA3.1D (Invitrogen) and double strand sequence confirmed.

### Mutagenesis NRAS: Q61K, Q61R, and A146T

Full length ORF of human NRAS (NM_{—}002524.3) was prepared as above and used as template for mutagenesis reactions and FLAG-tag reactions. Pfu Ultra polymerase (Stratagene, Santa Clara, Calif.) was used for PCR amplification. The reaction was performed for 16 cycles at 95° C. for 30 s, 60° C. for 60s and 72° C. for 14 min, hold at 4° C. After mutagenesis was completed, DpNI enzyme was added to each reaction and a 37° C. incubation was completed before transformation. Clones were sequenced, to verify the desired mutation. Following sequence analysis, a plasmid prep was prepared and the 5′ FLAG-tag was incorporated as above. The resulting ORF was TOPO-cloned into pcDNA3.1D (Invitrogen) and double strand sequence confirmed.

### Mutagenesis Primers:

Q61Kfor
(SEQ ID NO: 111)
(5′-GTTTGTTGGACATACTGGATAC AGC TGG AAA AGAAGAGTA
CAGTGCCATGAGAGACCAAT-3′)
Q61Krev
(SEQ ID NO: 112)
(5′-TTGGTCTCTCATGGCACTGTACTCTTCTTTTCCAGCTGTATCC
AGTA TGTCCAACAAAC-3′)
Q61Rfor
(SEQ ID NO: 113)
(5-GTTTGTTGGACATACTGGATACAGCTGGACGAGAAGAGTACAGTG
CCATGAG AGACCAAT-′3)
Q61Rrev
(SEQ ID NO: 114)
(5′-ATTGGTCTCTCATGGCACTGTACTCTTCTCGTCCAGCTGTATC
CAGTATGTCCAACAAAC-′)
A146Tfor
(SEQ ID NO: 115)
(5′-GTTACGGGATTCCATTCATTGAAACCTCAACCAAGACCAGACA
GGGTGTTGAAGATGCTT-′3)
A146Trev
(SEQ ID NO: 116)
(5′-AAGCATCTTCAACACCCTGTCTGGTCTTGGTTGAGGTTTCAAT
GAATGGAATCCCGTAAC-3′)
NRASflagTAG
(SEQ ID NO: 117)
(5′-caccATGGATTACAAGGATGACGACGATAAGGGAatgactgag
tacaaactggtggtgg-3′)

### BacMAM Plasmid Construction.

The coding regions of human MEK1 with EcoRI and BamHI was PCR amplified and the PCR products were digested with EcoRI and BamHI. The PCR products were cloned into pFNcmvNA vector (1) digested with EcoRI and BamHI. The resultant pFNcmvNA-human MEK1 plasmid was mutated via QuickChange Lighting site-directed mutagenesis (Agilent Technologies) with the primers used at the following positions:

| | | SEQ ID |
|---|---|---|
| Primer | Sequence | NO: |
| MEK1wt-fwd | GAATTCGGTACCATGCCCAAGAAGAAGCCGACGCCCAT | 118 |
| rev | GTTAACGGATCCTTAGACGCCAGCAGCATGGGTTGGTGTGC | 119 |
| MEK1Q56P-fwd | CGCCTTGAGGCCTTTCTTACCccgAAGCAGAAGGTGGGAGAACTG | 120 |
| rev | CAGTTCTCCCACCTTCTGCTTcggGGTAAGAAAGGCCTCAAGGCG | 121 |
| MEK1Het59delk-fwd | GAGGCCTTTCTTACCCAGAAGcaggtgGGAGAACTGAAGGATGACGAC | 122 |
| rev | GTCGTCATCCTTCAGTTCTCCcacctgCTTCTGGGTAAGAAAGGCCTC | 123 |
| MEK1P387S-fwd | GGCCTTAACCAGCCCAGCACAtcaACCCATGCTGCTGGCGTCtaa | 124 |
| -rev | TtaGACGCCAGCAGCATGGGTtgaTGTGCTGGGCTGGTTAAGGCC | 125 |
| MEK1C121S-fwd | AGGGAGCTGCAGGTTCTGCATGAGtccAACTCTCCGTACATCGTGGGCTTC | 126 |
| rev | GAAGCCCACGATGTACGGAGAGTTggaCTCATGCAGAACCTGCAGCTCCCT | 127 |

### BacMam Generation

BacMam viruses were generated essentially as described (1), which is based on the Bac-to-Bac system that of LifeTechnologies (www.invitrogen.com). Briefly pFNcmvNA-human MEK1 and pFNcmvNA-human MEK2 and their mutant derivatives were transformed into DH10Bac cells. The resulting recombinant bacmid DNA was used to transfect Sf9 cells. After 3-4 days, P0 batches of BacMams were harvested, which were used to infect Sf9 cells to generate P1 batch BacMams.

### RAS Activation Assay:

Amount of activated RAS was determined using RAS Activation ELISA (Millipore). Briefly, 100 micrograms of total protein lysed in magnesium lysis buffer was added to RAF-1-RBD coated glutathione plate. Captured RAS was detected by chemiluminescence using anti-Ras antibody on a plate reader. Activated RAS was reported as chemiluminescent signal in arbitrary units (AU) after subtraction of no protein added control.

### Cloning Assay:

Cells were plated at 5K per well in a 6-well dish (BD Falcon) in RPMI medium containing 10% FBS. The following day, cells were treated with indicated compound in RPMI medium containing 10% FBS. Compound treatment were replaced once during the 14 day assay. After 14 days, media was removed and cells were stained in a solution of 0.5% Methylene Blue in 50% ethanol for at least 30 minutes. Stain was removed and plates were rinsed by submission in a distilled water and air-dried. Complete images of stained 6-well plates were captured as JPEG files using flatbed scanner (HP).

### Cell Growth Inhibition Assay and Combination Data Analysis.

All cells were cultured without compound, e.g. Compound A, for a minimum of 72 hours prior to cell plating. Cells were assayed in a 96-well tissue culture plate (NUNC 136102) of RPMI medium containing 10% FBS for all cells at 1,000 cells per well. Approximately 24 hours after plating, cells were exposed to ten, three-fold serial dilutions of compound or the combination of the two agents at a constant molar to molar ratio, e.g. 10:1 Compound A to Compound B, in RPMI media containing 10% FBS. Cells were incubated in the presence of compounds for 3 days. ATP levels were determined by adding Cell Titer Glo® (Promega) according to the manufacturer's protocol. Briefly, Cell Titer Glo® was added each plate, incubated for 30 minutes then luminescent signal was read on the SpectraMax L plate reader with a 0.5 sec integration time. Inhibition of cell growth was estimated after treatment with compound or combination of compounds for three days and comparing the Cell Titer Glo® signal to cells treated with vehicle (DMSO). Cell growth was calculated relative to vehicle (DMSO) treated control wells. Concentration of compound that inhibits 50% of control cell growth (IC₅₀) was interpolated when y=50% of the vehicle control using nonlinear regression with the equation, y=(A+(B−A)/(1+(C/x)̂D)), where A is the minimum response (yₘᵢₙ), B is the maximum response (yₘₐₓ), C is the inflection point of the curve (EC₅₀) and D is the Hill coefficient.

Combination effects on potency were evaluated using Combination Index (CI) which was calculated with the back-interpolated IC₅₀ values and the mutually non-exclusive equation derived by Chou and Talalay (1): CI=Da/IC₅₀(a)+Db/IC₅₀(b)+(Da×Db)/(IC₅₀(a)×IC₅₀(b))

where IC₅₀(a) is the IC₅₀ of Compound A; IC₅₀(b) is the IC₅₀ for Compound B; Da is the concentration of Compound A in combination with Compound B that inhibited 50% of cell growth; and Db is the concentration of Compound B in combination with Compound A that inhibited 50% of cell growth. In general, a CI value <0.9, between 0.9 and 1.1, or >1.1 indicates synergy, additivity and antagonism, respectively. In general, the smaller the CI number, the greater is the strength of synergy.

The combination effects on the response scale were quantified by Excess Over Highest Single Agent (EOHSA) based on the concept of nonlinear blending as described in detail by Peterson and Novick (2007) and Peterson (2010) [(2;3) [Peterson and Novick, 2007; Peterson, 2010]. EOHSA values are defined as increases in improvement (here, in ‘percentage points’ (ppts) difference) produced by the combination over the best single agent at its component dose level for the combination. For single agent and combination treatments, cells were exposed to compounds at a fixed-dose-ratio, and dose response curves were fit to the experimental data and analyzed using regression models. At specified total dose levels of IC₅₀ along the dose response curve, the dose combination (corresponding to IC₅₀) was determined for making EOHSA statistical inferences. More specifically, for a combination drug experiment involving drug 1 at dose d1 and drug 2 at dose d2, (i.e., total dose equals d1+d2) is said to have a positive EOHSA if the mean response at the combination is better than the mean response to drug 1 at dose d1 or drug 2 at dose d2.

### Transfection of A375 Cells

A375 cells were transfected with indicated NRAS constructs with lipofectamine 2000 (Invitrogen). Stable single cell transfectants were selected with 0.1 μM Compound A and isolated with a cloning cylinder. Colonies were observed after transfection of the vector, NRAS wild-type and NRAS mutant plasmids into A375 when selected with G418. Transfected clones were maintained in RPMI 1640 medium containing 10% FBS and 0.1 μM Compound A and plated without Compound A for proliferation and western blot experiments.

Small Hairpin RNA (shRNA) Mediated Knockdown of NRAS

A375 were transduced with lentivirus expressing shRNAs targeting NRAS (TRCN0000300442) or non-targeting control (SHC002V) from Sigma Aldrich (St. Louis). Cells with stable integration of the shRNA lentivirus were selected with puromycin and single cell clones isolated with cloning cylinders. Stably integrated clones were maintained in puromycin for subsequent experiments.

siRNA Mediated Knockdown

Single oligo siRNAs (Dharmacon) for MAP2K1 (D-003571-01, D-003571-04 and D-003571-05), MAP2K2 (D-003573-01 and D-003573-02) and non-targeting controls (D-001210-02 and D-001210-05) were reverse transfected twice into indicated cells 48 hours apart using RNAiMAX (Invitrogen). One day after second transfection, the cells were plated for proliferation and protein extraction. Protein was extracted 24 hours after plating. Proteins were subjected to western blot analysis as described above. Relative cell growth after 72 hours was determined by Cell Titer Glo®.

### BacMAM Transduction of A375 Cells

BacMAM constructs were generated using MEK1 cDNA and mutations in MEK1 were introduced using site directed mutagenesis. BacMAM virus particles were prepared as described in the supplemental methods. A375 cells were transduced with the indicated BacMAM virus. Forty-eight hours post transduction, cells were plated for subsequent proliferation and western blot analysis as described above.

### Compound B MEK1 Model Construction

Crystal structure 3EQG (23) was prepared for model building using the protein preparation wizard in Maestro (Schrodinger, LLC). Hydrogens were added, double bonds were assigned, some waters were deleted, H-bonds were assigned and finally, the structure was energy minimized using Impref and the OPLS2005 force field. A heavy atom convergence threshold of 0.15 angstroms was used for this step. The small molecule Compound B was roughly aligned by hand with the allosteric inhibitor in the structure and then this other inhibitor was removed. The Compound B Mek1 complex was energy minimized by holding the protein fixed, using Macromodel in Maestro and the OPLS2005 force field. The acetylated anilino moiety in Compound B appeared to require further refinement and was subjected to a conformational search using Macromodel. Finally, the ligand and non-backbone atoms up to 7 angstroms away were energy minimized using Macromodel. Visual inspection of the model indicated only one potentially questionable interaction between the acetyl NH of Compound B and a nearby arginine. However, because this portion of the inhibitor is under the flexible P-loop and is also solvent exposed, the model was judged fit for illustrative purposes.

### DNA Analysis

SNP analysis on Affymetrix Genome Wide Human SNP Array 6.0 chips verified that the clones that acquired resistance to Compound A were derived from A375 as they shared greater than 95% similarity as determined by identical by state and identical by decent calculations in Array Studio (Omicsoft).

### Affymetrix Expression Analyses

16R6-4 was selected for comparison with A375 after compound treatment with Compound A and Compound B alone and in combination with each other for 24 hours. Two or three independent samples were used for each clone or each treatment of clone. RNA and cDNA were prepared and microarray analyses were performed at Response Genetics Inc. (Los Angeles, Calif.) using Affymetrix HG-U133Plus2 array according to the standard Affymetrix protocol. RNA microarray signals were normalized by MASS. QC and data analysis were performed using Array Studio (Omicsoft). Samples with a MAD score <−5 were excluded from further analysis. Differentially expressed genes were identified by applying one-way ANOVA with p value <0.05, and >2-fold change. Probe sets that, which met the cut off criteria, but had a mean intensity under 100 were excluded.

### Results

### A375 F11 Melanoma Cells

Ten lines derived from A375 F11 melanoma cells that acquired resistance to Compound A were isolated after exposure of A375 F11 cells to increasing concentrations of Compound A. Genomic DNA was sequenced for BRAF, NRAS and MAP2K1 (MEK1). As shown in Table 1, all clones retained the homozygous mutation of BRAF V600E observed in the parental A375 F11 cells. In addition to BRAF V600E, the Compound A resistant melanoma cells also have heterozygous mutations of MEK1 K59 deletion (12R5-1 and 12R5-5), NRAS Q61R or A146T (12R9, 12R5-5, 12R5-3, 12R8-1) or both NRAS Q61K and/or A146T and MEK1 P387S (16R6-3, 15R5-5, 16R6-2 and 16R6-4). Specifically, 12R5-1 and 12R5-5 cells have deletions of AAG at 175-177 nucleotides (Het 175-177delAAG) on MAP2K1 gene resulting in lysine 59 deletion (Het 59delK) in MEK1; 12R5-3, 12R8-1 and 12R8-3 lines have heterozygous mutation of NRAS G436A resulting in a change of alanine at amino acid 146 to threonine (A146T); 16R6-3 cells have heterozygous mutations of both NRAS G436A (A146T) and MAP2K1 C1159T which resulting in a change of proline at amino acid 387 to serine (P387P/S); 12R9 line has a heterozygous mutation of NRAS A182G resulting in a change of glutamine at amino acid 61 to arginine (Q61R); 16R5-5 and 16R6-2 lines have heterozygous mutations of NRAS C181A resulting in a change of glutamine at amino acid 61 to lysine (Q61K) and MAP2K1 C1159/T (P387S); and 16R6-4 cells have heterozygous mutations of NRAS C181A (Q61K), NRAS G436A (A146T), and MAP2K1 C1159/T (P387S). Sequencing confirmed the retention of the homozygous BRAF^{V600E} mutation in these clones with no additional BRAF mutation. In addition, no mutations in KRAS, HRAS, ARAF, CRAF or PTEN were identified. All clones retained a synonymous MEK2 polymorphism at I220 from the A375 parental line. The above data indicate that mutations of MEK1 K59 deletion, NRAS Q61K/R and/or A146T, or both NRAS Q61K and/or A146T and MEK1 P387S mutation are associated with resistance to a BRAF inhibitor, Compound A in BRAF V600 mutation melanoma.

TABLE 1
Genomic DNA Sequence Analysis of BRAF and NRAS

| Cell | | | | | | |
|---|---|---|---|---|---|---|
| Line | BRAF | BRAF | NRAS | NRAS | MEK1 | MAP2K1 |
| F11 | V600E | T1799A | WT | WT | WT | WT |
| 12R5-1 | V600E | T1799A | WT | WT | Het | Het |
| | | | | | 59delK | 175-177 |
| | | | | | | delAAG |
| 12R5-5 | V600E | T1799A | WT | WT | Het | Het |
| | | | | | 59delK | 175-177 |
| | | | | | | delAAG |
| 12R5-3 | V600E | T1799A | A146A/T | G436G/A | WT | WT |
| 12R8-1 | V600E | T1799A | A146A/T | G436G/A | WT | WT |
| 12R8-3 | V600E* | T1799A | A146A/T | G436G/A | WT | WT |
| 16R6-3 | V600E | T1799A | A146A/T | G436G/A | P387P/ | C1159C/T |
| | | | | | S | |
| 12R9 | V600E | T1799A | Q61Q/R | A182A/G | ND | ND |
| 16R5-5 | V600E | T1799A | Q61Q/K | C181C/A | P387P/ | C1159C/T |
| | | | | | S | |
| 16R6-2 | V600E | T1799A | Q61Q/K | C181C/A | P387P/ | C1159C/T |
| | | | | | S | |
| 16R6-4 | V600E | T1799A | Q61Q/K, | C181C/A, | P387P/ | C1159C/T |
| | | | A146A/T | G436G/A | S | |

*only exon 15 of BRAF sequenced
ND, not determined

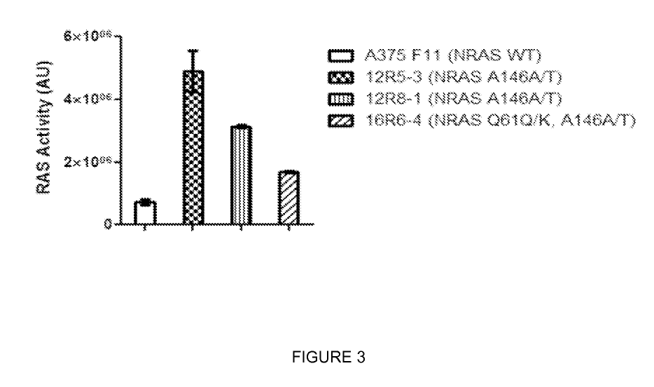

The detection of NRAS Q61 and/or A146T mutations in Compound A acquired resistant cells harboring BRAF V600E mutation was striking and unexpected. Further, as shown in FIG. 3, the resistant cells 16R6-4, 12R5-3 and 12R8-1 with BRAF V600E and NRAS mutations at Q61K and/or A146T increased RAS GTPase activity in comparison to melanoma cells without these NRAS mutations (A375 F11 cells). The increased RAS GTPase activity is assumed to be due to the NRAS mutations. For instance, NRAS Q61 mutation has been reported to increase NRAS activity by locking the Ras protein in the GTP-bound state with a subsequent continuous activation of its downstream effectors (Taparowsky et al, cell, 34:581-586, 1983).

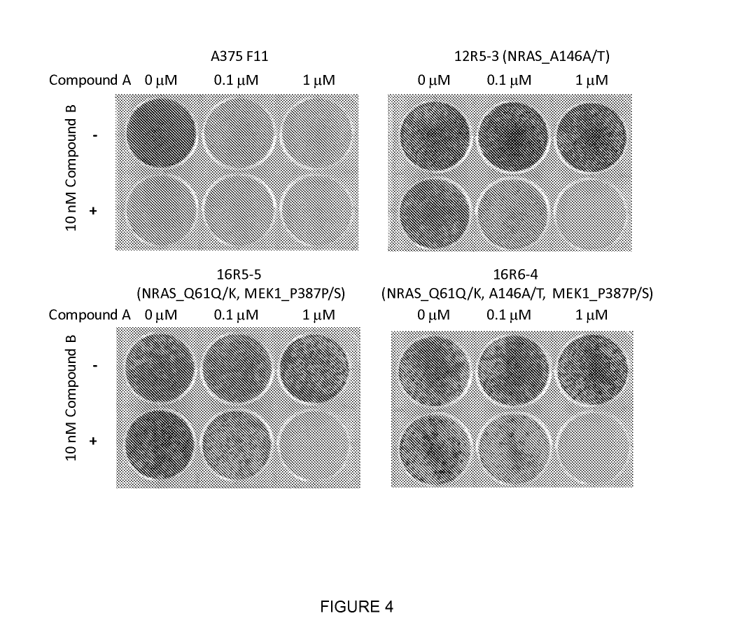

The effect of Compound A, Compound B or their combination on cell growth inhibition was determined in the A375 F11 clones that acquired resistance to Compound A (Table 2 and FIG. 4). A375 F11 BRAF V600E mutation cells with wild type NRAS and MEK1 were highly sensitive to cell growth inhibition by Compound A or Compound B as single agents and in combination. In contrast, BRAF V600E mutated lines, 12R5-1 and 12R5-5 with MEK1 59delK mutation, 12R5-3, 12R8-1 and 12R8-3 with NRAS A146T mutation, 16R6-3 line with both NRAS A146T and MEK1 P387S, 12R9 with NRAS Q61R, 16R5-5 and 16R6-2 with NRAS Q61/K and MEK1 P387S, and 16R6-4 with NRAS Q61/K, NRAS A146T and MEK1 P387S mutations were resistant to BRAF inhibitor Compound A (IC50>10 μM), and showed reduced sensitivity to MEK inhibitor Compound B. However these BRAF V600E mutated lines with either NRAS or MEK1 or both NRAS and MEK1 mutations were sensitive to the combination of Compound A and Compound B with IC50s ranging from 0.178-0.392 μM for Compound A and 0.018 to 0.039 μM for Compound B. The combination of Compound A and Compound B in a 10:1 molar ratio showed enhanced cell growth inhibition as demonstrated by EOSHA.

TABLE 2
Cell Growth Inhibition by Compound A, Compound B and their combination in
Compound A Acquired Resistant Clones

| | | | | | | | Combination |
|---|---|---|---|---|---|---|---|
| | | | IC₅₀ values in micromolar (mean ± std) | | | | Effects at |
| | | | | | 10:1 Molar Ratio | | IC₅₀ |
| Cell | Mutation Status | | Single Agent Effect | | Combination | | EOHSA |
| Line | NRAS | MEK1 | Compound A | Compound B | Compound A | Compound B | (ppt) |
| F11 | WT | WT | 0.024 ± 0.017 | 0.002 ± 0.002 | 0.006 ± 0.005 | 0.0006 ± 0.0005 | 19.2 ± 1.3 |
| 12R5-1 | WT | HET | >10 | >1 | 0.392 ± 0.149 | 0.039 ± 0.015 | 53.0 ± 13.7 |
| | | 59delK | | | | | |
| 12R5-5 | WT | HET | >10 | >1 | 0.383 ± 0101 | 0.038 ± 0.010 | 46.0 ± 11.7 |
| | | 59delK | | | | | |
| 12R5-3 | A146A/T | WT | >10 | 0.078 ± 0.029 | 0.252 ± 0.070 | 0.025 ± 0.007 | 27.9 ± 5.3 |
| 12R8-1 | A146A/T | WT | >10 | 0.081 ± 0.031 | 0.266 ± 0.136 | 0.027 ± 0.014 | 29.2 ± 8.8 |
| 12R8-3 | A146A/T | WT | >10 | 0.059 ± 0.037 | 0.178 ± 0.182 | 0.018 ± 0.018 | 27.4 ± 0.7 |
| 16R6-3 | A146A/T | P387P/S | >10 | 0.162 ± 0.067 | 0.280 ± 0.123 | 0.028 ± 0.012 | 35.7 ± 9.4 |
| 16R5-5 | Q61Q/K | P387P/S | >10 | 0.129 ± 0.054 | 0.268 ± 0.072 | 0.027 ± 0.007 | 36.3 ± 3.4 |
| 16R6-2 | Q61Q/K | P387P/S | >10 | 0.099 ± 0.058 | 0.242 ± 0.057 | 0.024 ± 0.006 | 28.3 ± 7.9 |
| 16R6-4 | Q61Q/K, | P387P/S | >10 | >1 | 0.223 ± 0.144 | 0.022 ± 0.014 | 41.8 ± 9.7 |
| | A146A/T | | | | | | |

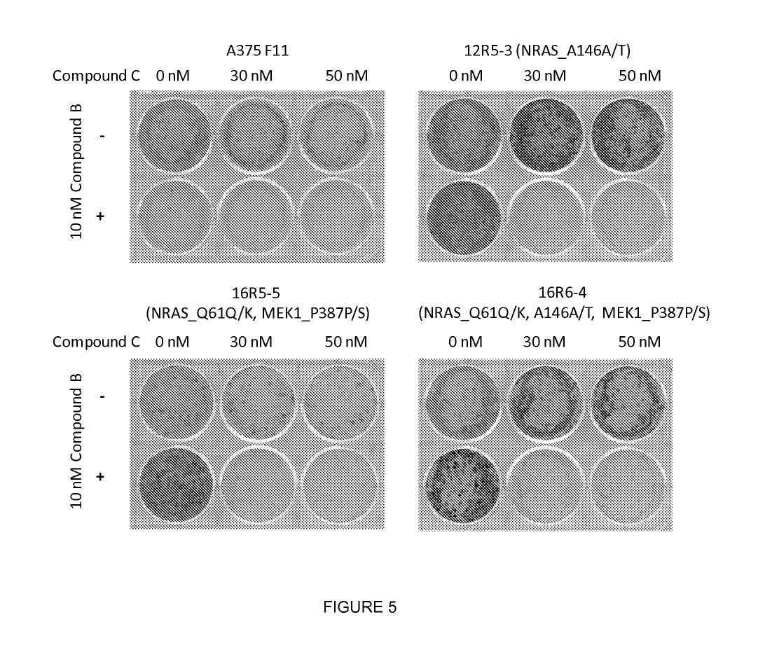

Similarly, as shown in Table 3 and FIG. 5, BRAF V600E mutant melanoma cells with resistance to the BRAF inhibitor of Compound A and co-mutations in MEK1 59delK; NRAS A146; or NRAS Q61K and/or A146 and MEK1 P387; respectively, were sensitive to the combination of the MEK inhibitor of Compound B and the PI3K inhibitor of Compound C. The combination of Compounds B and C had IC₅₀, ranging from 0.013-0.043 μM. Equal molar combination of Compound B and Compound C was synergistic with CI values between 0.25 and 0.67 and/or enhanced cell growth inhibition with EOHSA values ≧10 ppt in all the melanoma cells with the co-mutations as shown.

TABLE 3
Cell Growth Inhibition by Compound B, Compound C and their combination in
Compound A Acquired Resistant Clones

| | | | IC₅₀ values in micromolar (mean ± std) | | | Combination Effects at | |
|---|---|---|---|---|---|---|---|
| | Mutational | | | | 1:1 Molar | IC₅₀ | |
| CELL | Analysis | | Single Agent | | Ratio | | EOHSA |
| LINE | NRAS | MEK1 | Compound B | Compound C | Combination | CI | (ppt) |
| F11 | WT | WT | 0.006 ± 0.004 | 0.098 ± 0.058 | 0.004 ± 0.002 | 0.86 ± 0.28 | 4.5 ± 5.8 |
| 12R5-1 | WT | HET | >1 | 0.155 ± 0.130 | 0.037 ± 0.007 | N/A | 24.8 ± 21.0 |
| | | 59delK | | | | | |
| 12R5-5 | WT | HET | 0.315 ± 0.212 | 0.087 ± 0.001 | 0.020 ± 0.003 | 0.32 ± 0.02 | 28.6 ± 2.2 |
| | | 59delK | | | | | |
| 12R5-3 | A146A/T | WT | 0.132 ± 0.054 | 0.159 ± 0.140 | 0.027 ± 0.007 | 0.53 ± 0.09 | 20.6 ± 12.1 |
| 12R8-1 | A146A/T | WT | 0.056 ± 0.039 | 0.145 ± 0.047 | 0.021 ± 0.004 | 0.67 ± 0.17 | 13.2 ± 12.2 |
| 12R8- | A146A/T | WT | 0.084 | 0.375 | 0.059 | 0.97 | 10.3 |
| 3* | | | | | | | |
| 16R6-3 | A146A/T | P387P/S | 0.137 ± 0.077 | 0.162 ± 0.099 | 0.020 ± 0.006 | 0.34 ± 0.11 | 29.6 ± 6.1 |
| 12R9* | Q61Q/R | ND | 0.024 | 0.057 | 0.008 | 0.47 | 22.7 |
| 16R5- | Q61Q/K | P387P/S | 0.097 ± 0.000 | 0.091 ± 0.036 | 0.020 ± 0.003 | 0.49 ± 0.03 | 22.9 ± 9.7 |
| 5* | | | | | | | |
| 16R6-2 | Q61Q/K | P387P/S | 0.339 ± 0.366 | 0.111 ± 0.087 | 0.013 ± 0.002 | 0.26 ± 0.16 | 31.8 ± 4.0 |
| 16R6-4 | Q61Q/K, | P387P/S | 0.475 ± 0.587 | 0.213 ± 0.169 | 0.043 ± 0.036 | 0.37 ± 0.07 | 29.5 ± 2.6 |
| | A146A/T | | | | | | |

*n = 1
ND, not determined
Cell growth assays demonstrated that the A375 clones were resistant to Compound A (IC₅₀ > 10 μmol/L) (FIG. 7). In addition, the A375 clones were resistant to another BRAF inhibitor, PLX4032 (IC₅₀ > 10 μmol/L, data not shown), and displayed greater than 10-fold

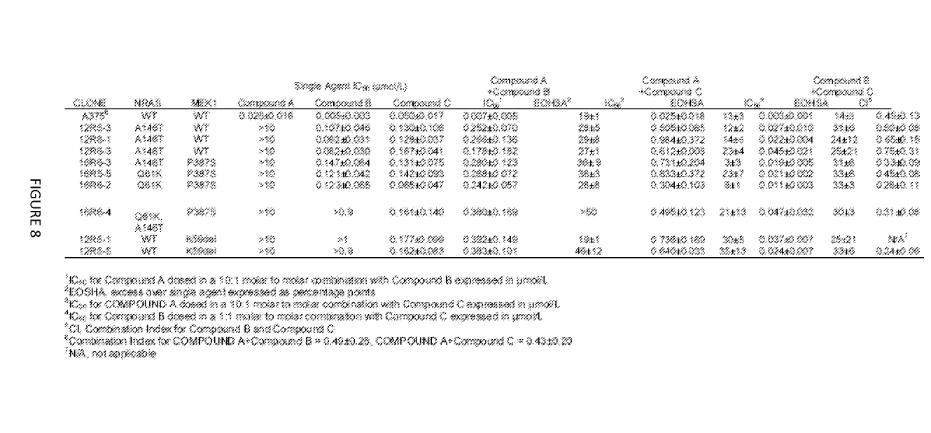

reduced sensitivity to the MEK inhibitor Compound B compared to parental A375 cells (FIG. 8).

### YUSIT1 Clones

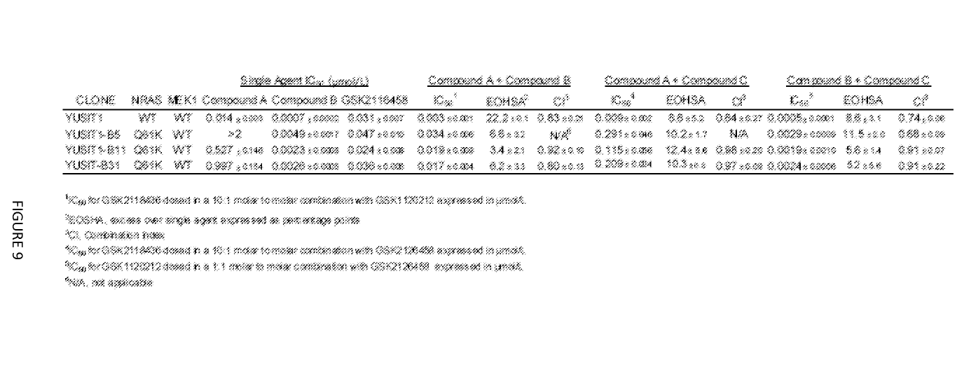

Multiple clones derived from YUSIT1 cells, which have a BRAF V600K mutation, were selected based on their ability to proliferate in the presence of 0.1 umol/L of Compound A. The YUSIT1 clones showed ≧38-fold reduced sensitivity to Compound A (IC₅₀>0.5 μmol/L) and 3-7 fold reduced sensitivity to Compound B (IC₅₀s, 0.002-0.005 μmol/L) compared to the parental YUSIT1 cells (FIG. 9). Resistant clones derived from YUSIT1, as with those derived from A375, acquired NRASQ61K in three of the three clones tested. These clones retained BRAFV600K and MEK2E27K.

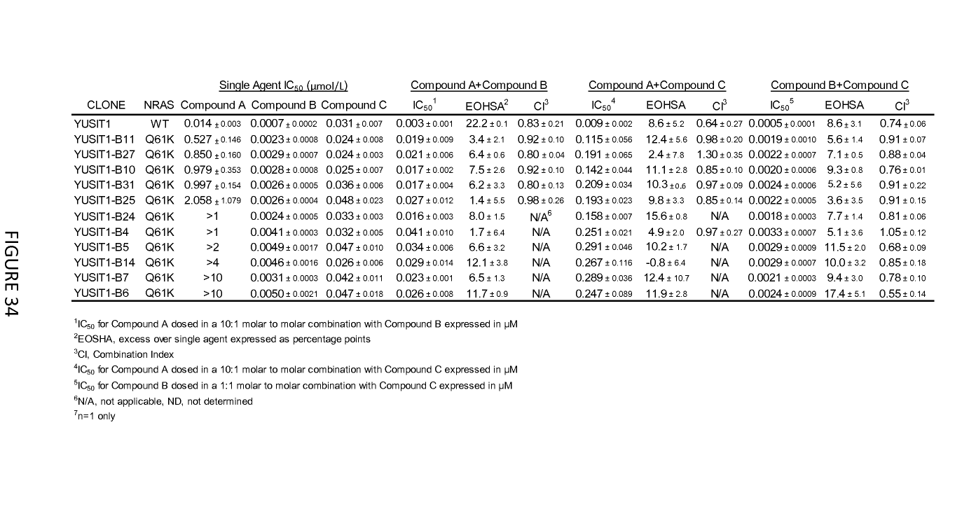

As shown in FIG. 34, the YUSIT1 clones that acquired resistance to Compound A, displayed reduced sensitivity to Compound B and similar sensitivity to Compound C. In the YUSIT1 clones, the combination of Compound A and Compound B was slightly synergetic to nearly additive (combination index, CI, 0.8-0.92) with a modest increase in potency (FIG. 9). The combination of Compound A and Compound C was synergistic to nearly additive in the YUSIT1 clones. Similarly, the combination of Compound B and Compound C was synergistic to nearly additive in the YUSIT1 resistant clones. Both combinations modestly increased the potency of the most active single agent.

Elevated MAP Kinase Signaling in Acquired Resistance Clones Treated with Compound A

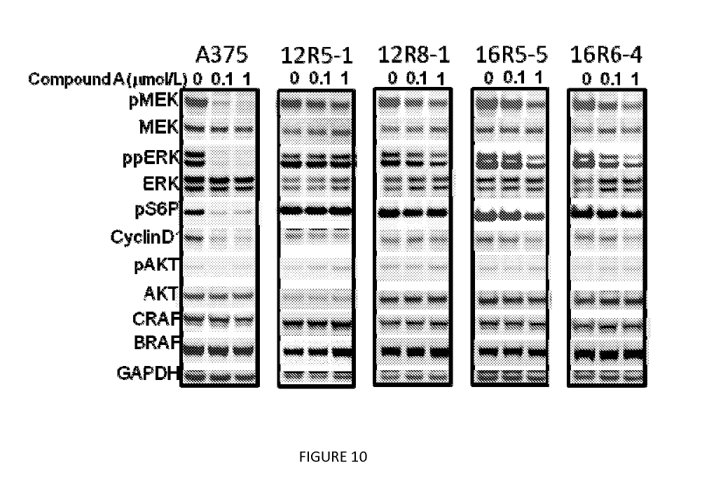

Four representative A375 clones were treated with Compound A and analyzed for signaling effects by western blot compared to parental clone (FIG. 10). In the absence of Compound A, both A375 and the resistant clones showed high levels of phosphorylation of MEK, ERK, and S6 ribosomal protein (S6P), and low levels of phosphorylated AKT at T308. ARAF, BRAF and CRAF protein levels were similar between the resistant clones and the parental cells (FIG. 7., data not shown). In A375 cells, Compound A potently inhibited phosphorylation of MEK, ERK, and S6P more than 75% and decreased cyclin D1 protein levels more than 65% (FIG. 10). These effects were attenuated in the resistant clones following treatment with Compound A. For example, MEK phosphorylation was reduced 30 to 70% while ERK and S6P phosphorylation were reduced by less than 50% and 25%, respectively. Unlike the parental line, cyclin D1 protein levels remained high in the clones following Compound A treatment.

### NRAS Mutations Mediate Resistance to Compound A

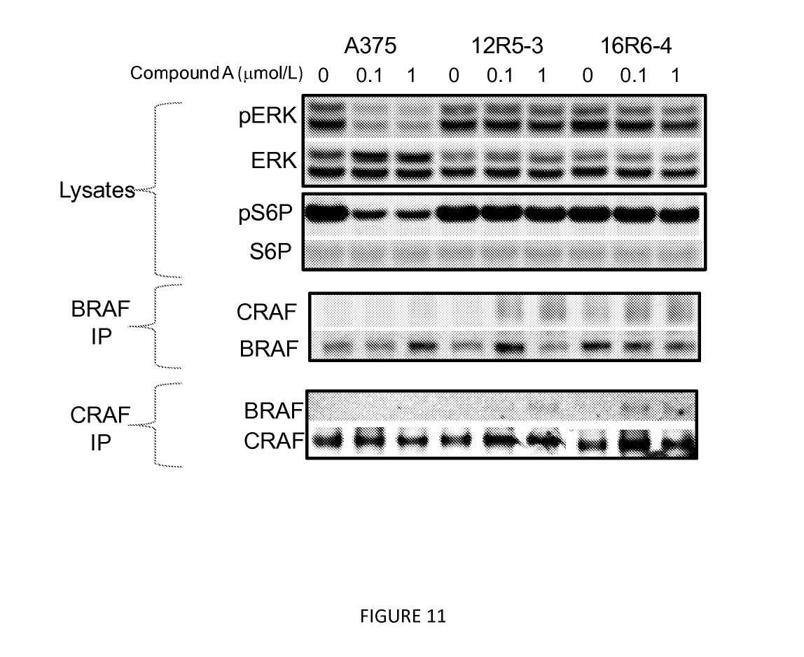

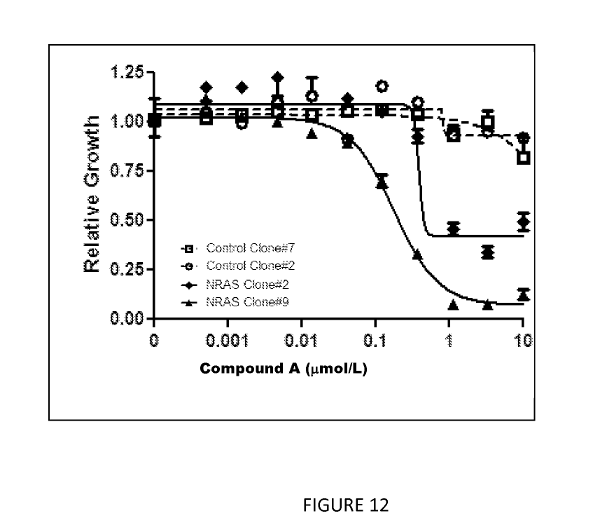

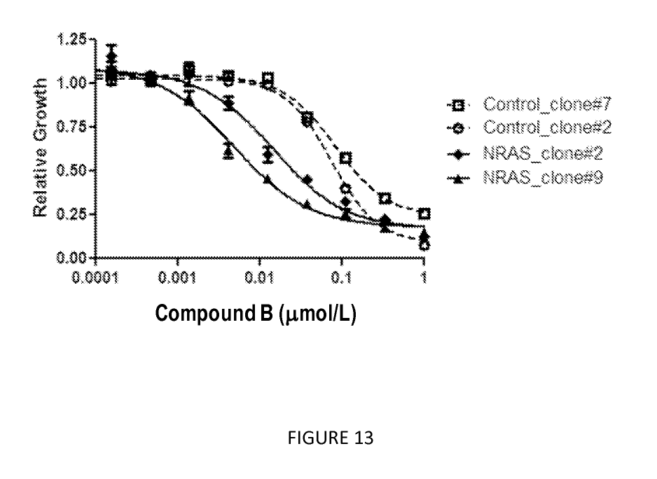

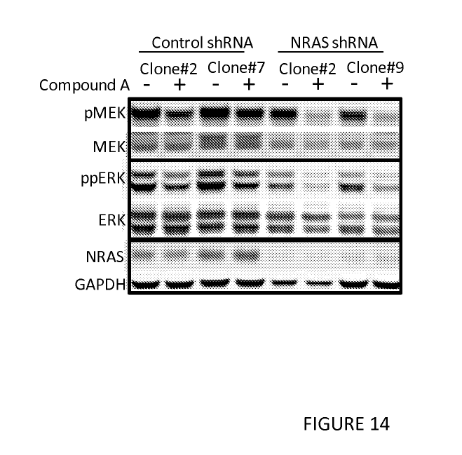

Next, the effect of the observed NRAS mutations on Compound A and Compound B sensitivity was evaluated in three representative clones with NRASA146T (12R5-3 and 12R8-1) or NRASQ61K and NRASA146T (16R6-4). Activated RAS levels were increased 2.5 to 7-fold in these clones over the parental (FIG. 3, data not shown). Compound A increased the co-precipitation of CRAF with BRAFV600E in both 12R5-3 (NRASA146T) and 16R6-4 (NRASQ61K and NRASA146T) (FIG. 11). In 16R6-4, stable reduction of NRAS by shRNA partially restored the sensitivity to Compound A (FIG. 12) and Compound B (FIG. 13) and the ability of Compound A to decrease phosphorylation of MEK and ERK (FIG. 14). Partial restoration of sensitivity to Compound A and Compound B was also observed in 12R5-3 and 12R8-1 following stable knockdown of NRAS (data not shown).

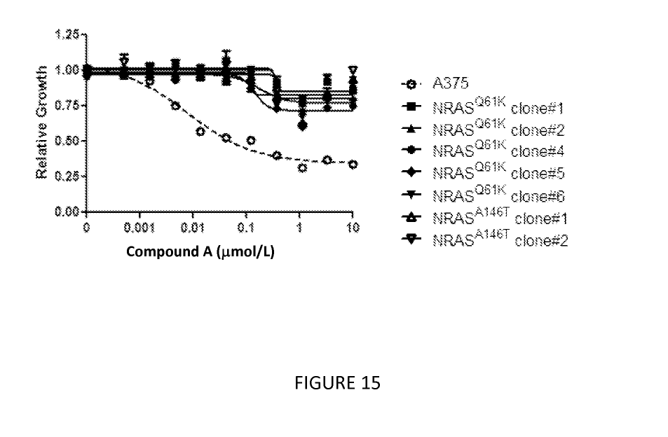

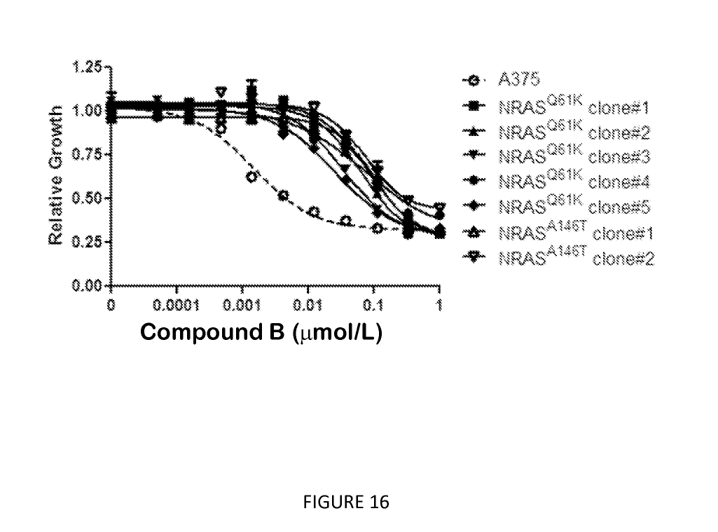

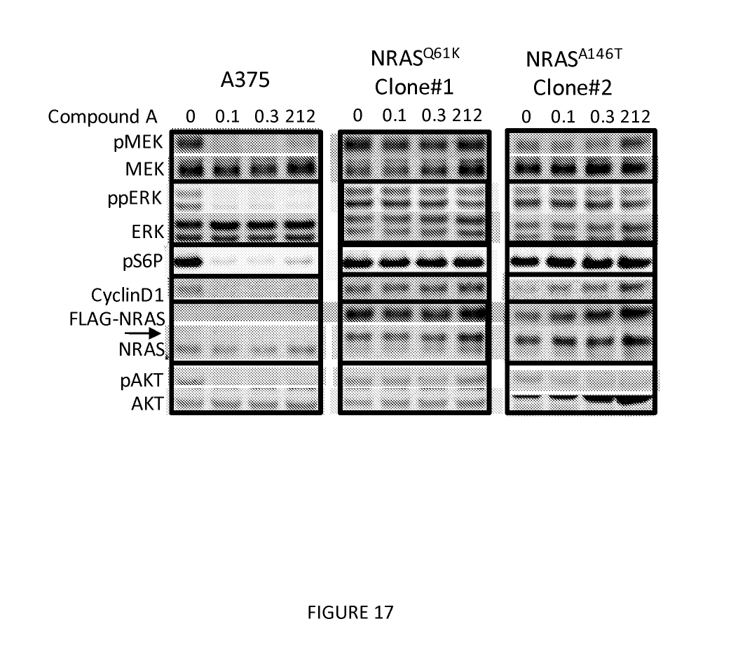

To further confirm that NRASQ61K or NRASA146T mutations promote resistance to Compound A, plasmids encoding these mutations and the neomycin resistance gene were transfected into A375 cells. Selection with G418 generated clones that did not express mutant NRAS (data not shown). However, selection with a low dose of Compound A (0.1 μM) resulted in clones expressing mutant NRAS. Mock or wild-type NRAS transfections failed to form colonies when selected with Compound A. The mutant NRAS expressing clones were insensitive to cell growth inhibition by Compound A (FIG. 15,) and Compound B (FIG. 16), while also failing to reduce MEK, ERK, and S6P phosphorylation, and cyclin D1 protein after Compound A treatment (FIG. 17).

### MEK Mutations Confers Resistance to Compound a

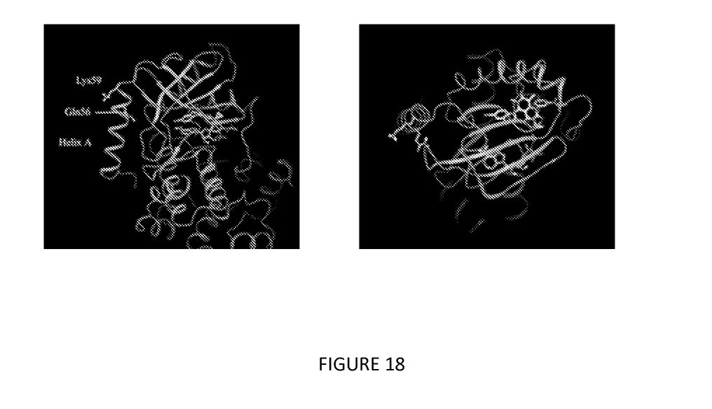

Next, whether the K59del and P387S MEK mutations found in the Compound A resistant clones may activate MEK and promote resistance to Compound A and Compound B was investigated. K59 is located adjacent to the negative regulatory helix preceding the core kinase domain of MEK. Helix A has been hypothesized to stabilize an inactive conformation of MEK1. This conformation is the same, or similar to, the conformation that binds allosteric inhibitors like Compound B shown in FIG. 18. Mutations in this regulatory helix, such as MEK1Q56P, have been shown to activate non-phosphorylated MEK1 and decrease sensitivity to BRAF and MEK inhibitors (Emery C M et al., Proc Natl Acad Sci USA 2009, 106:20411-6) P387 is located in the largely disordered C-terminus of MEK1 adjacent to T386, which is involved in negative regulation of MEK1 (Fischmann T O, et al. Biochemistry, 2009, 48: 2661-74).

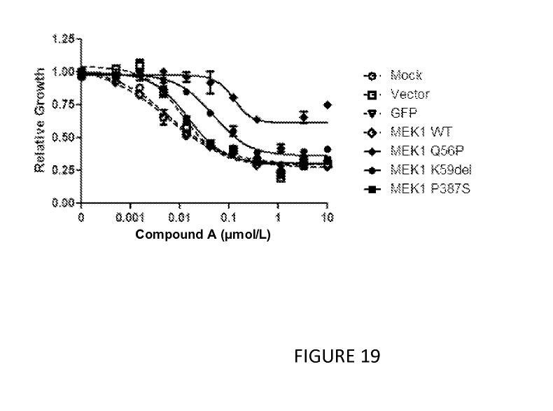

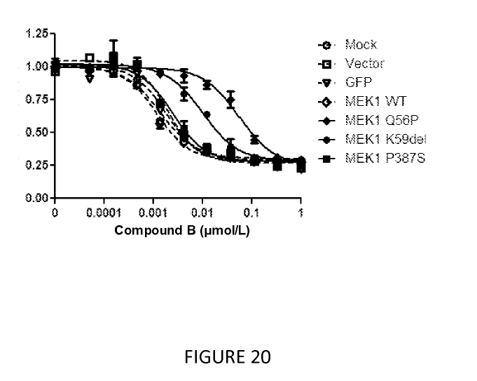

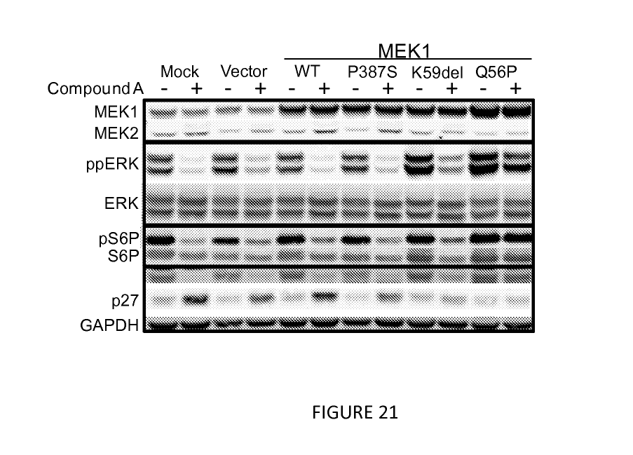

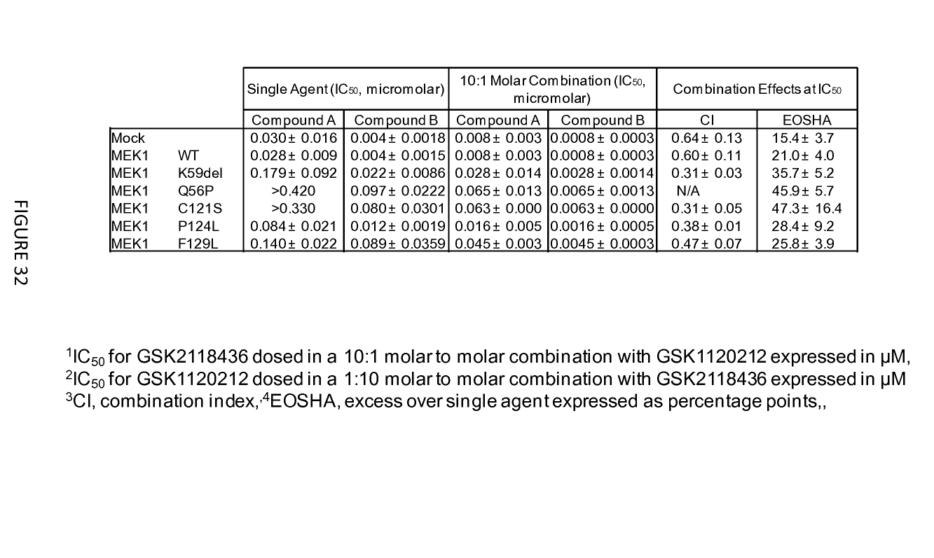

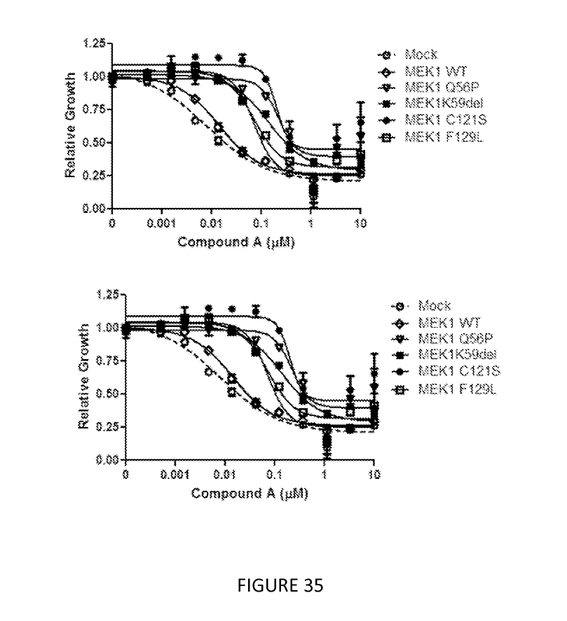

As shown in FIGS. 19, 32, and As shown in FIGS. 20, 35, and 36 expression of MEK1K59del, MEK1Q56P, MEK1C121S, MEK1P124L and MEK1F129L in A375 cells decreased the ability of Compound A to inhibit proliferation (IC50=0.179 μM, ≧0.420 μM, ≧0.330 μM, 0.084 μM and 0.140 μM, respectively) compared to Mock and wild-type MEK1 (IC50s=0.030 μM and 0.028 μM). Expression of MEK1K59del, MEK1Q56P, MEK1C121S, MEK1P124L, and MEK1F129L, reduced sensitivity to Compound B (FIG. 20). Consistent with the proliferation data, only MEK1Q56P and MEK1K59del sustained ERK phosphorylation following treatment with Compound A (FIG. 21). Likewise, p27kip1 induction was absent in cells expressing MEK1Q56P and MEK1K59del. Lack of inhibition of S6P phosphorylation was most profound in MEK1Q56P, but still appreciable in MEK1K59del. Both MEK1Q56P and MEK1K59del displayed increased basal phosphorylation of ERK.

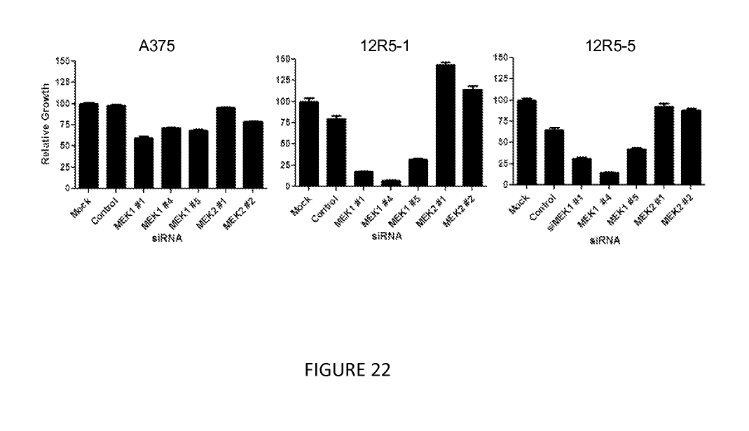

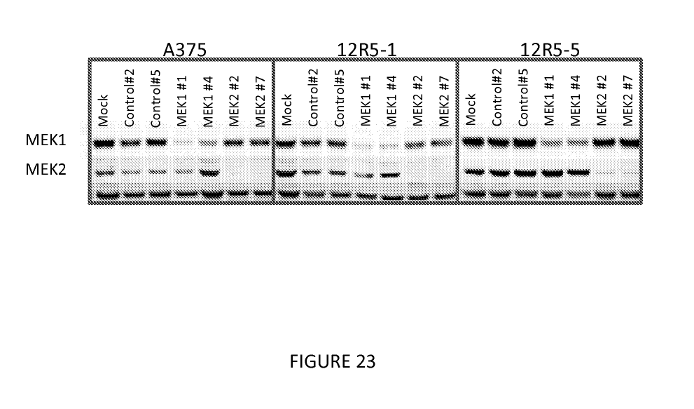

To confirm MEK mutations contribute to Compound A resistance in our A375 clones, small interfering RNA (siRNA) were used to knockdown MEK1 in 12R5-1 and 12R5-5. In the absence of compound, these clones were very sensitive to MEK1 downregulation, as cell proliferation was reduced >75% in 12R5-1 and >60% in 12R5-5, indicative of MEK1 dependence (FIG. 22, FIG. 23). MEK2 knockdown had no effect on proliferation.

### Compound A Acquired Resistant Melanoma Cells are Sensitive to the Combination of Compound A and Compound B

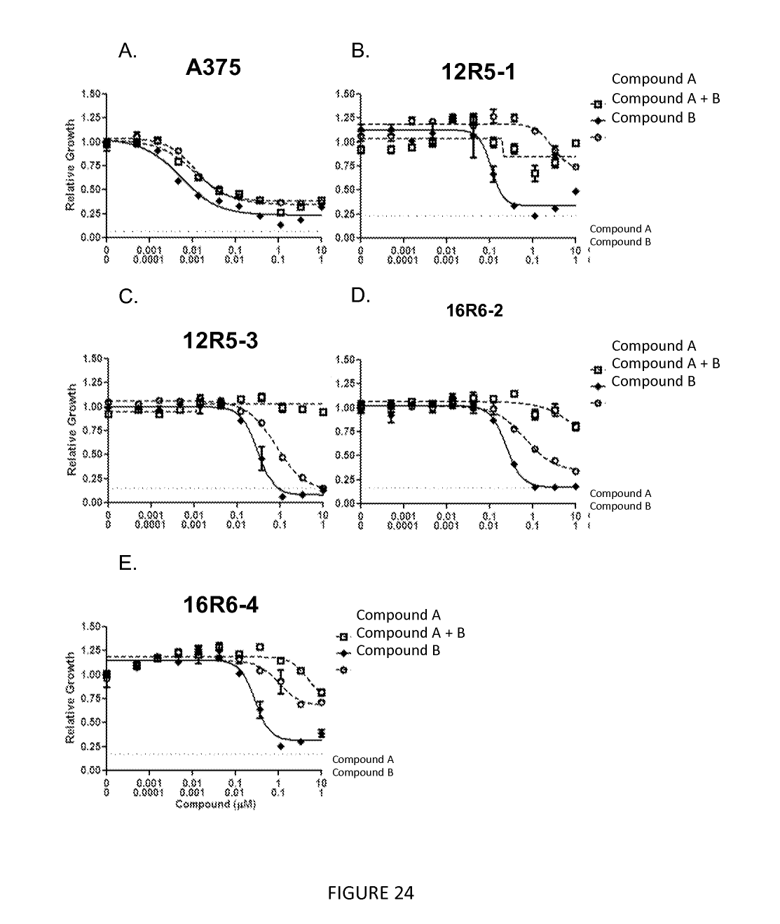

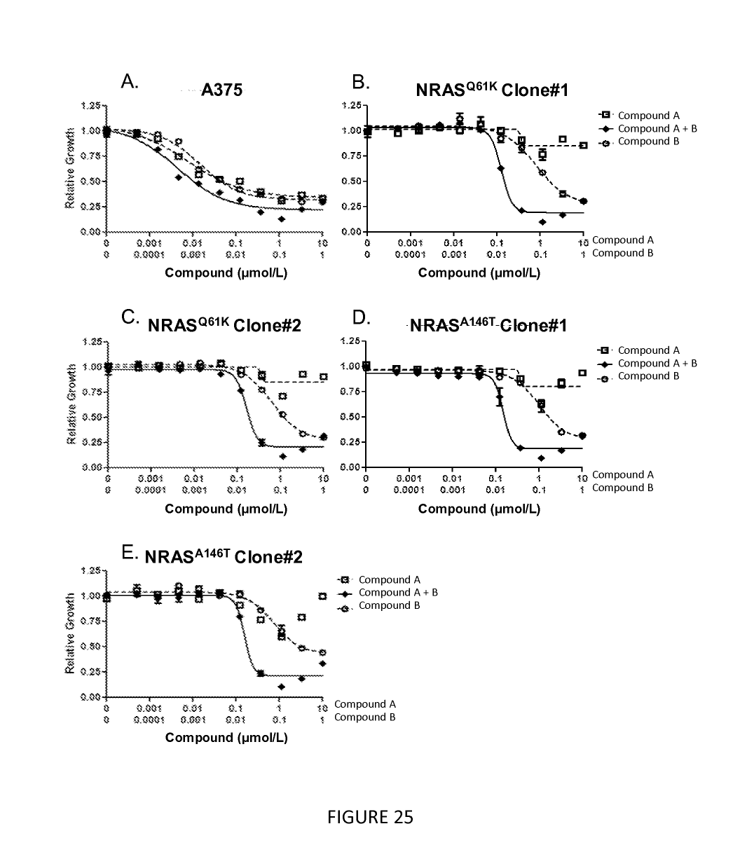

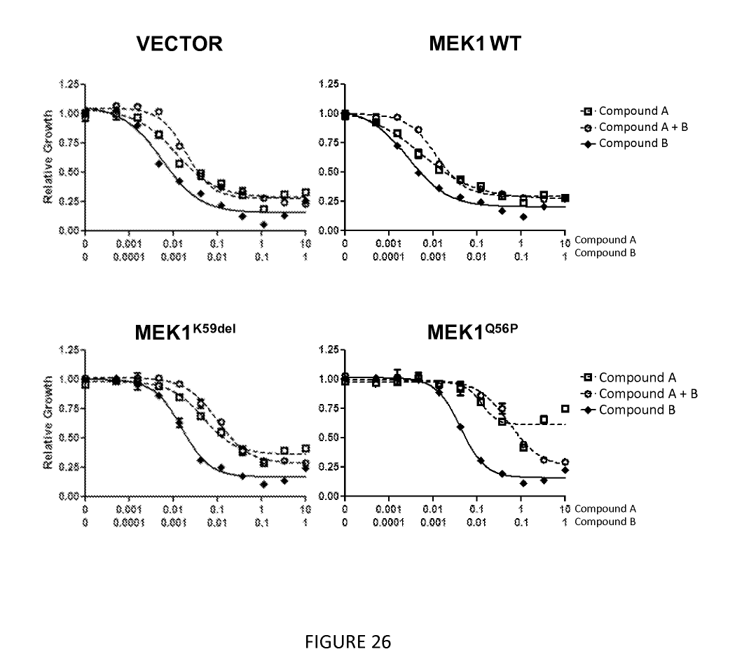

Because resistant clones showed sustained ERK phosphorylation following Compound A, whether combined inhibition of BRAF and MEK could overcome this resistance was tested. Combination of Compound A and Compound B at a constant molar ratio of 10:1 enhanced growth inhibition in all acquired resistant clones irrespective of NRAS or MEK1 mutation (FIG. 8). Although a combination index could not be calculated due to the lack of Compound A single agent activity, excess over highest single agent (EOSHA) showed a benefit of a 19-50% increase in cell growth inhibition at the IC50, when compared to Compound B alone (FIG. 8 and FIG. 24). A similar benefit with the combination of Compound A and Compound B was observed in the A375 cells that stably expressed NRASQ61K and NRASA146T (FIG. 25) or were transduced with MEK1K59del and MEK1Q56P or were transduced with MEK1K59del, MEK1Q56P, MEK1C121S, MEK1P124Land MEK1F129L (FIG. 26, FIG. 32).

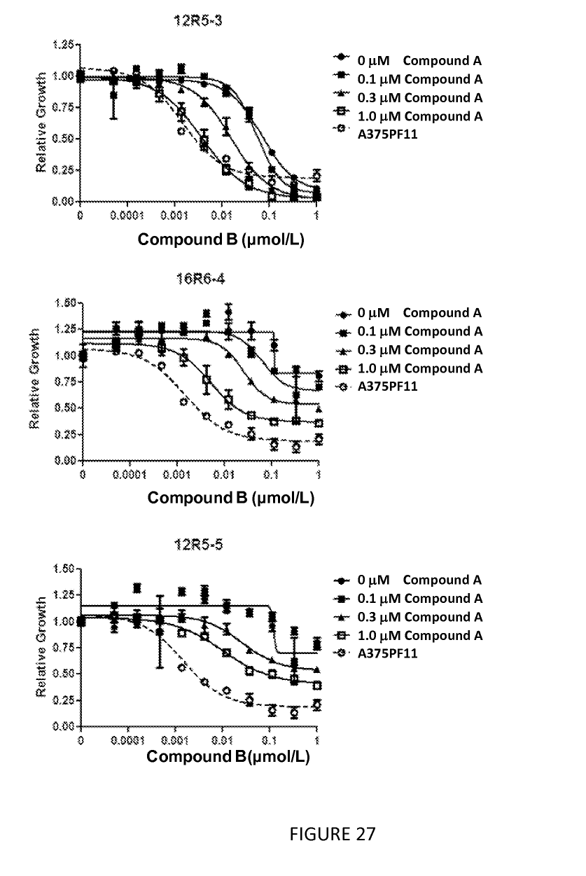

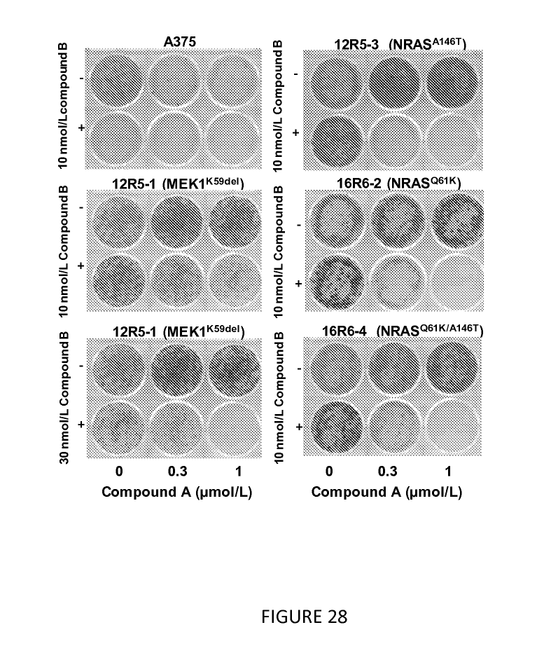

One micromolar Compound A nearly restored the sensitivity to Compound B in all clones with acquired NRAS mutations (FIG. 27). However, similar dosing restored only partial sensitivity to Compound B in the clones 16R6-4 (NRASQ61K and NRASA146T) and 12R5-5 (MEK1K59del). In an extended proliferation assay with acquired resistant clones, combination of 1 μM Compound A with 0.01 μM Compound B inhibited >90% of cell growth in the NRAS mutant clones (FIG. 28). Growth inhibition in NRAS mutant clones was more pronounced than in the MEK1K59del clone, which required higher concentration of Compound B to observe a similar effect.

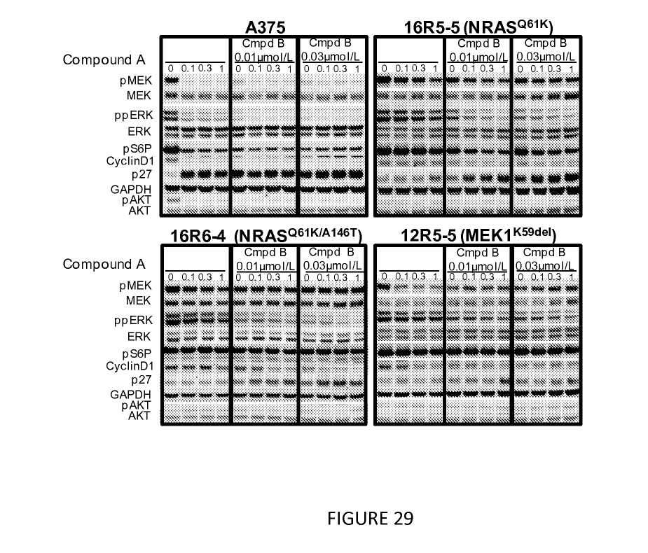

To characterize the combination of Compound A and Compound B further, the effect of this combination on the RAF-MEK-ERK pathway was examined. As shown in FIG. 29, single agent Compound A or Compound B effectively reduced ERK and S6P phosphorylation in A375. These inhibitors were sufficient to reduce cyclin D1 and increase p27kip1. In combination, ERK and S6P phosphorylation were further reduced, whereas, cyclin D1 or p27kip1protein levels showed little enhancement. In a representative subset of the acquired resistant clones, Compound A alone had little effect on ERK or S6P phosphorylation or cyclin D1 and p27kip1 protein levels. Compound B alone reduced phosphorylation of ERK. In combination, ERK phosphorylation levels in the NRAS mutant clones were reduced to that observed in A375 treated with Compound A. The effect on ERK phosphorylation with this combination treatment was attenuated in the MEK1K59del clones. In addition, combination dosing of the clones decreased cyclin D1, and increased p27kip1, although not to the same extent as A375 treated with Compound A. Varying degrees of S6P phosphorylation reduction were observed in the clones treated with combination. AKT phosphorylation was slightly reduced or unchanged in both A375 and the resistant clones.

As the combination of Compound A and Compound B reduced signaling and proliferation of the clones that acquired resistance to Compound A, the global gene regulation effect of this combination in the 16R6-4 clone (NRASQ61K and NRASA146T, MEK1P387S) was evaluated. Consistent with observed resistance in 16R6-4 cells, Compound A treatment altered few genes, which were related to metabolism (PHGDH, PCK2, ASNS) and small molecule transport (SLC1A4, CHAC1). The addition of Compound B to Compound A altered gene expression in a manner that substantially overlapped with changes observed in A375 cells treated with this combination (FIG. 9, data not shown). As determined using microarray analysis described above, the combination in the resistant clone similarly down-regulated genes involved in cell proliferation and survival (e.g., CCND1, CDC25A, PCNA, MYC, MCL1) as Compound A or Compound B treatment of the parental cells (data not shown). Transcripts associated with apoptosis (e.g., BIK, CASP1, CARD6) were up-regulated in the resistant clone treated with the combination but not to the same extent as in the parental cells treated with either single agent. The combination in both 16R6-4 and A375 decreased transcripts dependent on MEK-ERK signaling (e.g., DUSP4, DUSP5, DUSP7, ETV1, ETV4, FOXC2, SPRY2) and up-regulated transcriptions related to alternative pathways (e.g., RAB26, RHOB, SNAI2). The data demonstrate that the combination of Compound A and Compound B nearly restored the gene expression associated with RAF or MEK inhibition in the parental, leading to profound cell growth inhibition. Inhibition of both MAPK and the PI3K/mTOR pathways enhance cell growth inhibition in the clones with acquired resistance to Compound A.

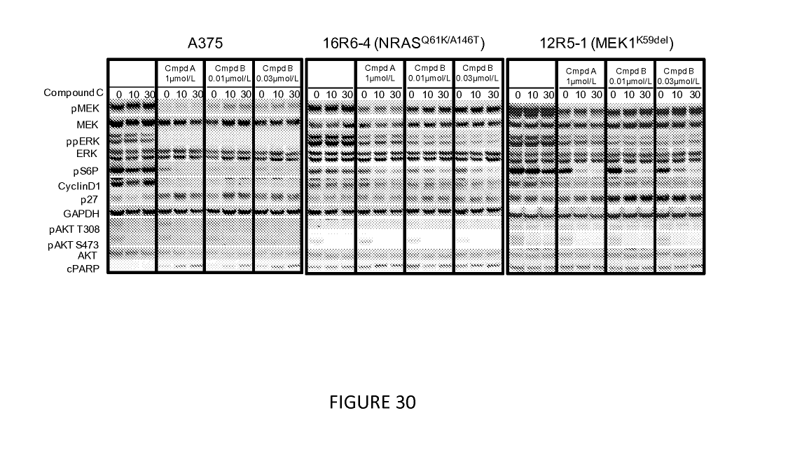

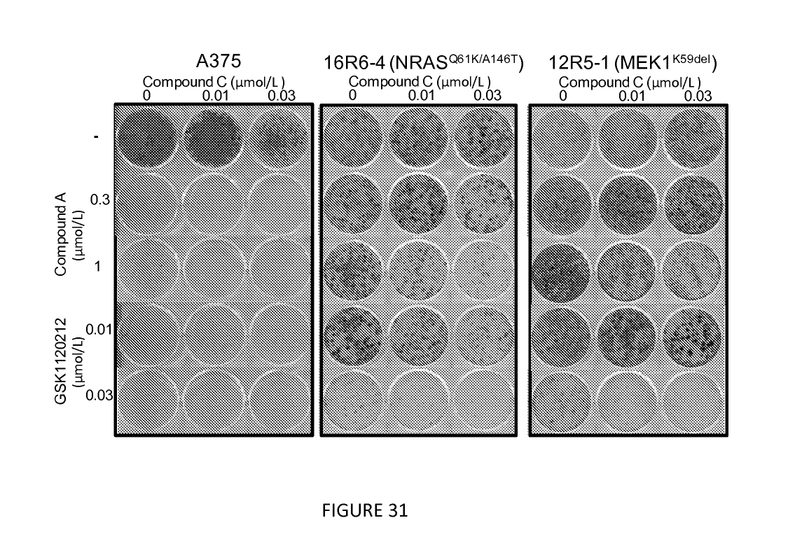

While the combination of Compound A and Compound B profoundly inhibited proliferation of the resistant clones, residual S6P phosphorylation remained in the clones. Since S6P phosphorylation can be induced by activation of the PI3K/mTOR pathway, the combination of Compound A or Compound B with Compound C. All clones displayed modest sensitivity to Compound C was evaluated (FIG. 8). Compound C treatment of representative resistant clones decreased AKT phosphorylation and slightly decreased S6P phosphorylation (FIG. 30). The addition of Compound A or Compound B to Compound C further reduced S6P phosphorylation and cyclin D1 protein. Phosphorylation of MEK and ERK was similar to treatment with COMPOUND A or Compound B alone. In some clones, such as 16R6-4, cleaved PARP, an indicator of apoptosis, was elevated with the combination of Compound C with COMPOUND A or Compound B. Other clones, such as 12R5-1, displayed high basal levels of cleaved PARP. The addition of Compound A to Compound C enhanced cell growth inhibition (EOSHA >10 ppts) in 5/7 clones with NRAS mutations and the two MEK1K59del clones (FIG. 8). The combination of Compound B and Compound C was synergistic (CI<0.8) in 8/9 clones and enhanced cell growth inhibition (EOSHA >20 ppts) in all nine clones regardless of NRAS or MEK1 mutation status (FIG. 8). Long term proliferation assays confirmed the enhancement of growth inhibition by the combination of COMPOUND A or Compound B with Compound C (FIG. 31). In general the resistant clones were more sensitive to cell growth inhibition with the combination of Compound C and Compound B at the concentrations used; however, appreciable activity was observed with 1 μmol/L Compound A and 0.03 μmol/L Compound C. The anti-proliferative effect of Compound A in combination with Compound C was not as potent as observed with the Compound A and Compound B combination, as well as the Compound B and Compound C combination. Similar benefits were observed for these combinations in the YUSIT1 clones with NRAS mutation (FIG. 9).

1. A method of treating melanoma exhibiting a V600 mutation in a human in need thereof, comprising
determining if the melanoma exhibits at least one of an NRAS Q61 mutation, NRAS A146 mutation, MEK1 K59 deletion, or MEK1 P387 mutation, and
if said melanoma exhibits at least one of an NRAS Q61 mutation, NRAS A146 mutation, MEK1 K59 deletion, or MEK1 P387 mutation, then administering to said human an effective amount of at least one of:
a combination of a PI3K inhibitor and MEK inhibitor, or
a combination of a BRAF inhibitor and MEK inhibitor.

2. A method of treating melanoma exhibiting a V600 mutation in a human in need thereof, comprising
determining if the melanoma exhibits at least one of an NRAS Q61 mutation, NRAS A146 mutation, MEK1 K59 deletion, or MEK1 P387 mutation, and
if said melanoma exhibits at least one of an NRAS Q61 mutation, NRAS A146 mutation, MEK1 K59 deletion, or MEK1 P387 mutation, then administering to said human an effective amount of at least one of:
a combination of a PI3K inhibitor and MEK inhibitor, or
a combination of a BRAF inhibitor and MEK inhibitor, or
a combination of a BRAF inhibitor and a PI3K inhibitor.

3. A method of treating melanoma exhibiting a V600 mutation in a human in need thereof, comprising
determining if the melanoma exhibits at least one of an NRAS Q61 mutation, NRAS A146 mutation, MEK1 K59 deletion, or MEK1 P387 mutation, and
if said melanoma exhibits at least one of an NRAS Q61 mutation, NRAS A146 mutation, MEK1 K59 deletion, or MEK1 P387 mutation, then administering to said human an effective amount of at least one of:
a combination of a PI3K inhibitor and MEK inhibitor, or
a combination of a BRAF inhibitor and MEK inhibitor, or
a combination of a BRAF inhibitor and a PI3K inhibitor, or
a combination of a BRAF inhibitor and MEK inhibitor and PI3K inhibitor.

4. A method of treating melanoma exhibiting a V600 mutation in a human in need thereof, comprising
determining if the melanoma exhibits at least one of an NRAS Q61 mutation, NRAS A146 mutation, MEK1 K59 deletion, MEK1 P387 mutation, MEK1Q56P mutation, MEK1 C121 mutation, MEK1 P124 mutation or MEK1 F129 mutation; and
if said melanoma exhibits at least one of an NRAS Q61 mutation, NRAS A146 mutation, MEK1 K59 deletion, MEK1 P387 mutation, MEK1 Q56 mutation, MEK1 C121 mutation, MEK1 P124 mutation or MEK1 F129 mutation, then administering to said human an effective amount of at least one of:
a combination of a PI3K inhibitor and MEK inhibitor, or
a combination of a BRAF inhibitor and MEK inhibitor, or
a combination of a BRAF inhibitor and PI3K inhibitor, or
a combination of a BRAF inhibitor and MEK inhibitor and PI3K inhibitor.

5. The method of any of the preceding claims, wherein the melanoma exhibits a V600 mutation selected from V600E and V600K.

6. The method of any of the preceding claims, wherein the melanoma exhibits at least one of an NRAS Q61R, NRAS Q61K mutation, NRAS Q61H mutation, NRAS Q61L mutation, NRAS Q61N mutation, NRAS Q61E mutation, NRAS Q61P mutation, NRAS A146T mutation, NRAS A146P mutation, NRAS A146V mutation, MEK1 K59 deletion, MEK1 P387L mutation, MEK1 Q56P mutation, MEK1 C121S mutation, MEK1 P124L mutation, or a MEK1 F129L mutation.

6. The method of claims 1-5, wherein the BRAF inhibitor is a compound having the formula: 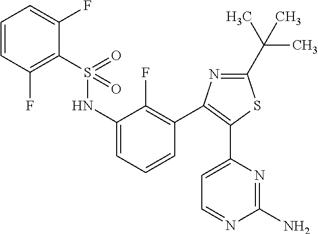 [SMILES: CC(C)(C)c1nc(-c2cccc(NS(=O)(=O)c3c(F)cccc3F)c2F)c(-c2ccnc(N)n2)s1]
, or a pharmaceutically acceptable salt or solvate thereof.

4. The method of claims 1-5, wherein the BRAF inhibitor is a compound (PLX4032) having the formula: 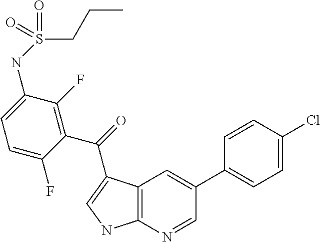 [SMILES: CCCS(=O)(=O)Nc1ccc(F)c(C(=O)c2c[nH]c3ncc(-c4ccc(Cl)cc4)cc23)c1F]
, or a pharmaceutically acceptable salt or solvate thereof.

5. The method of claims 1-4 wherein the MEK inhibitor is a compound having the formula: 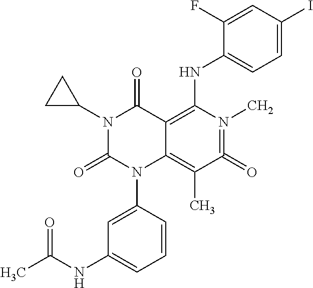
, or a pharmaceutically acceptable salt or solvate thereof.

6. The method of claims 1-5 wherein the PI3K inhibitor is a compound having the formula: 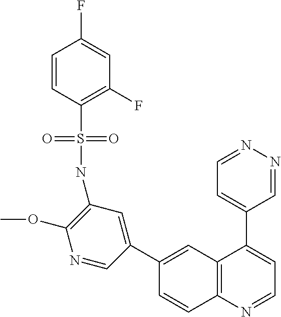 [SMILES: COc1ncc(-c2ccc3nccc(-c4ccnnc4)c3c2)cc1NS(=O)(=O)c1ccc(F)cc1F]
, or a pharmaceutically acceptable salt or solvate thereof.
